(19) United States
(12) Patent Application Publication
(10) Pub. No.: US 2022/0275376 A1
(43) Pub. Date: Sep. 1, 2022

(54) TARGETING SLC38A2 IN PANCREATIC CANCER

(71) Applicant: New York University, New York, NY (US)

(72) Inventors: Alec C. KIMMELMAN, Larchmont, NY (US); Seth J. PARKER, New York, NY (US)

(21) Appl. No.: 17/637,427

(22) PCT Filed: Aug. 20, 2020
(86) PCT No.: PCT/US2020/047218

§ 371 Date: Feb. 22, 2022

Related U.S. Application Data
(60) Provisional application No. 62/889,493, filed on Aug. 20, 2019.
(60) Provisional application No. 62/889,494, filed on Aug. 20, 2019.

(51) Int. Cl. C12N 15/113; A61K 31/15; A61K 31/138; A61K 31/4525; A61K 31/135; A61K 31/55; A61P 35/00; G01N 33/574

(52) U.S. Cl. CPC C12N 15/1138; A61K 31/15; A61K 31/138; A61K 31/4525; A61K 31/135; A61K 31/55; A61P 35/00; G01N 33/57438; C12N 2310/14; C12N 2310/531

(57) ABSTRACT

The present disclosure provides compositions and methods for interfering with uptake of neutral amino acids (e.g., alanine) in pancreatic cells. Alanine uptake can be inhibited by inhibiting the function and/or expression of SLC38A2 and/or SLC1A4.

### CROSS REFERENCE TO RELATED APPLICATIONS

This application claims priority to U.S. Provisional Application Nos. 62/889,493 and 62/889,494, both filed on Aug. 20, 2019, the disclosures of which are incorporated herein by reference.

### STATEMENT REGARDING FEDERALLY SPONSORED RESEARCH AND DEVELOPMENT

This invention was made with government support under contract nos. R01CA157490, R01CA188048, P01CA117969, R35CA232124, and R01GM095567 awarded by the National Institutes of Health. The government has certain rights in the invention.

### BACKGROUND OF THE DISCLOSURE

Pancreatic ductal adenocarcinoma (PDAC) is one of the deadliest forms of cancer with a 5-year survival rate of 8.2%. Currently, the most successful treatment for pancreatic cancer is surgical resection of local disease with a 31.5% 5-year survival according to the National Cancer Institute (NCI/SEER). Unfortunately, only ˜20% of patients present with disease amendable for resection; thus, more effective therapies are needed for improving the outcome for patients with pancreatic cancer.

### SUMMARY OF THE DISCLOSURE

SLC38A2, one of the two neutral amino acid transporters, is highly expressed in pancreatic cancer cells relative to normal tissues and non-transformed cells within PDAC tumors. Genetically targeting SLC38A2 using RNAi or CRISPR/Cas9 in pancreatic cancer cells reveals that expression of SLC38A2 is required for alanine uptake, which is important for supporting PDAC metabolism. Pancreatic cancer cells lacking SLC38A2 fail to rewire their metabolism to compensate for loss of this transporter. Demonstrated herein is that SLC38A2 loss leads to an amino acid homeostatic crisis, which negatively impacts cell proliferation and tumor initiation and growth.

The present disclosure provides compositions and methods for interfering with uptake of neutral amino acids (e.g., alanine) in pancreatic cells. Alanine uptake can be inhibited by inhibiting the function and/or expression of SLC38A2 and/or SLC1A4.

In an aspect, the present disclosure provides compositions comprising inhibitors (e.g., compounds, antibodies, and the like) of alanine uptake. The compounds or antibodies in the compositions may inhibit the expression or function of SLC38A2 and/or SLC1A4 in a pancreatic cell (e.g., a pancreatic cancer cell, such as, for example, a pancreatic ductal adenocarcinoma cell). Non-limiting examples of inhibitors include small molecules (e.g., antidepressants), peptides and/or proteins (e.g., antibodies (an antigen binding fragment thereof or modification thereof)), or RNA molecules (e.g., an interfering RNA (such as, for example, shRNA or siRNA) or dsRNA). The compositions may comprise one or more pharmaceutically acceptable carriers.

In an aspect, the present disclosure provides methods of treating pancreatic cancer (e.g., pancreatic ductal adenocarcinoma). Various examples comprise using one or more inhibitors or compositions thereof. The method may comprise inhibiting SLC38A2 and/or SLC1A4 in a pancreatic cell (e.g., pancreatic cancer cell, such as, for example, a pancreatic ductal adenocarcinoma cell). Inhibiting SLC38A2 and/or SLC1A4 can inhibit alanine uptake in a pancreatic cell (e.g., pancreatic cancer cell, such as, for example, a pancreatic ductal adenocarcinoma cell). Various other examples comprise genetic modification of a pancreatic cancer cell (e.g., pancreatic ductal adenocarcinoma cell). In an example, a method of the present disclosure for treating pancreatic cancer comprises administering to a subject in need of treatment a composition of the present disclosure.

In another aspect, the disclosure includes disrupting the target gene such that SLC38A2 and/or SLC1A4 mRNA and protein are not expressed. In one embodiment, the SLC38A2 and/or SLC1A4 gene can be disrupted by targeted mutagenesis. In embodiments, targeted mutagenesis can be achieved by, for example, targeting a CRISPR (clustered regularly interspaced short palindromic repeats) site in the target gene. So-called CRISPR systems designed for targeting specific genomic sequences are known in the art and can be adapted to disrupt the target gene for making modified cells encompassed by this disclosure. In general, the CRISPR system includes one or more expression vectors encoding at least a targeting RNA and a polynucleotide sequence encoding a CRISPR-associated nuclease, such as Cas9, but other Cas nucleases can alternatively be used. CRISPR systems for targeted disruption of mammalian chromosomal sequences are commercially available.

In an aspect, the present disclosure provides methods for identifying whether a tumor (e.g., a pancreatic tumor) is cancerous or non-cancerous.

In an aspect, the present disclosure provides methods of identifying inhibitors for SLC38A2 and/or SLC1A4 and inhibiting alanine uptake. Methods may be experimental and/or in silico.

### BRIEF DESCRIPTION OF THE FIGURES

For a fuller understanding of the nature and objects of the disclosure, reference should be made to the following detailed description taken in conjunction with the accompanying figures.

FIG. 1 shows heterogeneous alanine fate and differential neutral amino acid transporter expression in PDAC and pancreatic stellate cells. (A) Alanine uptake and secretion flux in a panel of human and mouse PDAC and stellate cell lines cultured in DMEM or DMEM supplemented with 1 mM L-alanine. Extracellular accumulation (+, secretion) or depletion (−, uptake) was measured in conditioned media over 24-72 hours and normalized to the viable cell density over the time course. Error bars depict s.d. of three independent experiments. (B) Alanine exchange flux as compared to net secretion flux and substrate-inhibited flux in human and mouse pancreatic stellate cell lines. ¹³C₃-alanine dilution by unlabeled (M+0) alanine was measured over time, and the molar exchange was quantified and normalized by viable cell density over 24 hours to quantify the exchange flux. Error bars depict s.d. of three independent experiments. (C) Atom transition map summarizing ¹³C₃-alanine and ¹⁵N-alanine contributions to PDAC intracellular metabolism in a diverse panel of human and mouse PDAC cell lines. Large, grey circles depict ¹³C atom transitions through central carbon metabolism; small, black circles depict ¹⁵N atom transitions through the transaminase network. Unlabeled (¹²C, ¹⁴N) atoms depicted as white circles. (D) Volcano plot of differentially expressed transporters (‘SLC’ proteins) between hPSC #1 and PANC1 cell lines. Transporters were considered differentially expressed if log₂ fold change (FC) ≥2 and p-value ≤0.05. Transporters involved in amino acid transport are highlighted with dark squares and labeled. (E) Relative protein expression across panel of non-malignant pancreatic and PDAC cell lines quantified by summing reporter ion counts of peptide-spectral matches for SLC1A4 and SLC38A2. Error bars depict s.d. of two tandem mass tag-labeled biological replicates for each cell line. (F) Immunoblot of deglycosylated SLC38A2, SLC1A4, N/K-ATPase, and actin in whole cell lysates (20 μg) collected from hPSC #1 and PANC1. Representative immunoblot depicted of five independent immunoblots.

FIG. 2 shows SLC38A2 facilitates alanine uptake in PDAC and is critical for alanine-stimulated growth in nutrient-limiting conditions. (A) Alanine secretion (upper panel) and uptake (lower panel) flux in HY19636, MiaPaCa2, and PANC1 cells cultured in either basal DMEM or DMEM supplemented with 1 mM L-alanine for 24 hours. SLC38A2 expression was suppressed by CRISPR/Cas9 using two sgRNAs targeting SLC38A2 (sgSLC38A2 #1, #3) or a control sgRNA targeting Tomato (sgTom). All experiments were conducted using pools of cells within 1-2 passages after selection. Error bars depict s.d. of three independent experiments. (B) SLC38A2-deficient and control HY19636 cells were supplemented with 1 mM L-alanine tert-butyl ester for 24 hours. Esterified alanine internalization, de-esterification, and secretion was measured by quantifying alanine release into conditioned media and normalizing to viable cell density over the 24 hour time course. Error bars depict s.d. of three independent replicates. (C) Diagram depicting role of SLC38A2 in mediating sodium-dependent concentrative uptake of alanine in PDAC. Cells lacking SLC38A2, rely on passive diffusion through other transporter(s) that cannot sustain alanine influx or maintain intracellular concentrations. Cell-permeable esterified alanine is rapidly released through passive diffusers in cells lacking SLC38A2. (D) Control (sgTom) or SLC38A2-deficient HY19636, MiaPaCa2, and PANC1 cells cultured in low amino acid DMEM (10%) supplemented with or without 1 mM L-alanine for 48 hours. Enhanced proliferation with L-alanine supplementation reported as a percent increase relative to growth of cells cultured in basal low amino acid DMEM without L-alanine. Error bars depict s.e.m. of 4-5 independent experiments of 12 technical replicates each.

FIG. 3 shows loss of SLC38A2 suppresses cell proliferation in replete conditions through homeostatic amino acid crisis. (A) Cell proliferation of HY19636, MiaPaCa2, and PANC1 control (sgTom) and SLC38A2-deficient (sgSLC38A2 #1, #3) cells in basal DMEM over 120 hours. Data are plotted as cell proliferation relative to day 0 collected after cell attachment. Error bars depict s.d. of four independent experiments. (B) Increase and decrease in intracellular amino acid levels in SLC38A2-deficient cells (sgSLC38A2 #1) relative to control cells (sgTom). Data are plotted as percent increase or decrease in SLC38A2-deficient cells relative to control cells. Error bars depict s.d. of ≥3 independent experiments. Dotted line indicates no change and positive and negative values indicate an increase and decrease, respectively, in amino acid levels in SLC38A2-deficient cells. (C) Diagram depicting generation of dox-inducible SLC38A2 cells lacking endogenous SLC38A2 locus deleted via CRISPR/Cas9. Dox-inducible SLC38A2-expressing cell lines were cultured in the presence of doxycycline (0.5 μg/mL) or absence of doxycycline to generate SLC38A2-expressing or chronic SLC38A2-null cell lines over three passages. For metabolomics experiments, doxycycline was acutely withdrawn from SLC38A2-expressing cells and metabolites were collected over the course of 32 hours and compared to metabolite levels extracted from cells cultured in doxycycline or chronic SLC38A2-null cells. (D and E) Intracellular alanine (D) and aspartate (E) levels after acute SLC38A2 loss from doxycycline withdrawal compared to levels measured in SLC38A2-expressing cells (+dox) or chronic SLC38A2-deficient cells (−dox chronic) collected over the same time course. Error bars depict s.d. of three biological replicates. (F) Diagram depicting the percent decrease (negative %) or increase (positive %) in intracellular amino acids between chronic SLC38A2-deficient cells and SLC38A2-expressing cells (+dox) at the 24 hour time point. Substrates of SLC38A2 were identified as amino acids that dropped acutely to chronic levels after 12 hours withdrawal of dox. Secondary effects were identified as amino acids that dropped below chronic levels after 12 hours withdrawal of dox before reaching chronic levels at 24 and 32 hours. Error bars depict s.d. of three biological replicates.

FIG. 4 shows SLC38A2 is vital for PDAC tumor initiation and growth. (A) SLC38A2 staining in pancreatic tumors derived from the KPC (LSL-Kras^{G12D}; Trp53^{lox/+}; p48Cre+) mouse model and normal murine pancreas. Representative fields from KPC (4×, left) and normal pancreas (10×, right) are depicted with scale bars. Arrows indicate instances of punctate SLC38A2 in normal pancreas (right). (B) Representative live cell images of HY19636 (left) and mPSC #1 (right) cells transiently transfected with 3 μg of SLC38A2-GFP (green) overnight and stained with MitoTracker (red) to visualize individual cells; scale bars are indicated in figures. (C) Tumor initiation was significantly suppressed by SLC38A2 knockdown (shSLC38A2 #1, #4) compared to control (shGFP) PANC1 cells (2×10⁵) co-injected with hPSC #1 cells (1×10⁶) in a subcutaneous xenograft model. Subcutaneous tumors were monitored bi-weekly by caliper measurement and considered formed if length and width were measured to be ≥1 mm each. (D) Tumor initiation was significantly suppressed by SLC38A2 knockdown (shSLC38A2 #1, #4) compared to control (shGFP) PANC1 cells (5×10⁵) in a orthotopic xenograft model. Orthotopic xenografts were monitored by 3-D ultrasound bi-weekly and considered tumors if volume ≥1 mm³. (E) Knockout of SLC38A2 significantly reduced tumor burden in HY19636 and PANC1 orthotopic syngeneic allograft and xenograft models, respectively. HY19636 (2.5×10⁴) or PANC1 (5×10⁵) control (sgTom) or pooled SLC38A2-deficient (sgSLC38A2 #1, #3) cells were injected orthotopically, and tumors were weighed following resection after 27 days (HY19636) or 63 days (PANC1). Error bars depict s.e.m. of tumors resected from 9-10 mice.

FIG. 5 shows PDAC cell influx alanine and pancreatic stellate cells rapidly exchange alanine. (A) Alanine, serine, glycine, proline, glutamine, and glutamate extracellular fluxes in a panel of human PDAC cell lines cultured in DMEM. Extracellular accumulation (+, secretion) or depletion (−, uptake) was measured in conditioned media over 24-72 hours and normalized to the viable cell density over the time course. Error bars depict s.d. of three independent experiments. (B) Extracellular alanine concentrations in mM in DMEM or DMEM supplemented with 1 mM L-alanine conditioned by human and mouse stellate cell lines (hPSC #1, mPSC #1) over the course of 72 hours. Error bars depict s.d. of three independent experiments. (C) Intracellular alanine labeling in human and mouse stellate cell lines (hPSC #1, mPSC #1) cultured in DMEM supplemented with 1 mM ¹³C₃-alanine over the course of 72 hours. Unlabeled (M+0) alanine increases over time, whereas uniformly ¹³C-labeled (M+3) alanine decreases over time as extracellular alanine is diluted by exchange with synthesized alanine (M+0). Error bars depict s.d. of three independent experiments.

FIG. 6 shows heterogeneous fate of alanine-derived carbon and nitrogen in PDAC. (A) Atom transition map of ¹³C₃, ¹⁵N-labeled alanine. ¹³C-labeled carbon derived from alanine labels downstream TCA intermediates (e.g., pyruvate, citrate) and contributes carbon to de novo lipogenic pathways. ¹⁵N-labeled nitrogen derived from alanine feeds into transaminase pathways. Large, grey circles depict ¹³C atom transitions through central carbon metabolism; small, black circles depict ¹⁵N atom transitions through the transaminase network. Unlabeled (¹²C, ¹⁴N) atoms depicted as white circles. (B) Intracellular alanine labeling from ¹³C₃-alanine (left) or ¹⁵N-alanine (right) in a panel of human PDAC cell lines cultured in DMEM supplemented with 1 mM L-alanine (¹³C₃- or ¹⁵N-labeled) for 24 hours. Error bars depict s.d. of three independent experiments. (C) Intracellular alanine labeling from ¹³C₃-alanine decreases in human PDAC cells over time, failing to reach isotopic steady state. PDAC cells were cultured in DMEM supplemented with 1 mM ¹³C₃-alanine for 24 or 48 hours. Error bars depict s.d. of three independent experiments. (D) Citrate M+2 labeling normalized to alanine labeling (M+3) in human PDAC cell lines cultured in DMEM supplemented with 1 mM ¹³C₃-alanine for 24 hours. Error bars depict s.d. of three independent experiments. (E) Palmitate labeling (M+0, M+2, M+4, etc.) in PDAC cells cultured with ¹³C₃-alanine for 24 hours. Error bars depict s.d. of three independent experiments. (F, G, and H) Labeled transaminase products (M+1) normalized to intracellular alanine labeling from ¹⁵N-alanine in human PDAC cell lines cultured in DMEM supplemented with 1 mM ¹⁵N-alanine for 24 hours and associated atom transition maps for each transaminase pathway. Error bars depict s.d. of three independent experiments. (I) Alanine carbon (left panel) and nitrogen (right panel) contribution to proteinogenic amino acids in human PDAC cells cultured in DMEM supplemented with 1 mM of either ¹³C₃-alanine or ¹⁵N-alanine for 24 hours. Error bars depict s.d. of three independent experiments.

FIG. 7 shows differential protein expression, including metabolic and transporter proteins, in PDAC and non-malignant pancreatic cell lines. (A) Principal component analysis (PCA) on whole proteome (left panel), metabolism proteins (middle panel), and ‘SLC’ transporter proteins (right panel) expressed in PDAC (PANC1, CAPAN-I, HPAC) and non-malignant pancreatic (HPNE, hPSC #1) cell lines. (B) Relative protein expression across panel of non-malignant pancreatic and PDAC cell lines quantified by summing reporter ion counts of peptide-spectral matches for SLC1A5, SLC17A5, and SLC6A6. Error bars depict s.d. of two tandem mass tag-labeled biological replicates for each cell line.

FIG. 8 shows SLC38A2 is necessary for concentrative alanine influx in PDAC. (A) Immunoblot of deglycosylated SLC38A2 and N/K-ATPase in whole cell lysates (20 μg) extracted from pooled SLC38A2 knockout (sgSLC38A2 #1, #3) or control (sgTom) HY19636, MiaPaCa2, and PANC1 cells. Representative immunoblot depicted of three independent immunoblots. (B) Intracellular alanine levels normalized to 1×10⁶ cells in SLC38A2-deficient (sgSLC38A2 #1, #3) or control (sgTom) HY19636, MiaPaCa2, PANC1 cells cultured in DMEM or DMEM supplemented with 1 mM L-alanine for 24 hours. Error bars depict s.d. of three independent experiments. (C) Alanine carbon contribution to TCA cycle intermediates in SLC38A2-deficient (sgSLC38A2 #1, #3) or control (sgTom) HY19636, MiaPaCa2, and PANC1 cells cultured in DMEM supplemented with 1 mM ¹³C₃-alanine for 1 hour. Error bars depict s.d. of three independent experiments. (D) Intracellular alanine levels in SLC38A2-deficient (sgSlc38a2 #1) or control (sgTom) HY19636 cells cultured in DMEM or DMEM supplemented with 1 mM of either L-alanine or L-alanine tert-butyl ester for 24 hours. Error bars depict s.d. of three independent experiments. (E) Immunoblot of deglycosylated SLC38A2, non-deglycosylated SLC38A2, and actin in whole cell lysates (20 μg) extracted from PaTu-8988T control (sgTom), parental SLC38A2-deficient (sgSLC38A2 #1, −), and SLC38A2-deficient cells ectopically expressing empty vector (EV), sgRNA-resistant SLC38A2 (+SNAT2^{WT}), or mutant SLC38A2 incapable of binding to sodium (+SNAT2^{N82A}). Ectopically expressed SLC38A2 cDNAs, including non-functional mutant, are capable of forming mature protein evidenced by glycosylation capacity (lower panel). (F) Intracellular alanine levels in PaTu-8988T control (sgTom), SLC38A2-deficient (sgSLC38A2 #1; EV, SNAT2^{N82A}), and cDNA rescued (sgSLC38A2 #1; SNAT2^{WT}) cells cultured in DMEM for 24 hours. Error bars depict s.d. of three independent experiments.

FIG. 9 shows SLC38A2 loss in PDAC causes a metabolic crisis leading to decreased proliferative and clonogenic capacity. (A) Representative plates from clonogenic assay in SLC38A2-deficient (sgSLC38A2 #1, #3) and control (sgTom) HY19636, MiaPaCa2, and PANC1 cells cultured in DMEM for 7-10 days (HY19636, MiaPaCa2) or 14 days (PANC1). Imaged plates representative of three independent experiments. (B) Proliferation curve of control (sgTom, left panel) and SLC38A2-deficient (sgSlc38a2 #1, right panel) HY19636 cells cultured in DMEM or DMEM supplemented with 1 mM of either L-alanine or L-alanine tert-butyl ester over 120 hours. Data are plotted as cell proliferation relative to day 0 collected after cell attachment. Error bars depict s.d. of four independent experiments. (C) Total intracellular amino acid levels normalized to 1×10⁶ cells in SLC38A2-deficient (sgSLC38A2 #1, #3) or control (sgTom) 8902, PANC10.05, HY19636, 8988T, MiaPaCa2, or PANC1 cells cultured in DMEM for 24 hours. Error bars depict s.d. of three independent experiments. (D, E) Amino acid composition relative to control (sgTom) cells in SLC38A2-deficient (sgSLC38A2 #1, #3) HY19636, MiaPaCa2, PANC1, PANC10.05, PaTu-8902, and PaTu-8988T cells cultured in DMEM for 24 hours. Panels are labeled with corresponding cell line and error bars depict s.d. of three independent experiments. (F) Amino acid composition in PaTu-8988T control (sgTom), SLC38A2-deficient (sgSLC38A2 #1; EV, SNAT2^{N82A}), and cDNA rescued (sgSLC38A2 #1; SNAT2^{WT}) cells cultured in DMEM for 24 hours. Individual amino acid levels are normalized to control (sgTom) cells. Error bars depict s.d. of three independent experiments.

FIG. 10 shows reduced capacity to influx alanine drives amino acid crisis in SLC38A2-deficient PDAC cells. (A) Serine, glycine, proline, and glutamate extracellular fluxes in SLC38A2-deficient (sgSLC38A2 #1, #3) and control (sgTom) HY19636, MiaPaCa2, and PANC1 cells cultured in DMEM. Extracellular accumulation (+, secretion) or depletion (−, uptake) was measured in conditioned media over 24 hours and normalized to the viable cell density over the time course. Error bars depict s.d. of three independent experiments. (B) Essential amino acid (leucine, isoleucine, valine, threonine) uptake fluxes in SLC38A2-deficient (sgSLC38A2 #1, #3) and control (sgTom) HY19636, MiaPaCa2, and PANC1 cells cultured in DMEM for 24 hours. Error bars depict s.d. of three independent experiments. (C) Glutamine uptake flux in SLC38A2-deficient (sg #1, sg #3) and control (sgTom) HY19636, MiaPaCa2, and PANC1 cells cultured in DMEM for 24 hours. Error bars depict s.d. of three independent experiments. (D, E) Percent change in intracellular amino acids in control (sgTom, left panel) and SLC38A2-deficient (sgSlc38a2 #1, right panel) HY19636 cells cultured in DMEM of DMEM supplemented with 1 mM of either L-alanine or L-alanine tert-butyl ester for 24 hours. Data presented as a percent change for each amino acid relative to that measured in DMEM conditions. L-alanine or cell-permeable L-alanine tert-butyl ester failed to rescue amino acid defect in SLC38A2-deficient cells as evidenced by failure to increase amino acid levels relative to DMEM. Error bars depict s.d. of three independent experiments. (F) Immunoblot of deglycosylated SLC38A2, N/K-ATPase, and actin in whole cell lysates (20 μg) extracted from PANC1 pInducer-SLC38A2 cells cultured in DMEM supplemented±doxycycline (0.5 μg/mL) for 24 hours prior to addition of vehicle or MG-132 (10 μM) for 16 hours. (G) Immunoblot of deglycosylated SLC38A2, p(S51)-eIF2α, total eIF2α, LC3B, N/K-ATPase, and actin in whole cell lysates (20 μg) extracted from PANC1 pInducer-SLC38A2 control (sgTom) and SLC38A2-deficient (sgSLC38A2 #1) cells cultured in presence of doxycycline (0.5 μg/mL) or after wash-out of doxycycline for 24 hours. (H) Intracellular alanine levels normalized to 1×10⁶ cells in PANC1 pInducer-SLC38A2 control (sgTom) and SLC38A2-deficient (sgSLC38A2 #1) cells cultured in presence of doxycycline (0.5 μg/mL) or after wash-out of doxycycline for 24 hours. (I) Percent increase (+) or decrease (−) in intracellular amino acids in chronic SLC38A2-deficient (‘chronic’) and acute SLC38A2-deficient (‘acute’) cells cultured in DMEM after 12 hours acute doxycycline removal. Data are presented as a percent change relative to control PANC1 pInducer-SLC38A2 SLC38A2-deficient cells cultured in presence of doxycycline (0.5 μg/mL) in parallel for 12 hours. Error bars depict s.d. of three independent experiments.

FIG. 11 shows knockdown of SLC1A4 significantly suppresses alanine secretion and exchange in pancreatic stellate cells. (A) Immunoblot of SLC1A4, N/K-ATPase, and actin in whole cell lysates (30 μg) extracted from hPSC #1 (shGFP, shSLC1A4 #4) and mPSC (shGFP, shSlc1a3 #3) cultured in DMEM. (B) (left panel) Extracellular alanine label (M+3) dilution with unlabeled (M+0) alanine in SLC1A4 knockdown or control (shGFP) hPSC #1 cells cultured in DMEM supplemented with 1 mM ¹³C₃-alanine for 24 hours. Knockdown of SLC1A4 reduces the rate in alanine dilution. (right panel) Alanine secretion normalized to relative cell growth from control (shGFP) and SLC1A4 knockdown hPSC #1 cells cultured in DMEM for 24 hours. Error bars depict s.d. of three independent experiments. (C) Alanine secretion or exchange flux in SLC1A4 knockdown or control (shGFP) hPSC #1 cells cultured in DMEM or DMEM supplemented with 1 mM ¹³C₃-alanine. Net secretion flux calculated from molar secretion normalized to cell density over time. Exchange flux calculated from carbon turnover rate calculated from molar ¹³C₃-alanine dilution normalized to cell density over time. Error bars depict s.d. of three independent experiments. (D) Extracellular flux of SLC1A4 substrates including alanine, serine, and threonine in SLC1A4 knockdown and control (shGFP) hPSC #1 (left panel) and mPSC #1 (right panel) cells cultured in DMEM for 24 hours. Error bars depict s.d. of three independent experiments. (E) Cell proliferation relative to day 0 in SLC1A4 knockdown and control (shGFP) human and mouse stellate cell lines cultured in DMEM over 120 hours. Error bars depict s.d. of four independent experiments.

FIG. 12 shows SLC38A2 is highly expressed and plasma membrane localized in vivo and vital for PDAC tumor initiation and growth. (A) SLC38A2 staining in normal murine liver. Representative field from normal liver (4×; 10×, inset) are depicted with scale bars. Arrows indicate instances of punctate SLC38A2 in normal liver (right inset). (B) Representative live cell image of MDCK cells transiently transfected with 3 μg of SLC38A2-GFP (green) overnight and stained with MitoTracker (red) to visualize individual cells; scale bar indicated in figure. (C) Immunoblot of deglycosylated SLC38A2 and actin in whole cell lysates (20 μg) extracted from SLC38A2 knockdown and control (shGFP) PANC1 cells immediately prior to xenograft experiments. (D) Tumor initiation was significantly enhanced when PANC1 cells (2×10⁵) were co-injected with hPSC #1 cells (1×10⁶) in a subcutaneous xenograft model. Tumors were monitored bi-weekly by caliper measurement and considered if length and width were both ≥1 mm. (E) Tumor formation did not occur with injections of hPSC #1 cells (1×10⁶). (F) SLC38A2 knockdown and control (shGFP) PANC1 cells (2×10⁵) injected alone or hPSC #1 cells (1×10⁶) injected alone rarely formed tumors in a subcutaneous xenograft model. Tumors were monitored bi-weekly by caliper measurement and considered if length and width were both ≥1 mm. (G) Images of resected syngenic allograft and xenograft orthotopic tumors generated from injection of SLC38A2-deficient (sgSLC38A2 #1, #3) and control (sgTom) HY19636 cells (2.5×10⁴, top panel) or PANC1 cells (5×10⁵, bottom panel). Tumors were resected after 27 days (HY19636) or 63 days (PANC1). ‘X’ represents a mouse found dead prior to study endpoint. represents a pancreas without detectable tumor at study endpoint.

FIG. 13 shows (A) Western blot of Slc38a2 in HY15566 (mouse PDAC cells derived from KPC tumor). Loss of SLC38A2 leads to significantly reduced in vitro proliferation rate. (B) Dox-inducible cDNA fully rescues the proliferation defect in SLC38A2-deficient cells. Withdrawal of SLC38A2 by removing doxycycline immediately causes growth delay. Loss of SLC38A2 immediately suppresses alanine uptake and reduces intracellular alanine levels.

FIG. 14 shows (A) Injecting dox-controllable SLC38A2 cells subcutaneously into syngeneic C57BL\6J mice rescues tumor initiation at day 7 after injection (here referred to as day 0). (B) Withdrawal of doxycycline at day 0 (day 7 after injection) leads to a significant reduction in tumor growth in vivo, and doxycycline partially rescues the tumor burden at endpoint (day 25).

FIG. 15 shows SLC38A2 structure modeled based on homology to Aquifex aeolicus LeuTₐₐ (PDB ID: 3TT1). Modeled using I-TASSER from primary amino acid sequence for human SLC38A2 constrained using known structural elements. The sphere is predicted sodium binding location. Binding occurs between transmembrane domain (TMD) 1 and 8.

FIG. 16 shows putative alanine binding pocket identified by modeling the solvent accessible surface in PyMol using solvent radius of 1.4 Å (angstrom). Docking of alanine and known inhibitor α-(Methylamino) isobutyric acid, herein referred to as MeAIB, was performed within a 15×15×15 Å cube located 2 Å from the sodium atom. The center of the docking bounds is indicated by a red sphere (above).

FIG. 17 shows the model predicts favorable interactions (negative ΔG) between alanine and MeAIB within the predicted binding pocket.

FIG. 18 shows docking of >1500 FDA-approved compounds within 15×15×15 Å cube representing predicted alanine binding pocket. Negative binding energy plotted; larger deltaG represents more favorable interaction with SLC38A2 binding pocket. Significant enrichment of anti-psychotic and anti-depressant compounds, including SSRI, TCA, and 5-HT receptor agonists, indicated on plot.

FIG. 19 shows structures of relevant anti-psychotics and anti-depressants.

FIG. 20 shows predicted binding data for FVX.

FIG. 21 shows predicted binding data for FLX.

FIG. 22 shows predicted binding data for SRT.

FIG. 23 shows predicted binding data for PXT.

FIG. 24 shows predicted binding data for BNS.

FIG. 25 shows predicted binding data for SND.

FIG. 26 shows predicted interaction between the trifluorobenzyl moiety of fluoxetine (FLX) and phenylalanine 301 (Phe301) located in transmembrane domain 6 (TMD6) of SLC38A2.

FIG. 27 shows predicted interaction between the trifluorobenzyl moiety of fluvoxamine (FVX) and Tyrosine 94 (Tyr94) located in transmembrane domain 1 (TMD1) of SLC38A2.

FIG. 28 shows fluvoxamine treatment at 10 μM significantly and specifically inhibits alanine uptake in PDAC cells. (A) Alanine levels in culture media conditioned by 8988T cells treated with vehicle (DMSO) or fluvoxamine (FVX, 10 μM) over time. (B) Alanine uptake flux by 8988T cells treated with vehicle (DMSO) or fluvoxamine (FVX, 10 μM) for 96 hours. Data were normalized to cell growth over the treatment period. (C) Glutamine levels in culture media conditioned by 8988T cells treated with vehicle (DMSO) or fluvoxamine (FVX, 10 μM) over time. (D) Absolute values for amino acid uptake or secretion flux by 8988T cells treated with vehicle (DMSO) or fluvoxamine (FVX, 10 μM) for 96 hours. Data were normalized to cell growth over the treatment period.

FIG. 29 shows loss of SLC38A2 in cells results in 50% reduction in intracellular alanine levels (see panels A and B). Cells treated with vehicle, predicted inhibitors (FLX and PXT), or desipramine (TCA, not identified as inhibitor) significantly reduced intracellular alanine levels by ˜50% similar to genetic experiments (see C). (A) Western blot depicting SLC38A2 levels in PANC1 cells engineered to express SLC38A2 under control of doxycycline. Withdrawal of doxycycline results in a significant depletion in SLC38A2 levels after 24 hours. (C) Intracellular alanine levels in PANC1 cells treated with vehicle (DMSO), paroxetine (PXT; 10 μM), desipramine (DPM; 10 or fluoxetine (racemic FLX, 10 or 25 μM) for 24 hours.

FIG. 30 shows a scheme of the fluorescence assay.

FIG. 31 shows HyPer ratio plotted against time (minutes) at various concentrations of D-alanine.

FIG. 32 shows HyPer ratio plotted against time (minutes) of various concentrations of vehicle and MeAIB.

FIG. 33 shows HyPer ratio plotted against time (minutes) of various compounds.

FIG. 34 shows HyPer ratio plotted against time (minutes) of MeAIB and sertraline.

FIG. 35 shows intracellular alanine levels in MiaPaCa2 cells expressing HyPerDAAO cultured with 1 mM L-alanine and vehicle (veh.), MeAIB (2 mM), paroxetine (20 μM), or sertraline (20 μM) for 3 hours.

FIG. 36 shows percent inhibition in HyPer ratio of PXT, SRT, and MeAIB.

FIG. 37 shows SLC28A2 activity by measuring uptake of non-metabolizable alanine analog, methyl α-aminoisobutyrate (MeAIB). (A) Schematic depicting SLC38A2-dependent update of alanine structural analog, methyl α-aminoisobutyrate (MeAIB). In cells deficient in SLC38A2 expression, MeAIB uptake is significantly reduced. (B) Total ion chromatogram (TIC) of HY15549 dox-inducible SNAT2 cells treated with 1 mM MeAIB for 24 hours compared to vehicle (PBS). In cells lacking SLC38A2, MeAIB uptake, indicated by arrow, is significantly reduced. (C) Uptake of MeAIB follows Michaelis-Menten kinetics with an approximate Km of 3.5 mM. HY15549 dox-inducible SNAT2 cells were cultured with a concentration gradient of MeAIB for 15 minutes (gray circles) or 60 minutes (black squares). (D) Western blot depicting SLC38A2 expression relative to doxycycline after 24 hours culture in HY15549 dox-inducible SNAT2 cells. (E) Intracellular MeAIB levels in HY15549 dox-inducible cells cultured with dox (+SNAT2, left bar) and without dox (KO, right bar) for 24 hours with 1 mM MeAIB. (F) Intracellular MeAIB levels in HY15549 dox-inducible cells cultured with vehicle, paroxetine (PXT, 20 μM), or sertraline (SRT, 20 μm) for 24 hours with 300 μM MeAIB. (G) Schematic depicting inhibition of SLC38A2-dependent MeAIB uptake by sertraline (SRT) or paroxetine (PXT).

FIG. 38 shows intracellular MeAIB levels. Treatment with fluoxetine (FLX, 20 μm), fluvoxamine (FVX, 20 μm), paroxetine (PXT, 20 μm), or sertraline (SRT, 20 μm) inhibited MeAIB (0.3 mM) uptake over 1 hour compared to vehicle (PBS) treated HY15549 cells expressing dox-inducible SLC38A2/SNAT2.

FIG. 39 shows differentially expressed transporters between hPSC #1 and PANC1 cells.

### DETAILED DESCRIPTION OF THE DISCLOSURE

Although claimed subject matter will be described in terms of certain embodiments, other embodiments, including embodiments that do not provide all of the benefits and features set forth herein, are also within the scope of this disclosure. Various structural, logical, and process step changes may be made without departing from the scope of the disclosure.

Where a range of values is provided in this disclosure, it should be understood that all intervening ranges, and each intervening value, to the tenth of the value of the lower limit of the range and any other intervening value in that stated range is encompassed within the disclosure.

SLC38A2, one of the two neutral amino acid transporters, is highly expressed in pancreatic cancer cells relative to normal tissues and non-transformed cells within PDAC tumors. Genetically targeting SLC38A2 using RNAi or CRISPR/Cas9 in pancreatic cancer cells reveals that expression of SLC38A2 is required for alanine uptake, which is important for supporting PDAC metabolism. Pancreatic cancer cells lacking SLC38A2 fail to rewire their metabolism to compensate for loss of this transporter. Demonstrated herein is that SLC38A2 loss leads to an amino acid homeostatic crisis, which negatively impacts cell proliferation and tumor initiation and growth.

The present disclosure provides compositions and methods for interfering with uptake of neutral amino acids (e.g., alanine) in pancreatic cells. Alanine uptake can be inhibited by inhibiting the function and/or expression of SLC38A2 and/or SLC1A4.

In an aspect, the present disclosure provides compositions comprising inhibitors (e.g., compounds, antibodies, and the like) of alanine uptake. The compounds and/or antibodies in the compositions may inhibit the expression or function of SLC38A2 and/or SLC1A4 in a pancreatic cell (e.g., a pancreatic cancer cell, such as, for example, a pancreatic ductal adenocarcinoma cell). Non-limiting examples of inhibitors include small molecules (e.g., antidepressants), peptides and/or proteins (e.g., antibodies (an antigen binding fragment thereof or modification thereof)), RNA molecules (e.g., an interfering RNA (such as, for example, shRNA or siRNA) or dsRNA), and the like, and combinations thereof. The compositions may comprise one or more pharmaceutically acceptable carriers.

Non-limiting examples of antidepressants include one or more selective serotonin reuptake inhibitors (SSRI), one or more tricyclic antidepressants (TCA), one or more tetracyclic antidepressants (TeCA), one or more reversible inhibitors of monoamine oxidase-A (RIM-A), one or more 5-hydroxytryptamine receptor inhibitors (5-HTRi), or a combination thereof. Examples of SSRIs include, but are not limited to, fluvoxamine (FVX), fluoxetine (FLX), paroxetine (PXT), sertraline (SRT), and the like. Non-limiting examples of TCAs include amitriptyline and the like. Non-limiting examples of TeCAs include ciclopramine and the like.

Antibodies (including antigen binding fragments thereof or modifications thereof) can be directed to an epitope of SLC38A and/or SLC1A4.

The term “antibody” (or its plural form) as used herein encompasses whole antibody molecules, full-length immunoglobulin molecules, such as naturally occurring full-length immunoglobulin molecules or full-length immunoglobulin molecules formed by immunoglobulin gene fragment recombinatorial processes, as well as antibody fragments. Antibody fragments can be fragments comprising at least one antibody-antigen binding site. The term “antibody” includes e.g., monoclonal, polyclonal, multispecific (for example bispecific), recombinant, human, chimeric, and humanized antibodies. The term “antibody” also encompasses minibodies and diabodies, all of which preferably specifically inhibit SLC38A and/or SLC1A4. The term “antibody,” also encompasses immunoglobulins produced in vivo, in vitro, such as, for example, by a hybridoma, and produced by synthetic/recombinant means. An antibody may be modified by, for example, acetylation, formylation, amidation, phosphorylation, or polyethylene glycolation (PEGylation), as well as glycosylation. Antigen-binding fragments include, but are not limited to, Fab, F(ab′), F(ab′)2, Fv, dAb, Fd, CDR fragments, single-chain antibodies (scFv), bivalent single-chain antibodies, single-chain phage antibodies, diabodies, nanobodies and the like. The fragments of the antibodies may be produced synthetically or by enzymatic or chemical cleavage of intact immunoglobulins or may be genetically engineered by recombinant DNA techniques. These techniques are well known in the art. The antibodies useful for the present method may be obtained from a human or a non-human animal. For example, single domain antibodies or nanobodies produced by camelids can also be used. An antibody useful for the present method can be of any class. For example, an antibody of the present invention can be an antibody isotype IgG1, IgG2, IgG3, IgG4, IgM, IgA, IgD or IgE.

The compositions described herein may include one or more standard pharmaceutically acceptable carriers. Pharmaceutically acceptable carriers may be determined in part by the particular composition being administered, as well as by the particular method used to administer the composition. Accordingly, there are a wide variety of suitable formulations of pharmaceutical compositions of the present disclosure. Compounds may be freely suspended in a pharmaceutically acceptable carrier or the compounds may be encapsulated in liposomes and then suspended in a pharmaceutically acceptable carrier. Non-limiting examples of carriers include solutions, suspensions, emulsions, solid injectable compositions that are dissolved or suspended in a solvent before use, and the like. Injections may be prepared by dissolving, suspending, or emulsifying one or more of active ingredients in a diluent. Examples of diluents, include, but are not limited to distilled water for injection, physiological saline, vegetable oil, alcohol, dimethyl sulfoxide, and the like, and combinations thereof. Compositions may contain stabilizers, solubilizers, suspending agents, emulsifiers, soothing agents, buffers, preservatives, and the like, and combinations thereof. Compositions may be sterilized in the final formulation step or prepared by sterile procedure. A composition of the disclosure may also be formulated into a sterile solid preparation, for example, by freeze-drying, and may be used after sterilization or dissolution in sterile injectable water or other sterile diluent(s) immediately before use. Additional examples of pharmaceutically acceptable carriers include, but are not limited to, sugars, such as, for example, lactose, glucose, and sucrose; starches, such as, for example, corn starch and potato starch; cellulose, including sodium carboxymethyl cellulose, ethyl cellulose, and cellulose acetate; powdered tragacanth; malt; gelatin; talc; excipients, such as cocoa butter and suppository waxes; oils, such as, for example, peanut oil, cottonseed oil, safflower oil, sesame oil, olive oil, corn oil, and soybean oil; glycols, such as propylene glycol; polyols, such as glycerin, sorbitol, mannitol, and polyethylene glycol; esters, such as ethyl oleate and ethyl laurate; agar; buffering agents, such as, for example magnesium hydroxide and aluminum hydroxide; alginic acid; pyrogen-free water; isotonic saline; Ringer's solution; ethyl alcohol; phosphate buffer solutions; and other non-toxic compatible substances employed in pharmaceutical formulations. Additional non-limiting examples of pharmaceutically acceptable carriers can be found in: Remington: The Science and Practice of Pharmacy (2005) 21st Edition, Philadelphia, Pa. Lippincott Williams & Wilkins. Effective formulations include, but are not limited to, oral and nasal formulations, formulations for parenteral administration, and compositions formulated for with extended release. Parenteral administration includes infusions such as, for example, intramuscular, intravenous, intraarterial, intraperitoneal, subcutaneous administration, and the like.

Additional examples of compositions include, but are not limited to, (a) liquid solutions, such as, for example, an effective amount of a compound of the present disclosure suspended in diluents, such as, for example, water, saline or PEG 400; (b) capsules, sachets, depots or tablets, each containing a predetermined amount of the active ingredient, as liquids, solids, granules or gelatin; (c) suspensions in an appropriate liquid; and (d) suitable emulsions. The liquid solutions described above may be sterile solutions. Compositions may comprise, for example, one or more of lactose, sucrose, mannitol, sorbitol, calcium phosphates, corn starch, potato starch, microcrystalline cellulose, gelatin, colloidal silicon dioxide, talc, magnesium stearate, stearic acid, and other excipients, colorants, fillers, binders, diluents, buffering agents, moistening agents, preservatives, flavoring agents, dyes, disintegrating agents, and other pharmaceutically compatible carriers.

A composition may be in unit dosage form. In such form, the composition may be subdivided into unit doses containing appropriate quantities of the active component. The unit dosage form may be a packaged preparation, the package containing discrete quantities of preparation, such as, for example, packeted tablets, capsules, and powders in vials or ampoules. Also, the unit dosage form may be a capsule, tablet, cachet, or lozenge itself, or it may be the appropriate number of any of these in packaged form. The composition can, if desired, also contain other compatible therapeutic agents. The compositions may be used to deliver the compounds of the disclosure in a sustained release formulation.

In an aspect, the disclosure provides kits. A kit may comprise pharmaceutical preparations containing any one or any combination of compounds and printed material.

In various examples, a kit comprises a closed or sealed package that contains the pharmaceutical preparation. In various examples, the package comprises one or more closed or sealed vials, bottles, blister (bubble) packs, or any other suitable packaging for the sale, or distribution, or use of the compounds and compositions comprising compounds of the present disclosure. The printed material may include printed information. The printed information may be provided on a label, or on a paper insert, or printed on the packaging material itself. The printed information may include information that identifies the compound in the package, the amounts and types of other active and/or inactive ingredients, and instructions for taking the composition, such as the number of doses to take over a given period of time, and/or information directed to a pharmacist and/or another health care provider, such as a physician, or a patient. The printed material may include an indication that the pharmaceutical composition and/or any other agent provided with it is for treatment of a subject having cancer and/or other diseases and/or any disorder associated with cancer and/or other diseases. In various examples, the product includes a label describing the contents of the container and providing indications and/or instructions regarding use of the contents of the container to treat a subject having any cancer and/or other diseases. A kit may comprise a single dose or multiple doses.

In an aspect, the present disclosure provides methods of treating pancreatic cancer (e.g., pancreatic ductal adenocarcinoma). Various examples comprise using one or more inhibitors or compositions thereof.

A method may comprise inhibiting SLC38A2 and/or SLC1A4 in a pancreatic cell (e.g., pancreatic cancer cell, such as, for example, a pancreatic ductal adenocarcinoma cell). Inhibiting SLC38A2 and/or SLC1A4 may inhibit alanine uptake in a pancreatic cell (e.g., pancreatic cancer cell, such as, for example, a pancreatic ductal adenocarcinoma cell). Various other examples comprise genetic modification of a pancreatic cancer cell (e.g., pancreatic ductal adenocarcinoma cell). In an example, a method of the present disclosure for treating pancreatic cancer comprises administering to an individual in need of treatment a composition of the present disclosure.

In various examples, a method comprises contacting a cell with one or more SSRIs, one or more TCAs, one or more TeCAs, one or more RIM-As, one or more 5-HTRis, or a combination thereof. Contacting that cell may inhibit alanine uptake in a pancreatic cancer cell and/or inhibit the growth of pancreatic cells (e.g., pancreatic cancer cells, such as, for example, pancreatic ductal adenocarcinoma cells). For example, the method may inhibit the expression and/or function of SLC38A2 and/or SLC1A4.

Expression of SLC38A2 and/or SLC1A4 can be down regulated by methods known in the art. For example, RNAi-mediated reduction in SLC38A2 and/or SLC1A4 mRNA may be carried out. RNAi-based inhibition can be achieved using any suitable RNA polynucleotide that is targeted to SLC38A2 and/or SLC1A4 mRNA. In embodiments, a single stranded or double stranded RNA, wherein at least one strand is complementary to the target mRNA, can be introduced into the cell to promote RNAi-based degradation of target mRNA. In another embodiment, microRNA (miRNA) targeted to the SLC38A2 and/or SLC1A4 mRNA can be used. In another embodiment, a ribozyme that can specifically cleave SLC38A2 and/or SLC1A4 mRNA can be used. In another embodiment, small interfering RNA (siRNA) can be used. siRNA can be introduced directly, for example, as a double stranded siRNA complex, or by using a modified expression vector, such as a lentiviral vector, to produce an shRNA. As is known in the art, shRNAs adopt a typical hairpin secondary structure that contains a paired sense and antisense portion, and a short loop sequence between the paired sense and antisense portions. shRNA is delivered to the cytoplasm where it is processed by DICER into siRNAs. siRNA is recognized by RNA-induced silencing complex (RISC), and once incorporated into RISC, siRNAs facilitate cleavage and degradation of targeted mRNA. Generally, shRNA polynucleotide used to suppress SLC38A2 and/or SLC1A4 mRNA expression can comprise or consist of between 45-100 nucleotides, including all nucleotide values and ranges therebetween. For delivering siRNA via shRNA, modified lentiviral vectors can be made and used according to standard techniques, given the benefit of the present disclosure. Custom siRNAs or shRNA can be obtained from, for example Thermo-Dharmacon or Cellecta for transient transfection resulting in temporary reduction in the targeted mRNA levels. The lentiviruses are capable of stably and permanently infecting target cells, such as by integrating into a genome of a cell.

In another aspect, the disclosure includes disrupting the target gene such that SLC38A2 and/or SLC1A4 mRNA and protein are not expressed. In one embodiment, the SLC38A2 and/or SLC1A4 gene can be disrupted by targeted mutagenesis. In embodiments, targeted mutagenesis can be achieved by, for example, targeting a CRISPR (clustered regularly interspaced short palindromic repeats) site in the target gene. So-called CRISPR systems designed for targeting specific genomic sequences are known in the art and can be adapted to disrupt the target gene for making modified cells encompassed by this disclosure. In general, the CRISPR system includes one or more expression vectors encoding at least a targeting RNA and a polynucleotide sequence encoding a CRISPR-associated nuclease, such as Cas9, but other Cas nucleases can alternatively be used. CRISPR systems for targeted disruption of mammalian chromosomal sequences are commercially available.

The compositions and components thereof (e.g., compounds, antibodies, RNA molecules, and the like) may be suitable in methods to treat pancreatic cancer in an individual diagnosed with pancreatic cancer, such as, for example, pancreatic ductal adenocarcinoma.

The methods of the present disclosure may be used in combination with other methods of treating pancreatic cancer. In various examples, the methods of the present disclosure may be used in combination with resection of a pancreatic tumor.

In an example, one or more compounds and/or one or more composition comprising one or more compounds described herein may be administered to a subject in need of treatment using any known method and/or route, including oral, parenteral, subcutaneous, intraperitoneal, intrapulmonary, intranasal and intracranial injections. Parenteral infusions include intramuscular, intravenous, intraarterial, intraperitoneal, and subcutaneous administration. The present disclosure also provides topical and/or transdermal administration.

In an example, a compound is used to inhibit alanine uptake. In an example, a method comprises administering to an subject in need of treatment with a composition comprising an inhibitor in an amount (e.g., 0.1 nM to 100 mM, including all 0.1 nM values and ranges therebetween (e.g., 1 nM to 20 nM, 1 nM to 50 nM, 1 nM to 100 nM, 1 nM to 250 nM, 1 nM to 500 nM, 1 nM to 750 nM, 1 nM to 1 mM, 5 nM to 20 nM, 5 nM to 50 nM, 5 nM to 100 nM, 5 nM to 250 nM, 5 nM to 500 nM, 5 mM to 1 mM, 10 nM to 50 nM, 10 nM to 100 nM, 25 nM to 50 nM, 25 nM to 100 nM, 25 nM to 250 nM, 25 nM to 500 nM)) and time sufficient to inhibit Ala uptake and/or inhibition of the expression and/or function of SLC38A2 and/or SLC1A4.

A method can be carried out in an individual in need of treatment who has been diagnosed with or is suspected of having pancreatic cancer. A method can also be carried out in a subject who has a relapse or a high risk of relapse after being treated for pancreatic cancer.

An individual in need of treatment may be a human or non-human mammal. Non-limiting examples of non-human mammals include cows, pigs, mice, rats, rabbits, cats, dogs, other agricultural animal, pet, service animals, and the like.

In an aspect, the present disclosure provides methods for identifying whether a tumor (e.g., a pancreatic tumor) is cancerous or non-cancerous.

A method of identifying a tumor (e.g., pancreatic tumor) as cancerous or non-cancerous can comprise obtaining a sample of cells from the tumor (e.g., pancreatic cells from the pancreatic tumor) and identifying the location of SLC38A2 protein in the cells. If SLC38A2 protein is localized in the plasma membrane, the tumor is identified as cancerous. If the intracellular localization of SLC38A2 is in a non-plasma membrane domain, the tumor is identified as non-cancerous.

Localization of SLC38A2 may be identified by using an antibody (including an antigen binding fragment thereof or modification thereof). The antibody (or fragment or modification thereof) may be detectably labeled. Various detectable labels are known in the art. The identifying may further comprise imaging. Various imaging methods are known in the art.

In an aspect, the present disclosure provides methods of identifying inhibitors for SLC38A2 and/or SLC1A4 and inhibiting alanine uptake. Methods may be experimental and/or in silico.

A method of identifying an inhibitor of SLC38A2 and/or SLC1A4 may comprise determining the alanine uptake in a pancreatic cell (e.g., a pancreatic cancer cell) in the presence or absence of a candidate inhibitor. Without intending to be bound by any particular theory, it is considered that a decrease in alanine uptake in the presence of the candidate inhibitor compared to a control is indicative of a desirable (e.g., suitable) inhibitor of SLC38A2 and/or SLC1A4.

A method of identifying an inhibitor of alanine uptake may comprise performing a first measurement of alanine concentration in a cellular media comprise pancreatic cells (e.g., pancreatic cancer cells); contacting the pancreatic cells (e.g., pancreatic cancer cells) with the inhibitor; performing a second measurement of alanine concentration in the cellular media; and determining if there is a change in alanine concentration between the first and second measurements. An increase of alanine concentration in the cellular media correlated to an inhibition of alanine uptake and the compound is identified as inhibiting alanine uptake via the increase of alanine concentration in the cellular media.

In silico methods may be used alone or in combination with other methods to determine if a compound is a suitable inhibitor of SLC38A2 and/or SLC1A4. Such a method may be in silico molecular docking analysis to detect a desirable binding energy. For example, a desirable binding energy may be −1 to −10 kcal/mol, including every 0.01 kcal/mol value and range therebetween.

The following Statements may be non-limiting examples of the present disclosure.

Statement 1. A method of inhibiting SLC38A2 in a pancreatic cell, comprising contacting the cell with an inhibitor of the expression or function of SLC38A2.
Statement 2. A method of inhibiting alanine uptake by a pancreatic cell, comprising contacting the cell with an inhibitor of the expression or function of SLC38A2.
Statement 3. A method of inhibiting the growth of pancreatic cells, comprising contacting the cells or introducing into the cells an inhibitor of SLC38A2, where the inhibitor is capable of inhibiting the expression or function of SLC38A2 in the cells.
Statement 4. The method according to any one of the preceding Statements, where the pancreatic cell(s) is/are pancreatic cancer cell(s).
Statement 5. The method according to Statement 4, where the pancreatic cancer cell(s) is/are pancreatic ductal adenocarcinoma cells.
Statement 6. A method of treating pancreatic cancer, comprising administering to an individual in need of treatment a composition comprising an inhibitor of SLC38A2, where the inhibitor is capable of inhibiting the expression or function of SLC38A2 in a pancreatic cancer cell.
Statement 7. A method according to any one of the preceding Statements, where the SLC38A2 inhibitor is an antidepressant.
Statement 8. A method according to Statement 7, where the antidepressant is a serotonin reuptake inhibitor (S SRI), a tricyclic antidepressant (TCA), a tetracyclic antidepressant (TeCA), serotonin norepinephrine reuptake inhibitor (SNRI), a reversible inhibitor of monoamine oxidase-A (RIM-A), a 5-hydroxytryptamine receptor inhibitor (5-HTRi), or a combination thereof.
Statement 9. A method according to Statement 8, where the antidepressant is an SSRI chosen from fluvoxamine, fluoxetine, paroxetine, sertraline, and the like, and combinations thereof.
Statement 10. A method according to Statement 8, where the antidepressant is the TCA amitriptyline.
Statement 11. A method according to Statement 8, where the antidepressant is the TeCA ciclopramine.
Statement 12. A method according to any one of Statements 7-11, where the dosage of the antidepressant is 1 nM to 100 mM, including all 0.1 nM values and ranges therebetween (e.g., 1 nM to 20 nM, 1 nM to 50 nM, 1 nM to 100 nM, 1 nM to 250 nM, 1 nM to 500 nM, 1 nM to 750 nM, 1 nM to 1 mM, 5 nM to 20 nM, 5 nM to 50 nM, 5 nM to 100 nM, 5 nM to 250 nM, 5 nM to 500 nM, 5 mM to 1 mM, 10 nM to 50 nM, 10 nM to 100 nM, 25 nM to 50 nM, 25 nM to 100 nM, 25 nM to 250 nM, 25 nM to 500 nM).
Statement 13. A method according to any one of Statements 1-6, where the SLC38A2 inhibitor is an antibody (including an antigen binding fragment thereof, or a modification thereof) directed to an epitope of SLC38A2 protein.
Statement 14. A method according to any one of Statements 1-6, where the SLC38A2 inhibitor is an interfering RNA (RNAi) molecule or a dsRNA.
Statement 15. A method according to Statement 14, where the RNAi molecule is shRNA or siRNA.
Statement 16. A method according to any one of Statements 1-6, where SLC38A2 is inhibited by disruption of a sequence encoding SLC38A2 (e.g., via CRISPR).
Statement 17. A method according to Statement 6, where the individual has been diagnosed with pancreatic cancer.
Statement 18. A method according to Statement 17, where the pancreatic cancer is pancreatic ductal adenocarcinoma.
Statement 19. A method according to Statements 6, 17, or 18, where the method is used in combination with resection of the pancreatic tumor.
Statement 20. A method according to Statement 6, wherein the composition administered to the individual comprises the antidepressant at a concentration of 1 nM to 100 mM, including every 0.1 nM value and range therebetween, and is administered for 1 to several days.
Statement 21. A method of identifying whether a pancreatic tumor is cancerous or non-cancerous, comprising: obtaining a sample of pancreatic cells from the pancreatic tumor; and identifying the location of the SLC38A2 protein in the cells; where localization of the SLC38A2 protein in the plasma membrane is indicative of a cancerous tumor and intracellular localization of SLC38A2 in non-plasma membrane domains is indicative of a non-cancerous tumor.
Statement 22. A method according to Statement 21, where localization of SLC38A2 is identified by using an antibody (including an antigen binding fragment thereof, or a modification thereof) directed to an epitope of the SLC38A2 protein.
Statement 23. A method according to Statement 22, where the antibody of a fragment or modification thereof is detectably labeled.
Statement 24. A method according to any one of Statements 21-23, further comprising treating an individual having the pancreatic tumor that is cancerous.
Statement 25. A method according to any one of Statements 21-24, where the identifying step comprises imaging.
Statement 26. A method of identifying an inhibitor of SLC38A2, comprising: determining cellular localization of SLC38A2 protein in a pancreatic cell in the presence and absence of a candidate inhibitor, where a decrease in localization of SLC38A2 protein in the plasma membrane compartment in the presence of the candidate inhibitor compared to a control is indicative of a suitable inhibitor of SLC38A2.
Statement 27. A method according to Statement 26, where the determining step comprises imaging.
Statement 28. A method according to Statements 26 or 27, where the control does not comprise an inhibitor or the candidate inhibitor.
Statement 29. A method of identifying an inhibitor of SLC38A2, comprising: determining alanine uptake in a pancreatic cell in the presence or absence of a candidate inhibitor, wherein a decrease in alanine uptake in the presence of the candidate inhibitor compared to a control is indicative of a suitable inhibitor of SLC38A2.
Statement 30. A method according to Statement 29, where the control does not comprise an inhibitor or the candidate inhibitor.
Statement 31. A method for identifying a compound that inhibits alanine uptake in pancreatic cells, comprising: performing a first measurement of alanine concentration in a cellular media comprising pancreatic cells; contacting the pancreatic cells with the compound; performing a second measurement of alanine concentration in the cellular media comprising pancreatic cells; and determining if there is a change in the alanine concentration of the cellular media, where an increase of alanine concentration in the cellular media correlates to an inhibition of alanine uptake and the compound is identified as inhibiting alanine uptake via the increase of alanine concentration in the cellular media.
Statement 32. A method according to any one of Statements 26-31, where the pancreatic cell(s) is/are pancreatic cancer cell(s).
Statement 33. A method according to Statement 32, where the pancreatic cancer cell(s) is/are pancreatic ductal adenocarcinoma cell(s).
Statement 34. A method according to any one of Statements 26-33, where a binding energy of a candidate inhibitor to SLC38A2 is determined via in silico molecular docking analysis.
Statements 35. A method according to Statement 34, where a binding energy of −1 to −10 kcal/mol indicates a suitable candidate inhibitor.
Statement 36. A method of inhibiting SLC1A4 in a pancreatic cell, comprising contacting the cell with an inhibitor of the expression or function of SLC1A4.

The following examples are presented to illustrate the present disclosure. They are not intended to be limiting in any matter.

### Example 1

This example illustrates methods of the present disclosure.

Described herein is that alanine crosstalk between PSCs and PDAC is orchestrated by differential expression of the neutral amino transporters SLC1A4 and SLC38A2. Specifically, it is described herein that PSCs utilize SLC1A4 to rapidly exchange alanine to maintain extracellular concentrations at levels observed in human and murine PDAC tumors. Moreover, it was demonstrated that alanine uptake in PDAC requires SLC38A2. Cells lacking SLC38A2 fail to concentrate alanine and undergo a metabolic crisis reducing proliferative capacity in vitro and in vivo. These results demonstrate that stromal-cancer metabolic niches can form through differential expression of transporters and that disrupting nutrient exchange through transporter inhibition represents a previously unappreciated means of targeting the metabolic demands of pancreatic cancer.

Results.

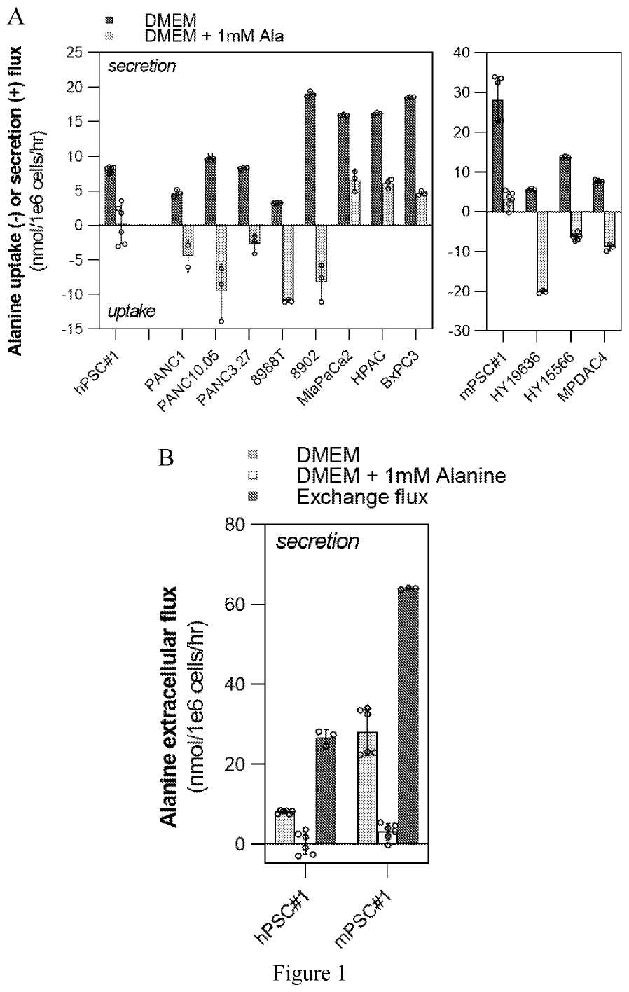

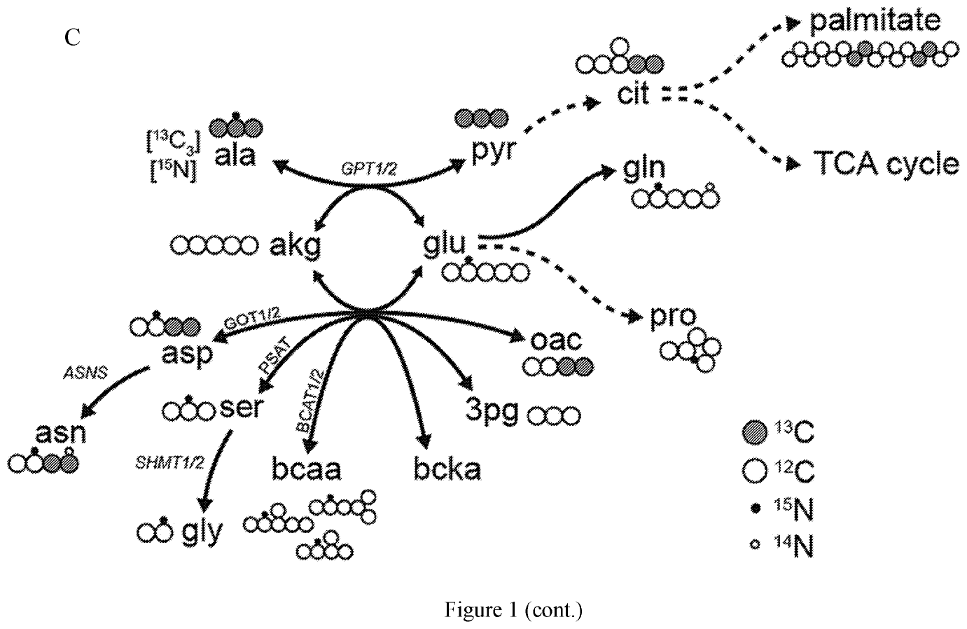

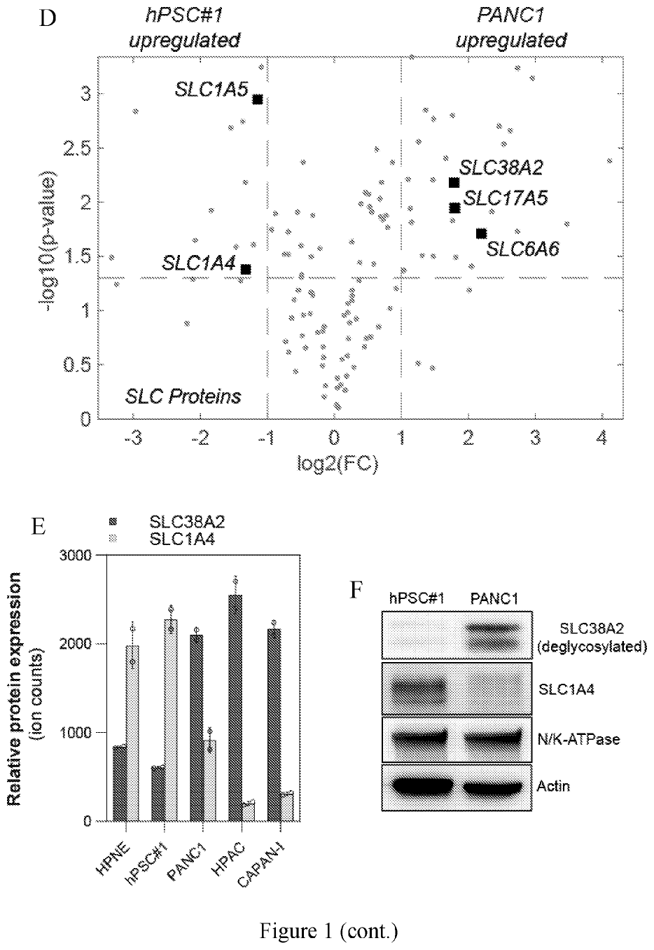

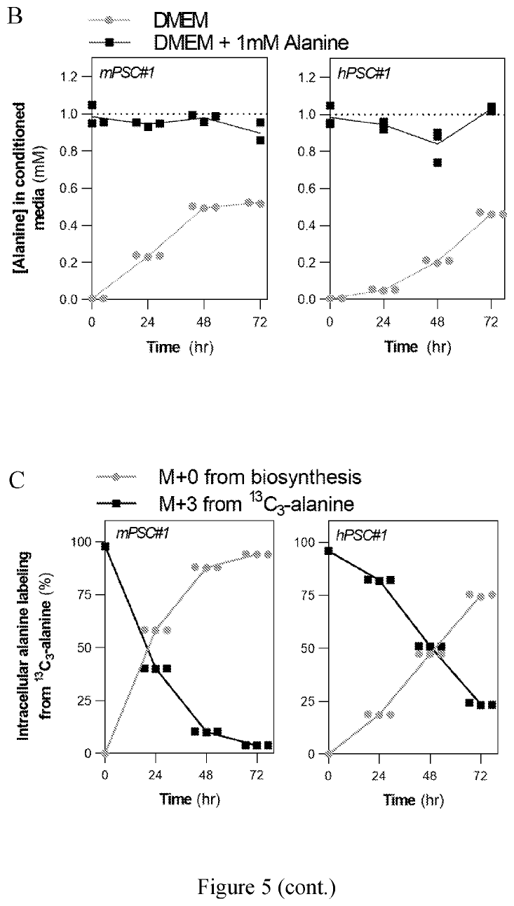

It was previously demonstrated there is a cooperative crosstalk of alanine between pancreatic stellate cells (PSCs) and PDAC, which highlighted a metabolic support role of the desmoplastic stromal response observed in pancreatic tumors. However, the mechanistic basis of how cooperative nutrient sharing occurs and the metabolic consequences of selectively disrupting this cross-talk were unexplored. The uptake and secretion fluxes of alanine and other amino acids were measured in a panel of human and mouse PDAC cell lines. The majority of PDAC cell lines significantly influx alanine and PSCs primarily efflux alanine (FIG. 1a). As DMEM does not contain alanine, it was observed net secretion in all cell lines cultured in basal media suggesting that mass action drives efflux in alanine austere environments (FIG. 1a). Notably, alanine influx was of similar magnitude to serine and glycine flux, amino acids reported to be critical for cancer cell proliferation (FIG. 5a). In PSCs, net alanine efflux was completely inhibited upon exogenous alanine supplementation (FIG. 1a,b and FIG. 5b); however, extracellular alanine carbon was rapidly exchanged at a rate ˜3× higher than the net secretion flux resulting in substantial dilution of ¹³C₃-alanine in the media with unlabeled alanine over time (FIG. 1b and FIG. 5c). These data suggest that PSCs are programmed to rapidly exchange alanine to maintain TME alanine concentrations at 1 mM, in agreement with reported alanine concentrations measured in human and murine PDAC tumors.

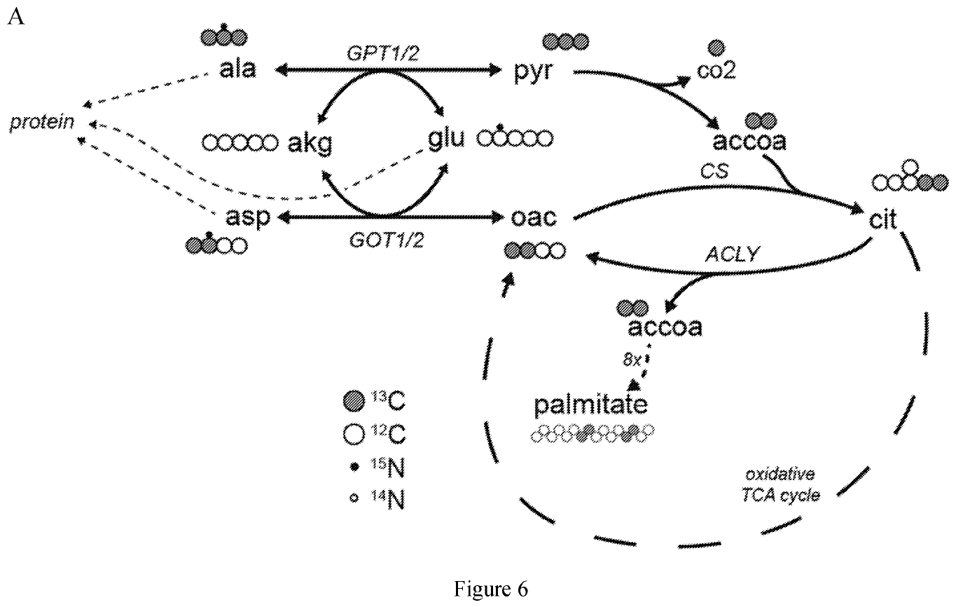

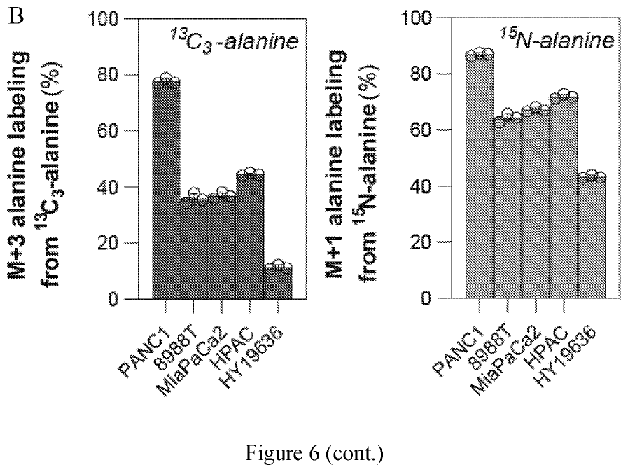

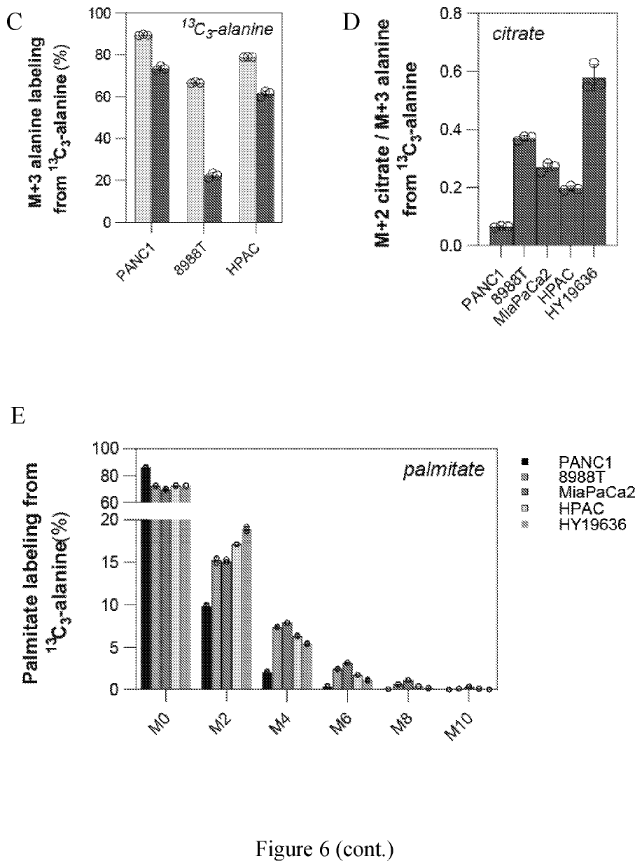

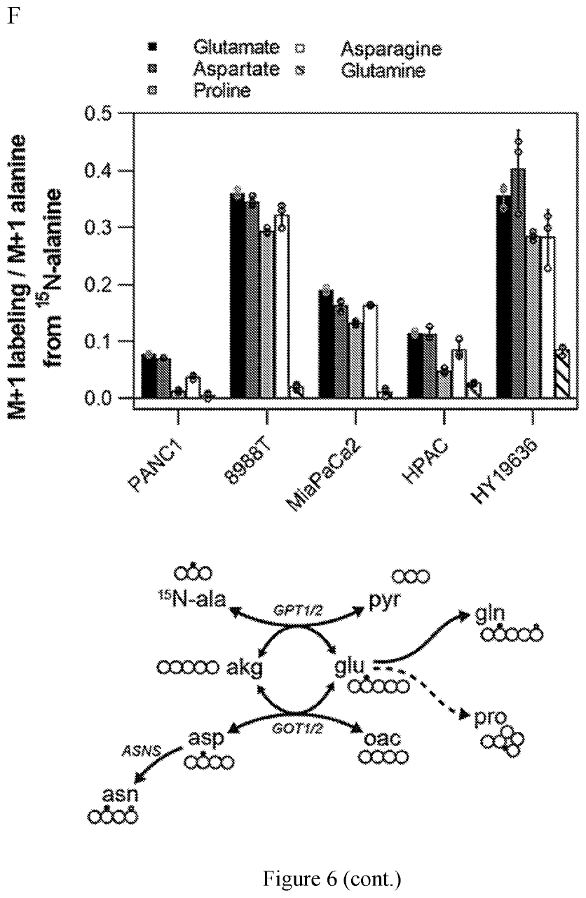

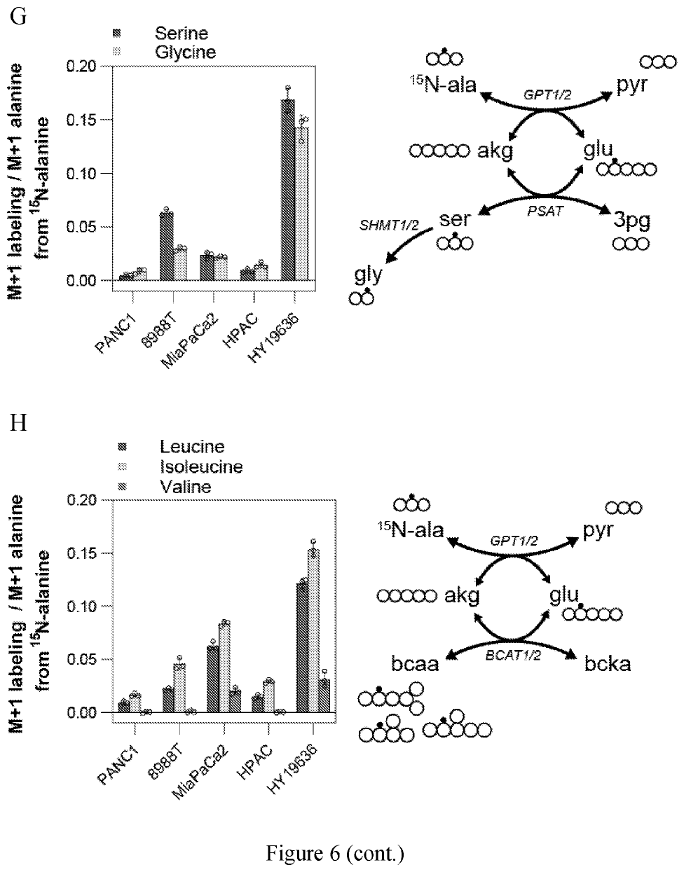

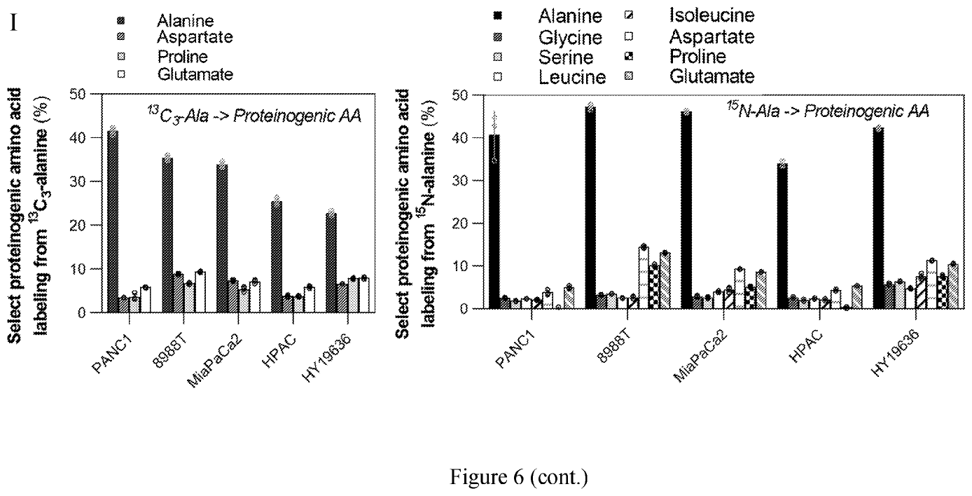

To determine if alanine was fueling specific metabolic pathways in PDAC cells, stable-isotope tracing was performed using uniformly ¹³C- or ¹⁵N-labeled alanine and measured incorporation into biosynthetic and central carbon metabolites or transamination products. Significant contribution of alanine-derived carbon and nitrogen was measured in proteinogenic amino acids, TCA intermediates, de novo synthesized fatty acids, and products of transamination pathways (FIG. 1c and FIG. 6), suggesting that alanine contributes significantly to intracellular bioenergetic and anabolic pathways in PDAC. However, alanine utilization was heterogeneous across the cell panel and did not correlate with extracellular flux directionality. Furthermore, intracellular alanine labeling from ¹³C- or ¹⁵N-label failed to reach isotopic steady-state and gradually decreased over time (FIG. 6c), suggesting that PDAC cells channel alanine to distinct subcellular compartments for consumption and synthesis. These data demonstrate how certain PDAC cell lines can maintain high ¹³C incorporation into mitochondrial TCA intermediates (e.g., MiaPaCa2) yet efflux alanine, presumably from distinct, unlabeled subcellular pool (FIG. 1a, c and FIG. 6).

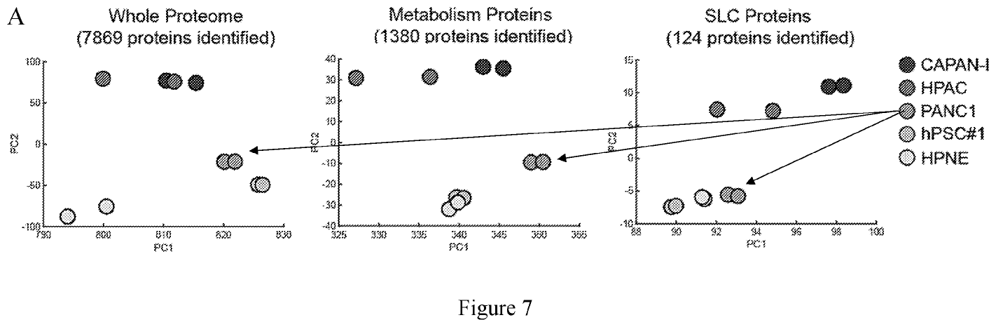

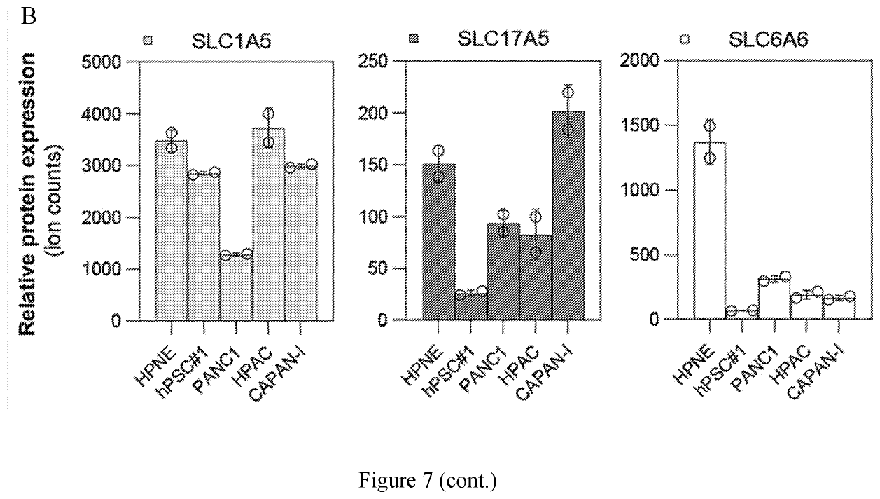

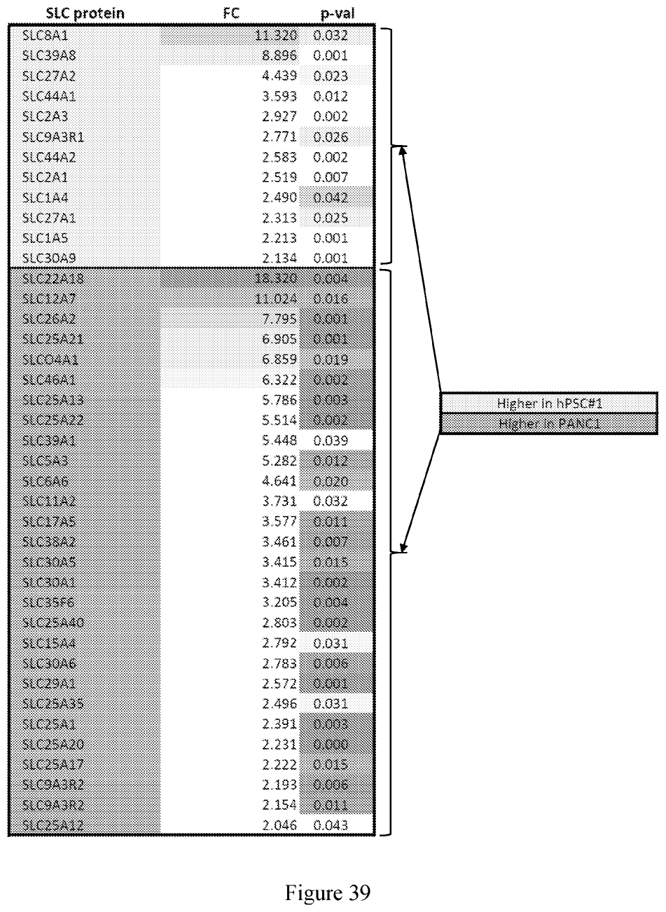

It was determined that targeting downstream alanine utilization may be challenging and vulnerable to metabolic rewiring; thus, it was sought to better understand how alanine crosstalk between PSC-PDAC occurs at the level of transport. It was determined that unique transport mechanisms must exist to facilitate alanine exchange within the PSC-PDAC niche. Importantly, flux and tracing results suggest that alanine transport in PSC and PDAC may be driven by distinct mechanisms. To identify candidate transporters involved in PSC-PDAC alanine crosstalk, a quantitative proteomics dataset in a panel of PDAC and PSC cell lines was analyzed. Principal component analysis (PCA) on the whole proteome or protein subsets involved in metabolic processes or transport revealed divergent expression profiles in stromal and PDAC cell lines (FIG. 7a). SLC proteins differentially expressed between PSCs and a commonly used human PDAC line were analyzed and identified 40 proteins which, excluding mitochondrial (SLC25) and non-amino acid transporters, reduced to five candidates (FIG. 1d and FIG. 39). Further exclusion of transporters depleted or expressed in only a single cell line identified two plasma membrane-localized neutral amino acid transporters—SLC1A4 and SLC38A2—that were highly expressed in either stromal or PDAC cells, respectively (FIG. 1e-f and FIG. 7b). L-alanine is a reported substrate for both SLC1A4 and SLC38A2 indicating differential transporter expression in PSC and PDAC may facilitate alanine crosstalk.

The SLC superfamily comprises an estimated 456 genes and pseudogenes classified into 52 subfamilies. Approximately 25% of SLCs are thought to be involved in the transport of amino acids, including members of the SLC1 and SLC38 families. The SLC1 family contains seven members consisting of five high-affinity charged amino acid transporters involved in neurotransmitter transport (SLC1A1-3, SLC1A6, SLC1A7) and two sodium-dependent neutral amino acid transporters (SLC1A4, SLC1A5). SLC1A5/ASCT2 has been the focus of several studies in multiple cancer types with broad spectrum amino acid specificities, including glutamine. Given the importance of glutamine to PDAC and proliferating cells in vitro, similar levels of SLC1A5 and significant glutamine uptake was observed across the cell panel (FIG. 5a). In contrast, SLC1A4/ASCT1 has attracted far less attention beyond its initial cloning and characterization in X. laevis oocytes as an alanine, serine, cysteine, and threonine transporter and association with inborn errors of metabolism affecting neural development. SLC38A2/SNAT2 was initially characterized as an electrogenic sodium-neutral amino acid co-transporter with a preference for alanine and other small neutral amino acids. While several transporters are biochemically characterized to transport alanine, it is unclear from biochemical studies which transporters drive context-dependent alanine transport in intact PSC and PDAC cells.

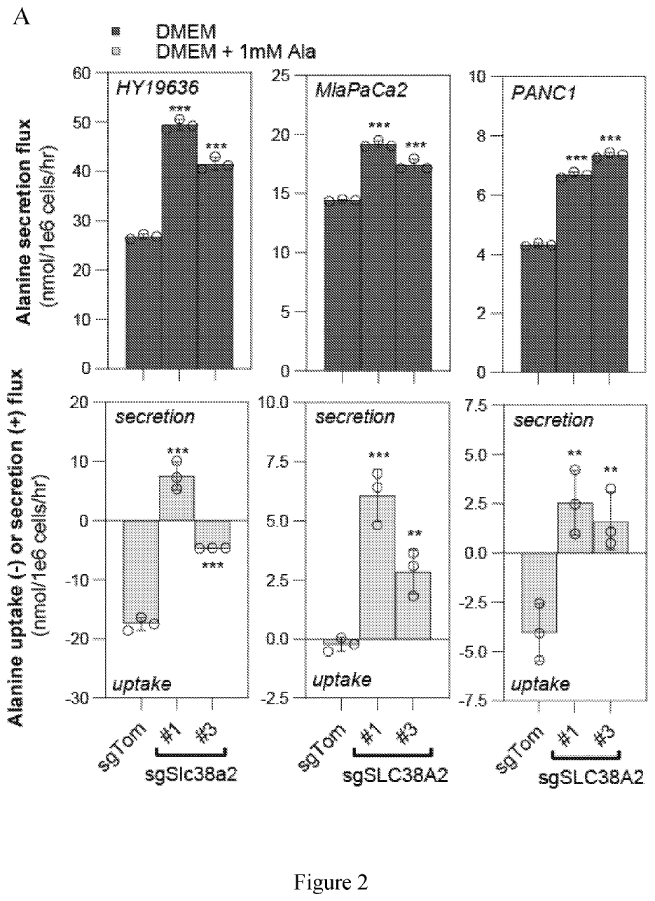

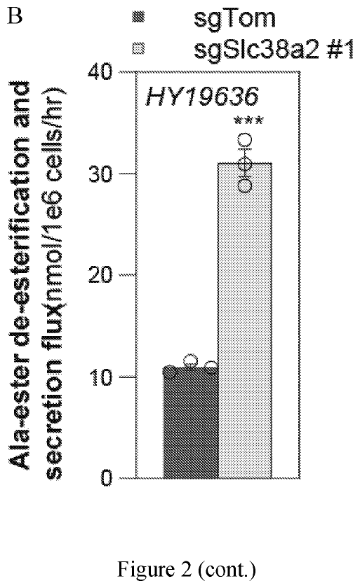

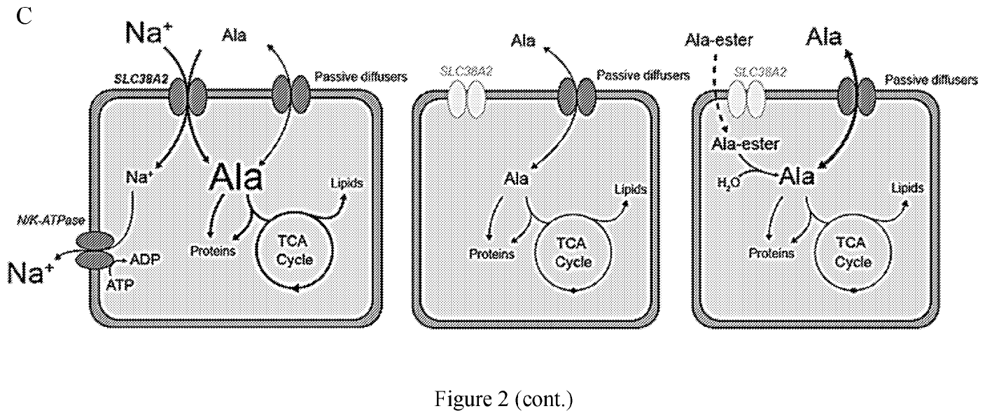

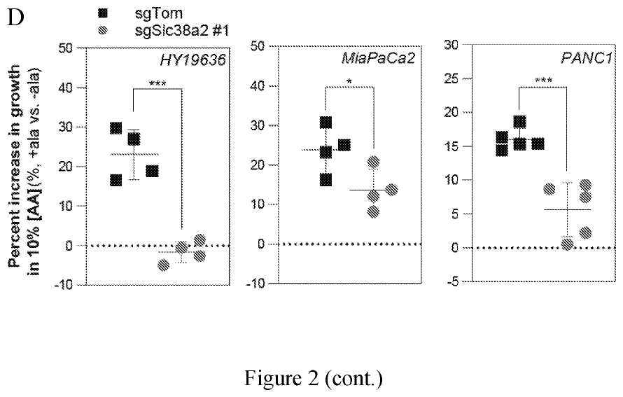

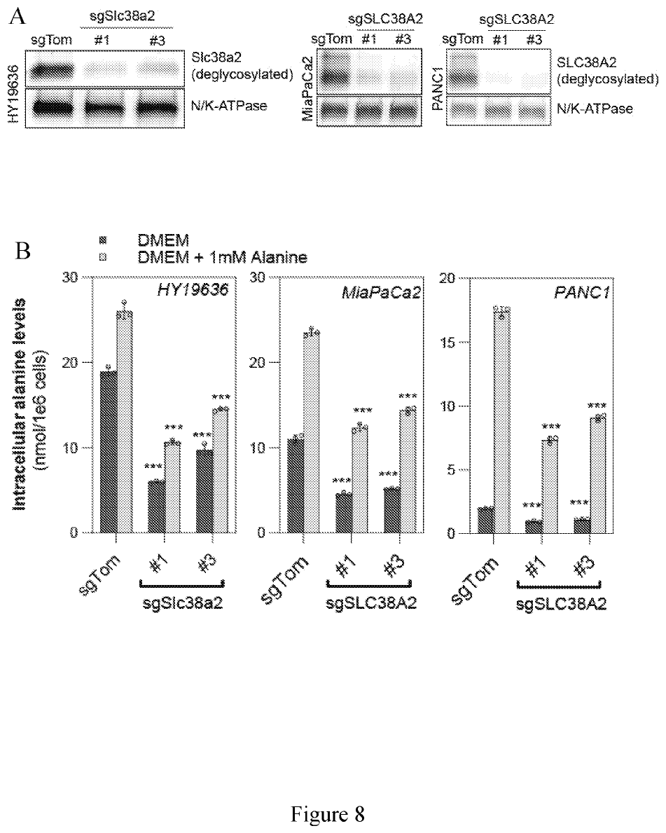

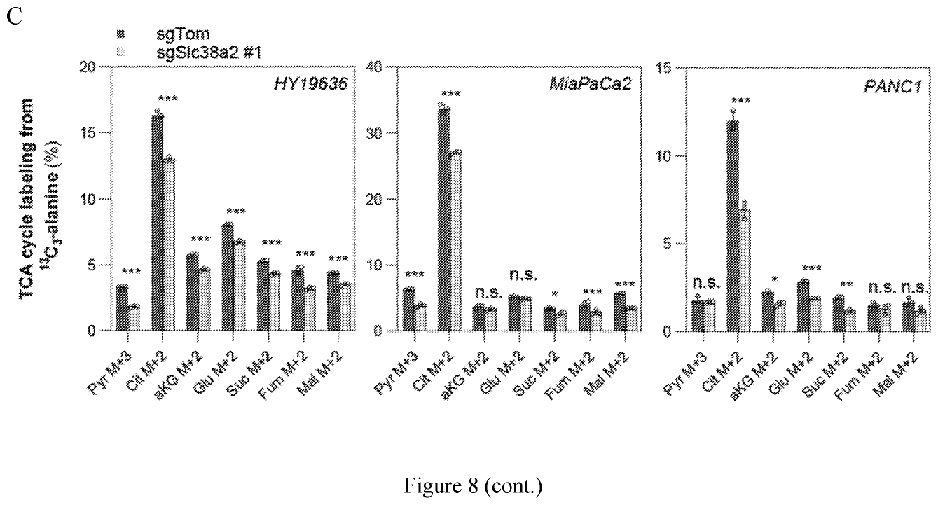

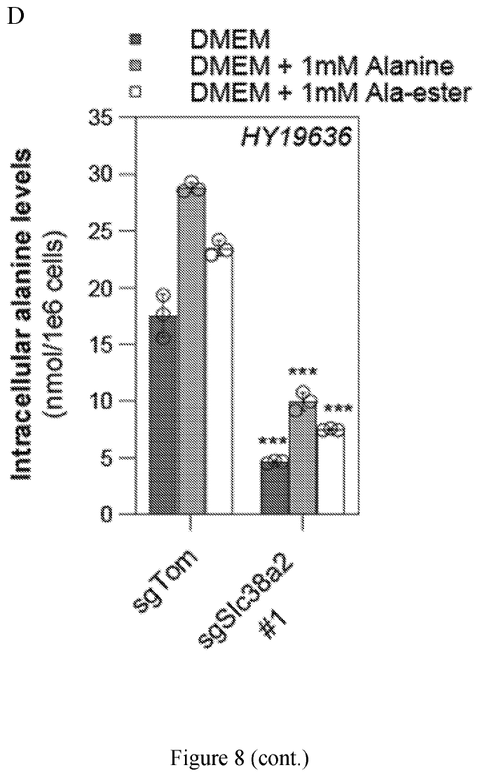

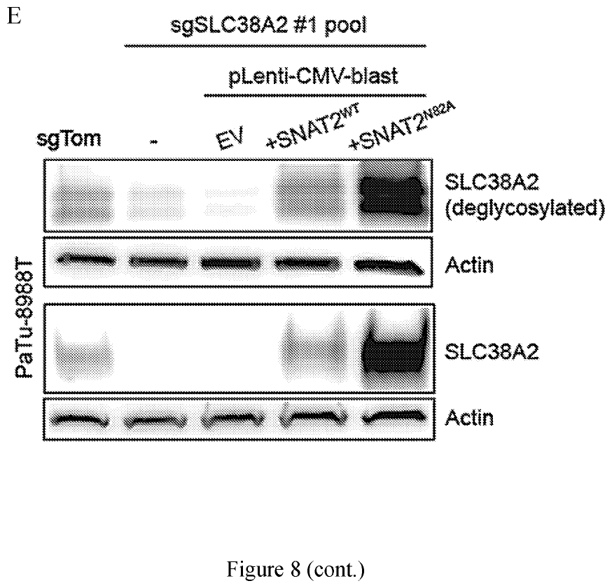

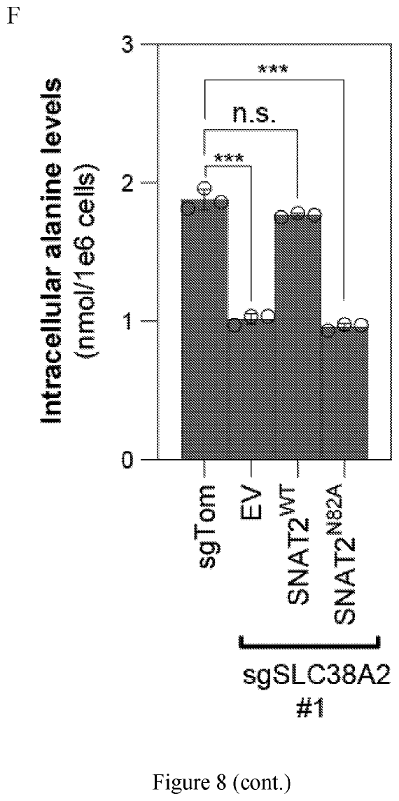

While metabolic studies of cultured cells have limitations, given the lack of a physiologically-relevant stromal and nutrient microenvironment, they nonetheless have proven indispensable to the study of intracellular metabolism of various cancers and provide a tractable system to dissect complex metabolic machinery, including nutrient transport. It was first sought to understand the impact of loss of either SLC1A4 or SLC38A2 in intact pancreatic stellate and PDAC cells, respectively, through CRISPR/Cas9- or RNAi-mediated loss-of-function and metabolic flux studies to understand their contribution to alanine crosstalk. Upon knockout of SLC38A2, a striking reversal of alanine flux in PDAC cells leading to a significant increase in alanine secretion in both basal and alanine-supplemented conditions was observed (FIG. 2a and FIG. 8a). These data supports a major role for SLC38A2 in facilitating PDAC-specific alanine influx, and it was next sought to more fully characterize impacts on intracellular alanine metabolism in PDAC cells deficient in SLC38A2. Intracellular alanine levels were significantly reduced in SLC38A2-deficient cells cultured in basal media; however, alanine levels in media supplemented with alanine increased, albeit significantly less than in control cells (FIG. 8b). To determine whether SLC38A2-deficient could sustain exchange of alanine through other transport systems, stable-isotope tracing using ¹³C₃-alanine was conducted and incorporation into TCA cycle metabolites was measured. SLC38A2 deficiency significantly decreased labeling of citrate and other TCA cycle intermediates (FIG. 8c); however, modest labeling suggesting some redundancy in alanine transport allowing exchange was still observed. It was determined that SLC38A2-dependent co-transport of sodium and alanine allows PDAC cells to concentrate intracellular alanine whereas other mechanisms could only passively equilibrate levels with extracellular concentrations. The cellular capacity to internalize and concentrate cell-permeable esterified L-alanine, which can diffuse into cells independently of transporters, was quantified. Strikingly, significantly increased efflux of de-esterified alanine in cells lacking SLC38A2, and a reduced capacity to concentrate de-esterified alanine was observed (FIG. 2b and FIG. 8d). Importantly, expression of a sgRNA-resistant SLC38A2 cDNA fully rescued intracellular alanine levels in PDAC, whereas a non-functional mutant incapable of binding sodium failed to rescue (FIG. 8e, f). Taken together, these data indicate that SLC38A2 is required and the primary driver of concentrative alanine influx in PDAC cells (FIG. 2c).

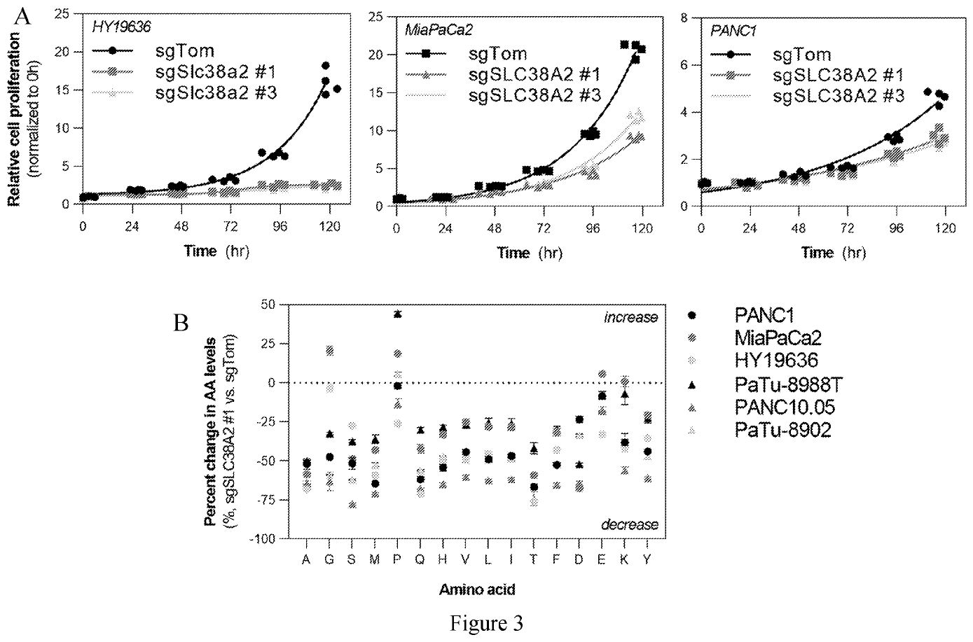

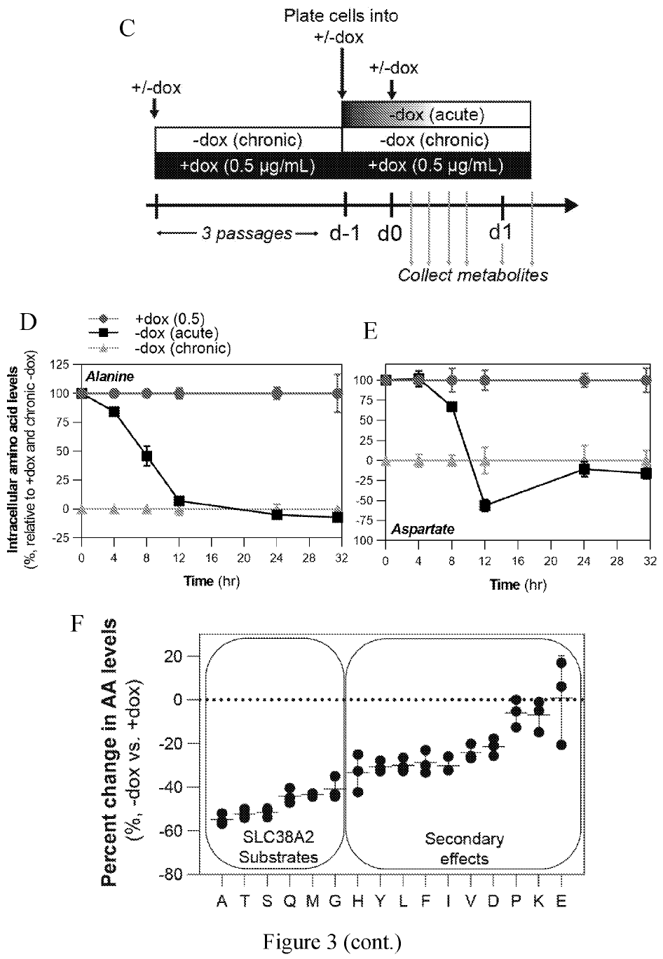

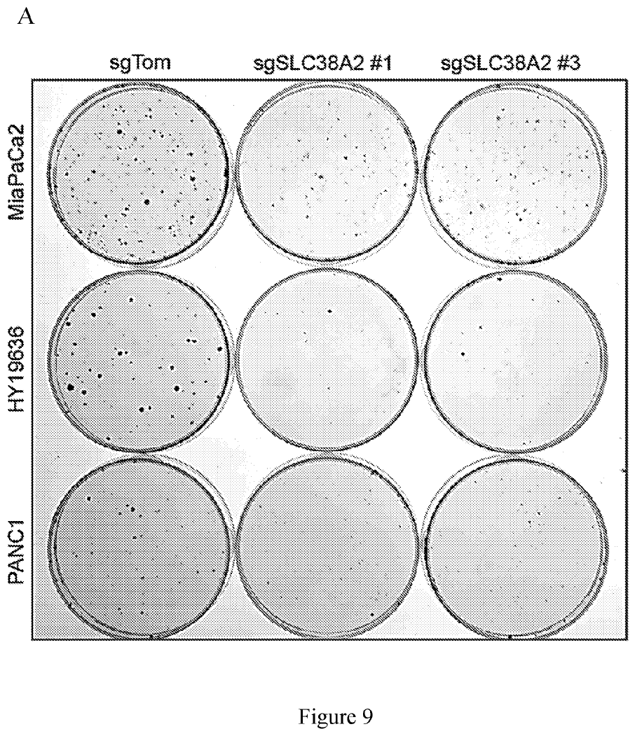

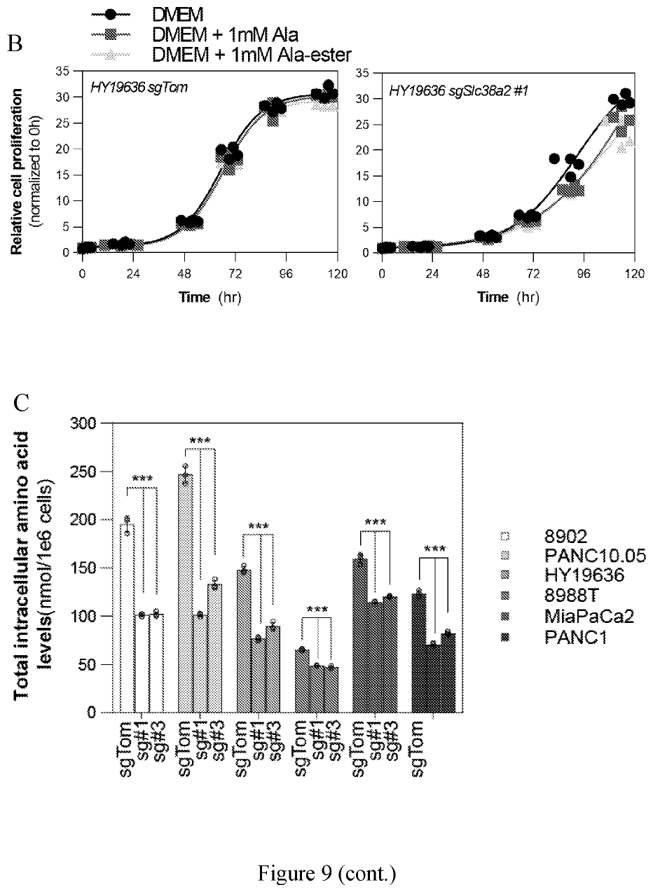

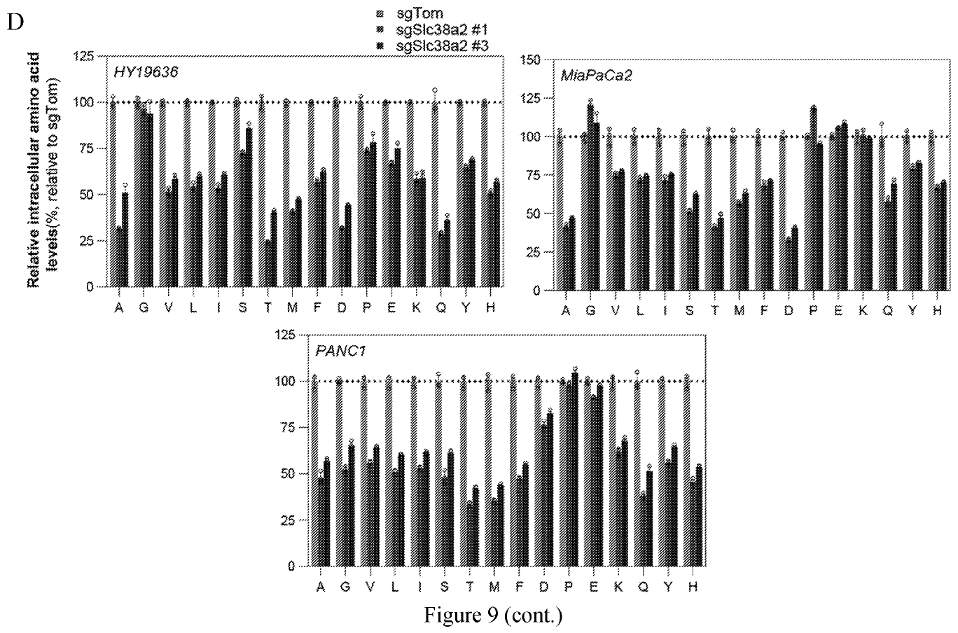

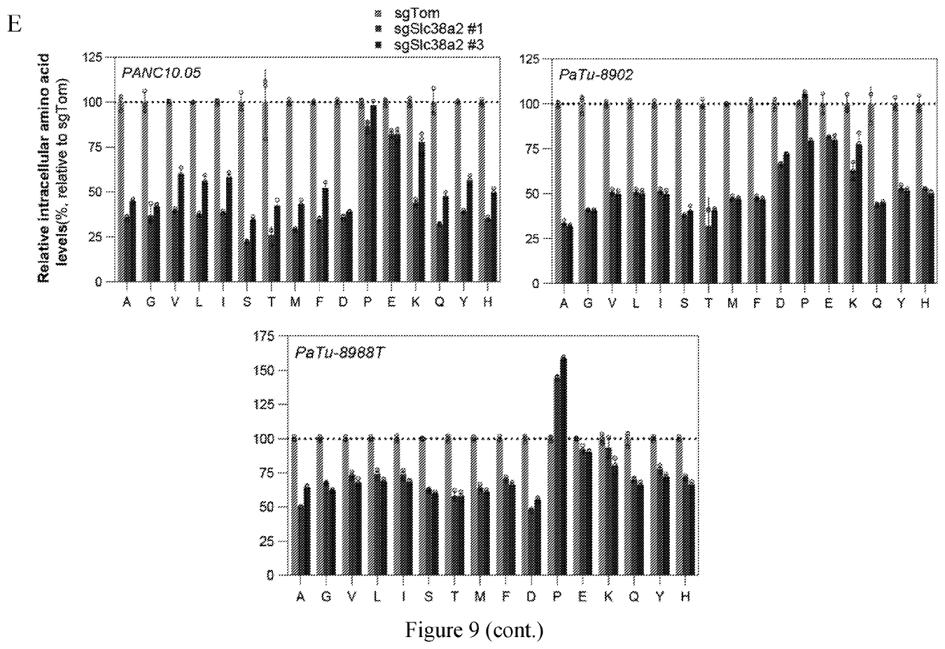

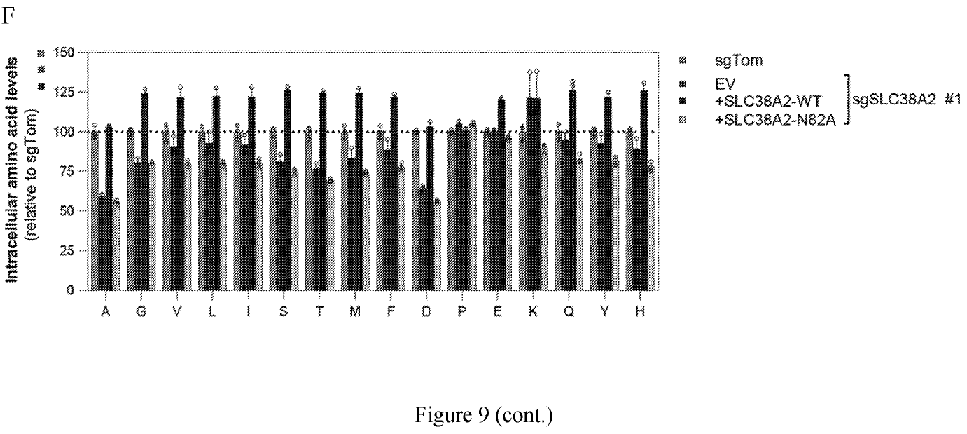

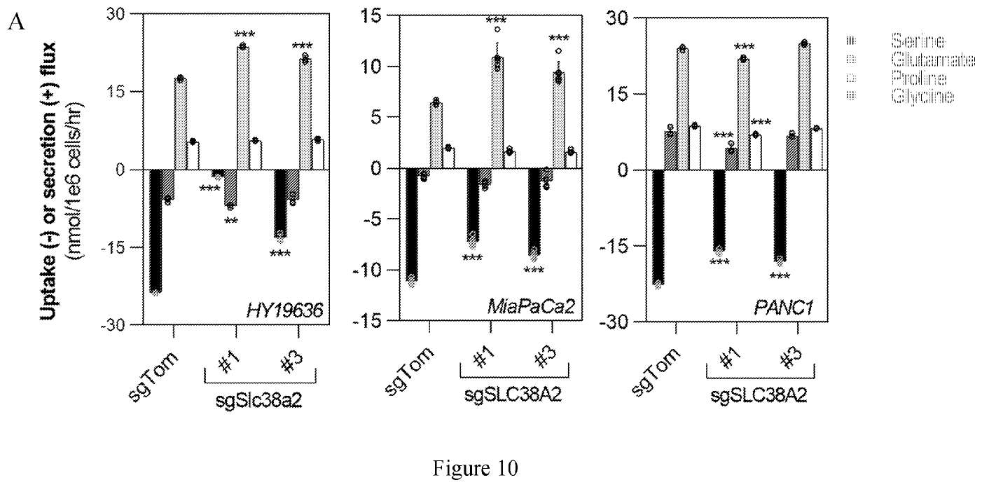

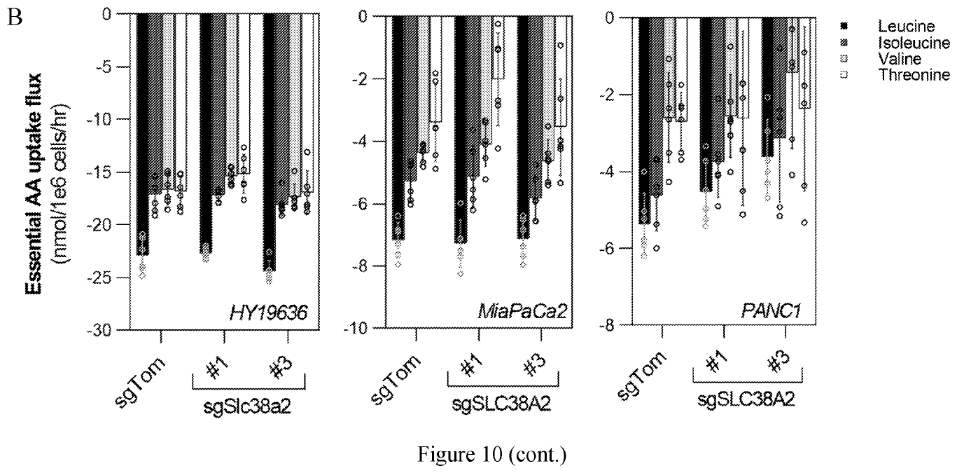

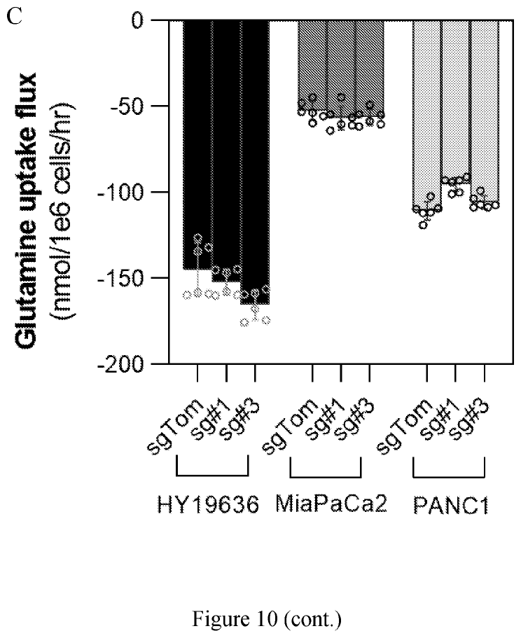

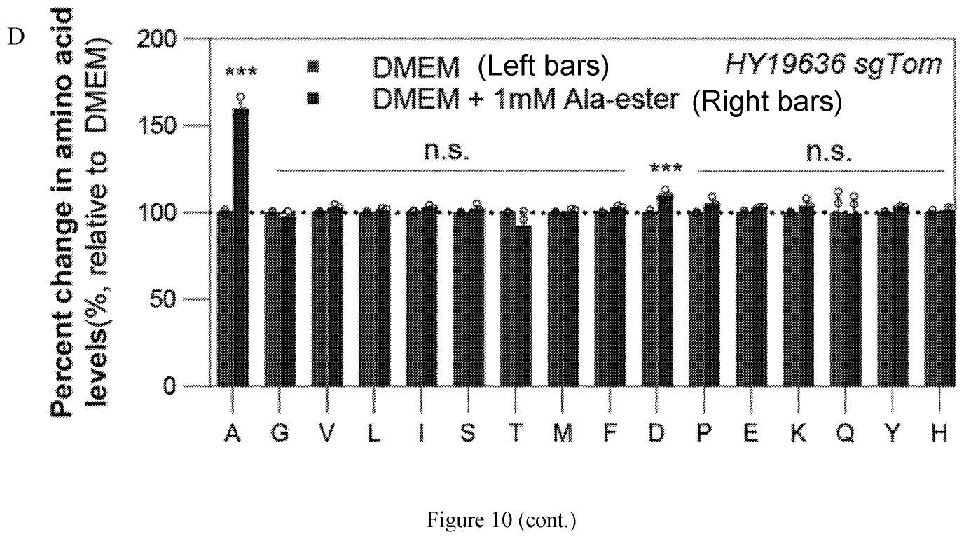

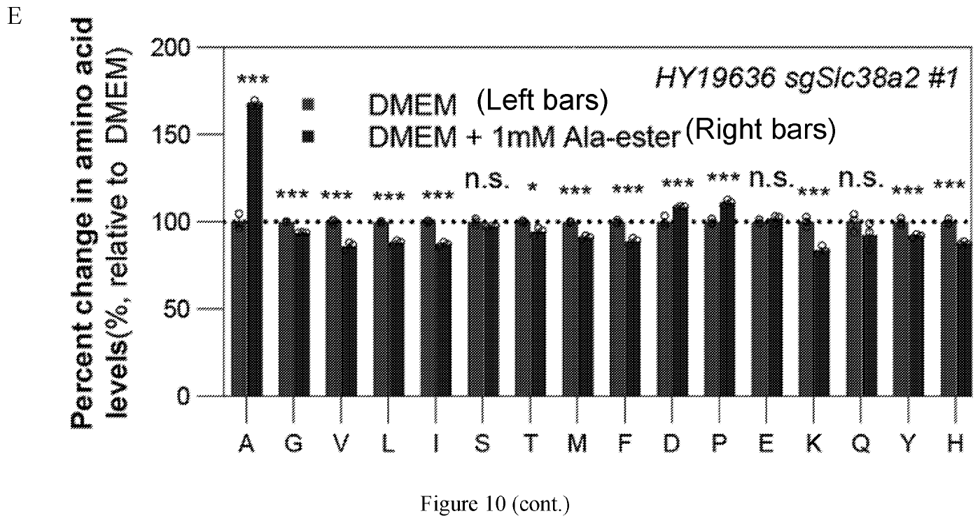

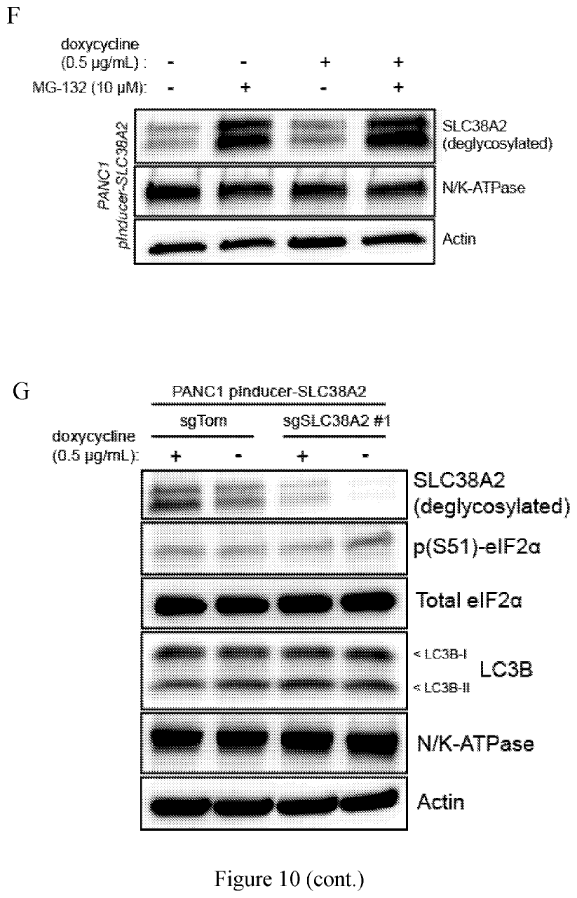

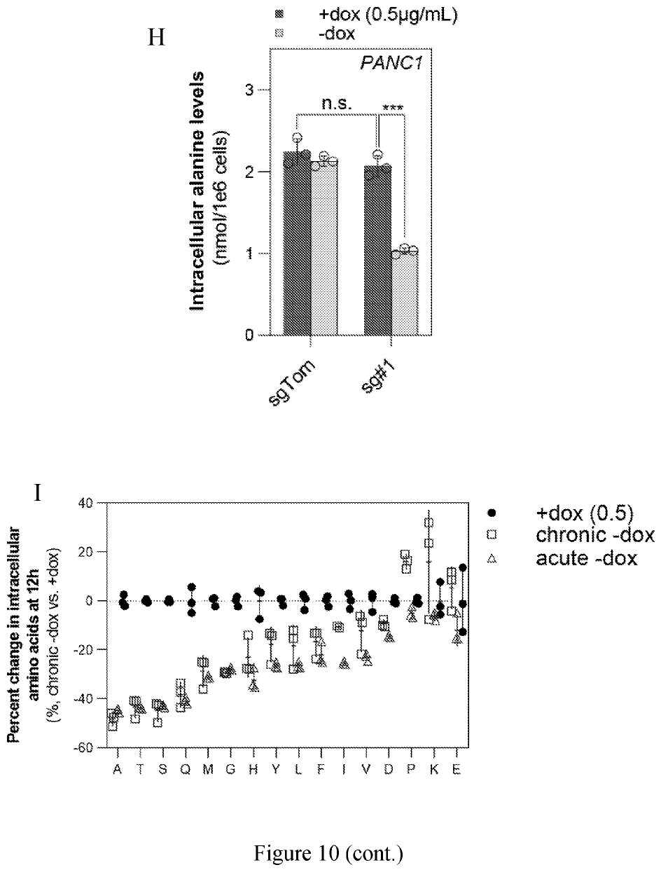

Alanine supplementation does not stimulate PDAC proliferation in replete nutrient conditions. In contrast, alanine supplementation enhanced proliferation of PDAC cells cultured in low amino acid conditions that mimic the austere PDAC microenvironment (FIG. 2d). Strikingly, loss of SLC38A2 significantly attenuated the growth-promoting effects of alanine in austere culture conditions (FIG. 2d), and resulted in significantly reduced cell proliferation and clonogenic potential even in nutrient replete conditions (FIG. 3a and FIG. 9a). However, neither exogenous alanine nor cell-permeable esterified alanine rescued the proliferation defect in SLC38A2-null cells, despite sufficiently increasing alanine levels similar to that measured in control cells (FIG. 8b, 9b). Given that alanine uptake contributes significantly to amino acid biosynthesis (FIG. 6), it was determined that defects in other amino acids may also contribute to the proliferation defect. Strikingly, a significant decrease in total amino acid levels and extensive alterations in the composition of cellular amino acids as a result of SLC38A2 across many PDAC cell lines, which was fully rescued by a sgRNA-resistant cDNA, was observed (FIG. 3b and FIG. 9c-f). Quantification of other amino acid extracellular fluxes, including SLC38A2 substrates and non-substrates, revealed significant, but relatively minor compared to impacts on alanine flux, and cell line-dependent alterations as a result of SLC38A2 loss (FIG. 10a-c). Paradoxically, SLC38A2-deficient cells exhibit broad spectrum dysregulation in amino acid levels while maintaining extracellular flux of amino acids by redundancy through other transporters. These data suggest that disruption of the capacity to influx and concentrate alanine may directly, through deficiency in alanine transport kinetics and metabolism, and/or indirectly contribute to defects in proliferation.

It was determined that the broad de-regulation of intracellular amino acid pools upon SLC38A2 loss may be due to alanine serving as an exchange factor for the maintenance of other amino acid pools (including BCAAs through SLC7A5/LAT1, as has been reported for glutamine and asparagine). In this model, the sodium gradient produced by the N/K-ATPase provides the free energy for SLC38A2 to concentrate intracellular alanine against an intracellular concentration gradient (FIG. 2c). Subsequently, concentrated cytosolic alanine provides the free energy necessary to exchange other amino acids through facilitated diffusers. Indeed, sodium binding was required for SLC38A2-dependent concentrative alanine transport (FIG. 8e, f). To determine whether alanine may serve as an exchange factor, it was investigated whether cell-permeable esterified alanine was able to rescue intracellular amino acid pools in response to SLC38A2 loss. It was determined that esterified alanine would re-establish intracellular concentrations of alanine following de-esterification in the cytosol. These data demonstrated that esterified alanine was rapidly de-esterified and secreted in SLC38A2-null cells and (FIG. 2b). If alanine serves as an exchange factor, its secretion may be coupled to exchange with other amino acids. However, esterified alanine failed to significantly impact amino acid levels in either SLC38A2 competent or deficient cell lines suggesting that alanine does not function as an exchange factor and that secretion of internalized and de-esterified alanine likely occurs through mass action-driven passive diffusion (FIGS. 10d and 10e). Furthermore, these results suggest that cells lacking SLC38A2 likely undergo metabolic rewiring and suppress proliferation in response to loss of concentrative alanine transport.

Many of the amino acids with altered intracellular levels are not reported substrates of SLC38A2 (e.g., glutamate, tyrosine, lysine, BCAAs) or are not transported by PDAC cells (e.g., aspartate) (FIG. 3b); therefore, it was hypothesized that disruption of alanine influx leads to a metabolic crisis which forces PDAC cells to rewire intracellular metabolism and suppress proliferation. To deconvolute primary and secondary metabolic effects in response to SLC38A2 loss, we ectopically expressed a dox-inducible sgRNA-resistant cDNA, which was induced and maintained during CRISPR/Cas9-mediated deletion of the endogenous locus (FIG. 3c). Notably, it was observed that minimal impacts in SLC38A2 levels in response to dox-induction in control cells and a significant accumulation of SLC38A2 in response to proteosomal inhibition, suggesting post-translational control of SLC38A2 levels in-line with previous reports, allowing us to deplete SLC38A2 acutely in a dox-dependent manner (FIG. 10f, g). In addition, chronic SLC38A2-null cells were established by culturing dox-controlled cDNA rescue cells in the absence of dox for several passages to allow for secondary metabolic rewiring to occur (FIG. 3c). Upon removal of dox, we observed a rapid decrease in SLC38A2 protein and alanine levels concordant with our chronic knockout experiments (FIG. 10h, i). Importantly, expression of dox-inducible SLC38A2 fully rescued alanine levels, and cDNA expression had no significant impact on alanine levels in control cells (FIG. 10h). Furthermore, a robust increase in eIF2a phosphorylation and no appreciable alteration in LC3B lipidation was observed, suggesting activation of specific amino acid stress responses as a direct result from acute SLC38A2 loss (FIG. 10g). To deconvolute the primary and secondary metabolic consequences of SLC38A2 loss, we conducted short-term acute cDNA-withdrawal metabolomics experiments. After dox withdrawal, alanine levels rapidly decreased to levels similar to chronic SLC38A2 loss and were sustained over the remaining time course (FIG. 3d). Rapid, delayed kinetic decreases in several other amino acids (e.g., aspartate) were also observed upon dox-withdrawal suggesting that the metabolic crisis occurs as a direct result of SLC38A2 loss (FIG. 3e). Furthermore, after 12 hours dox-withdrawal several amino acid levels decreased significantly below the level observed in chronic SLC38A2-null cells (e.g., histidine, tyrosine, aspartate) followed by an increase to chronic levels after 24 and 36 hours (FIG. 3e and FIG. 10i). These data establish that small neutral amino acids, including alanine, serine, and threonine, are transported by SLC38A2 in intact cells, and provide evidence of a secondary metabolic crisis impacting other amino acid pools (FIG. 3f). Taken together, intracellular amino acid level homeostasis is intimately connected to a complex transport network, and disruption of key components of that network has profound and durable impacts on cell metabolism.

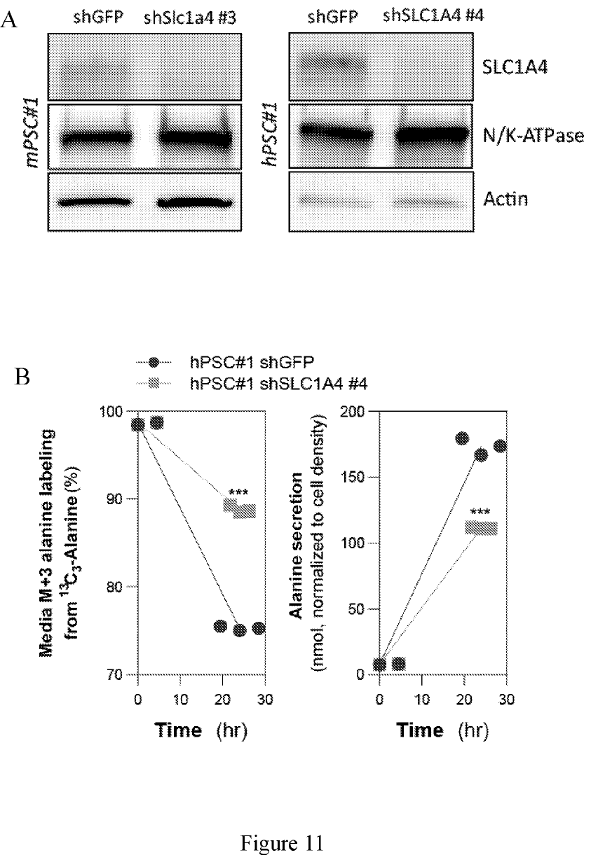

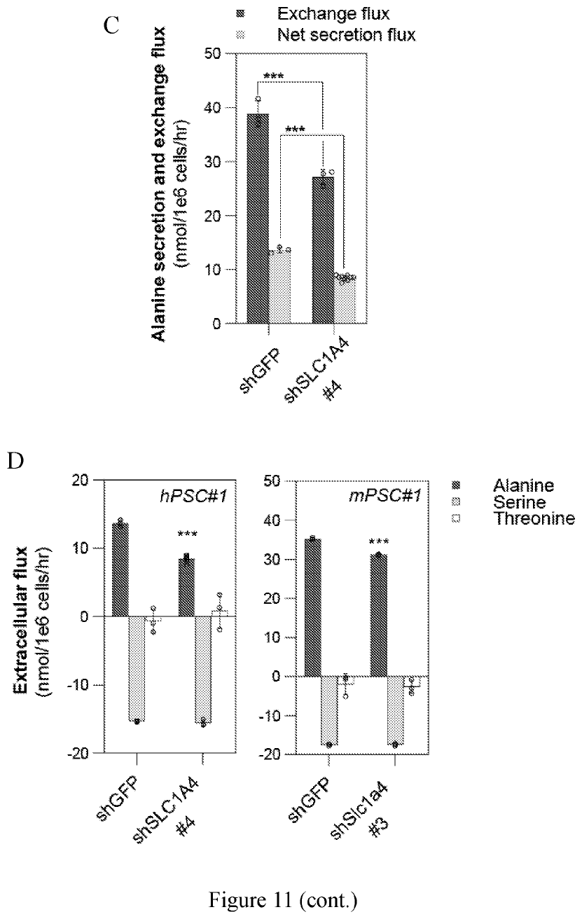

Export of alanine from stellate cells was studied. To determine if SLC1A4 may play a role in alanine export, given its specific expression in PSCs, its expression was suppressed using RNAi (FIG. 11a). Knockdown of SLC1A4 in PSCs resulted in a significant reduction in net alanine secretion flux and reduced kinetics of extracellular ¹³C₃-alanine dilution arising from exchange (FIG. 11b, c). Interestingly, there was no impact on other reported substrate fluxes, such as serine and threonine, supporting the concept that transporter specificity is influenced by the cellular context (FIG. 11d). Cysteine, although a reported substrate of SLC1A4, is rapidly oxidized to cystine in vitro, and consequently was not detectable at appreciable levels in conditioned media. Notably, a significant impact on PSC proliferation upon knockdown of SLC1A4 was not observed (FIG. 11e). The magnitude of inhibition on alanine secretion, while significant, was not complete, suggesting that SLC1A4 operates in concert with other diffusive transporters to facilitate alanine secretion by PSCs. Given that SLC38A2 is indispensable to concentrate alanine in PDAC, it was hypothesized that targeting SLC38A2 may be a strategy for blocking PSC-PDAC alanine exchange in pancreatic cancer.

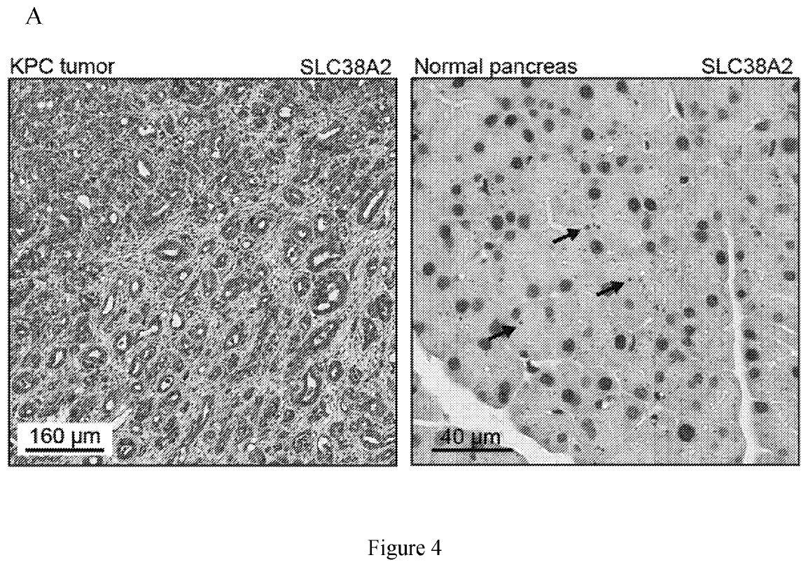

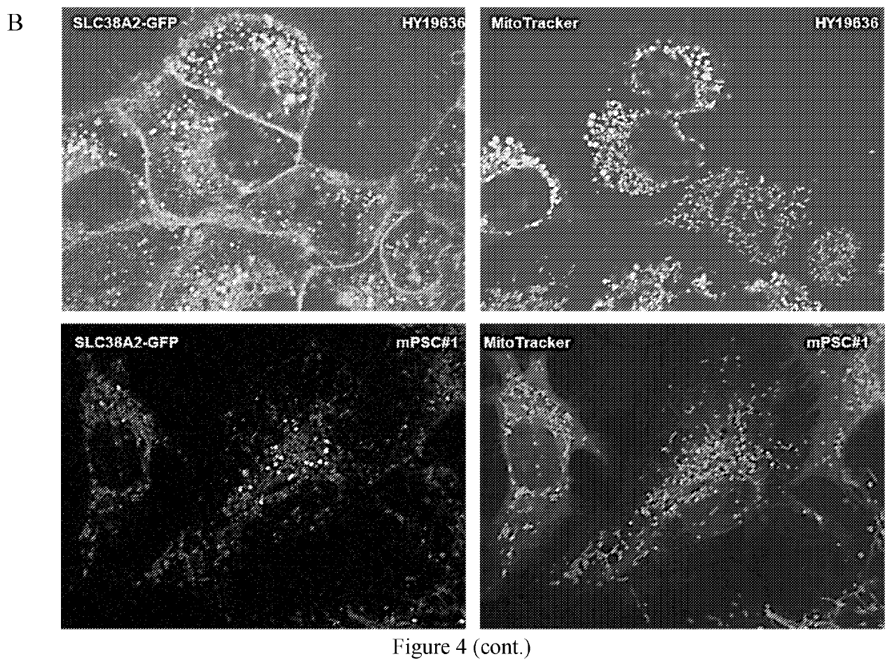

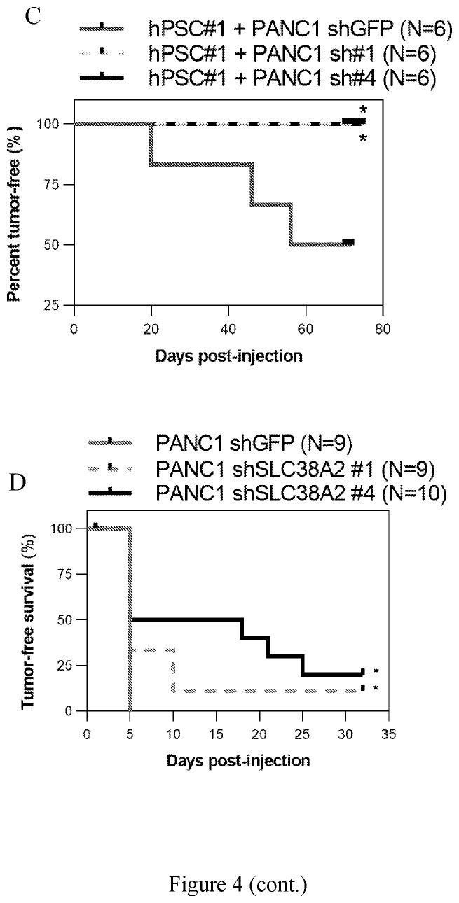

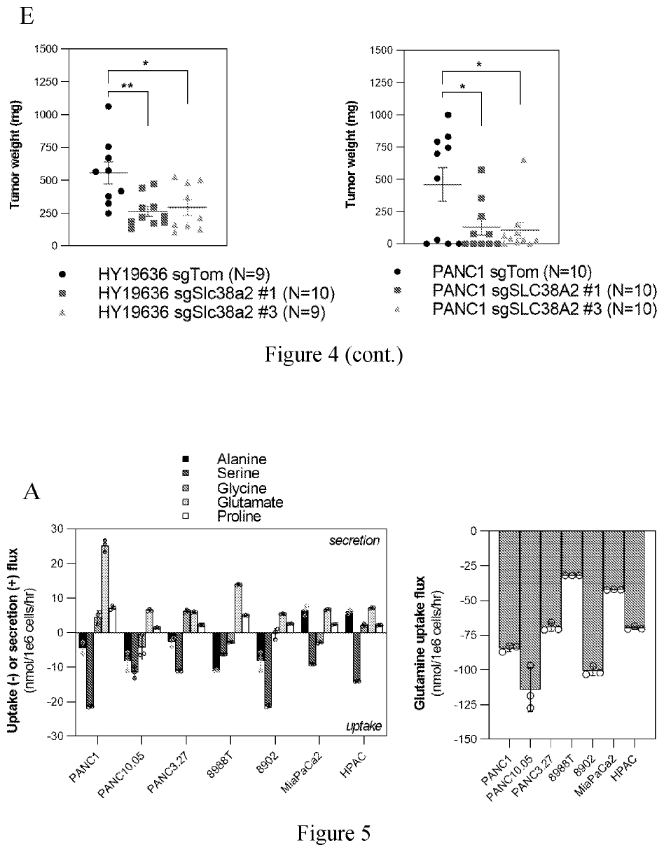

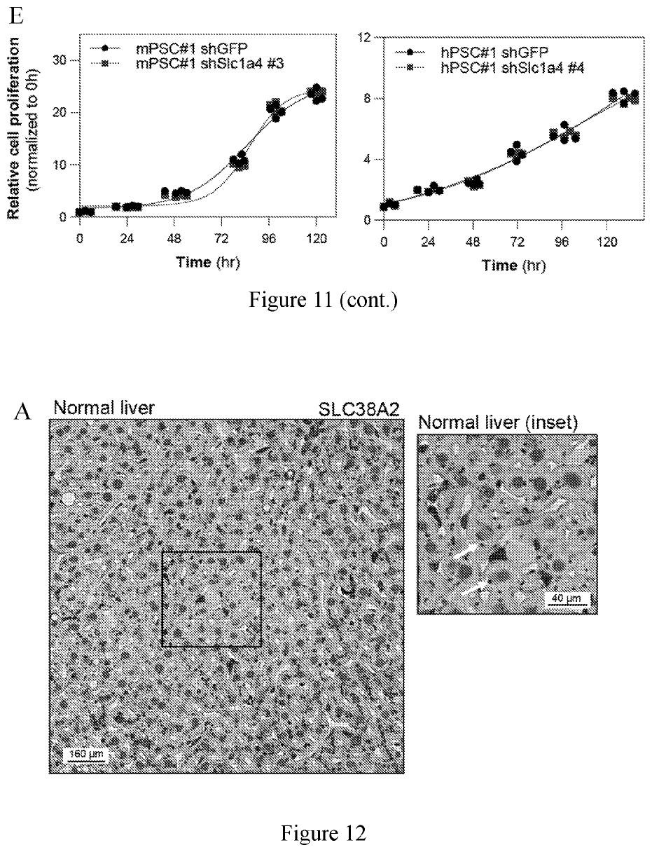

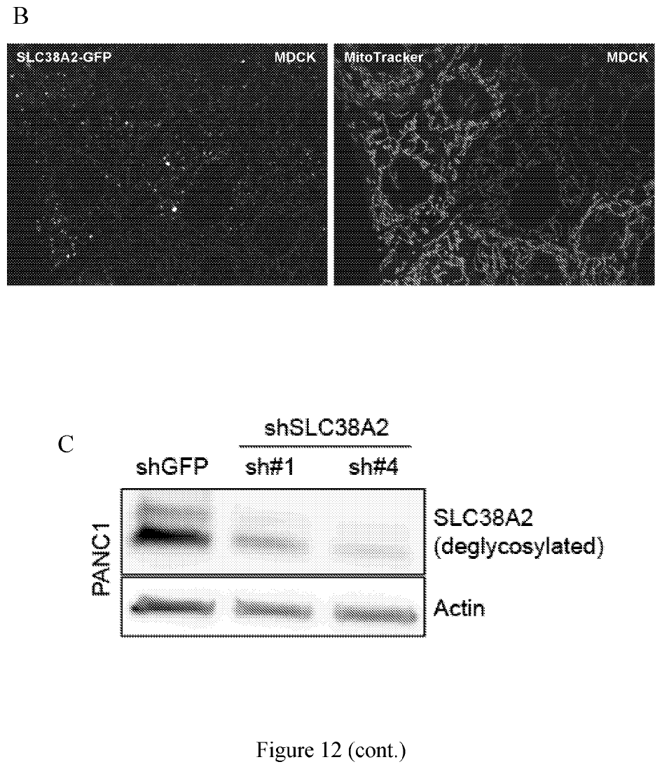

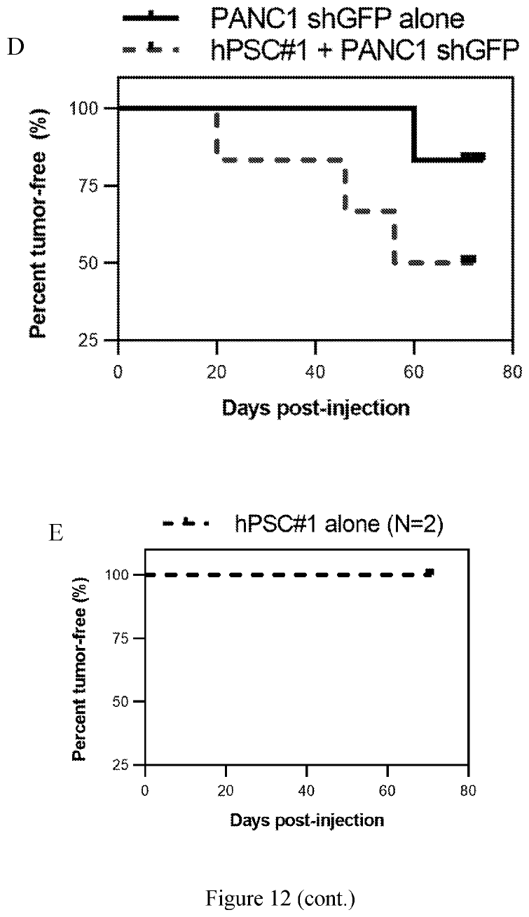

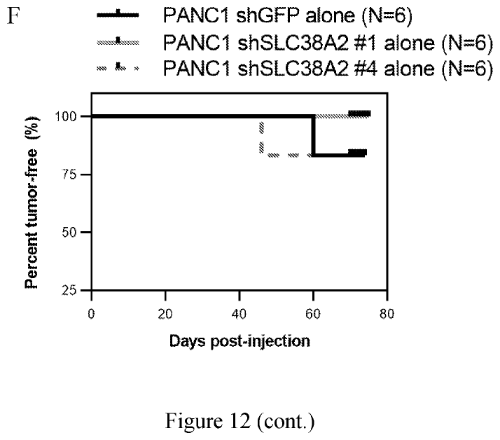

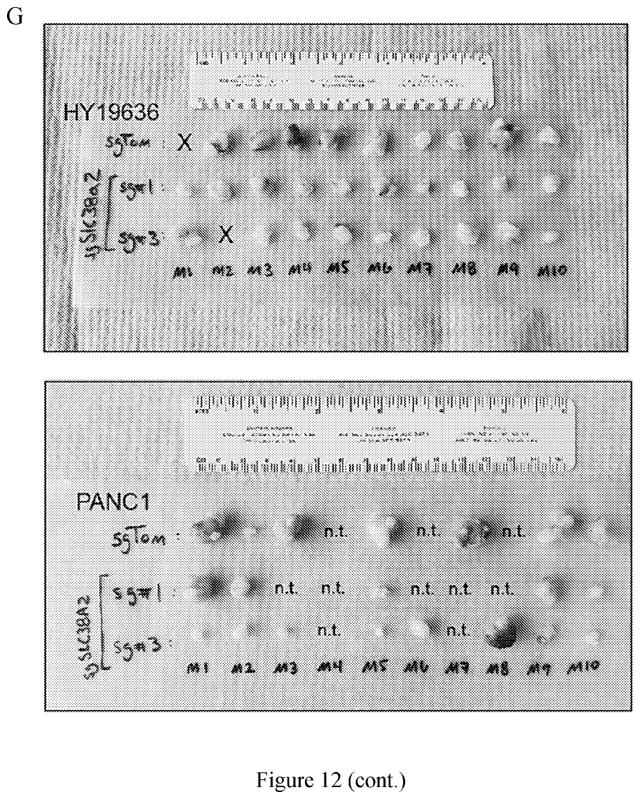

The pancreatic tumor microenvironment is thought to be extremely nutrient and oxygen austere owing to the intense fibrotic stroma, increased interstitial pressure, and leaky vasculature. Thus, nutrients within the PDAC microenvironment are likely locally supplied and shared between stromal cell populations. To determine whether PDAC-specific SLC38A2 expression is also observed in vivo, tumors derived from the well-established pancreatic cancer ‘KPC’ (LSL-Kras^{G12D}; Trp53^{lox/+}; p48Cre+) mouse model for SLC38A2 were stained. Strikingly, strong and specific staining in PDAC cells and low or no staining in the surrounding stroma was observed (FIG. 4a). As SLC38A2 is thought to be ubiquitously expressed in normal tissues, normal murine pancreas and liver was stained and it was observed punctate intracellular staining suggesting that SLC38A2 in normal tissues may be predominantly non-plasma membrane localized and inactive (FIG. 4a and FIG. 12a). Thus, PDAC cells may intrinsically activate expression and localization of SLC38A2 to facilitate PSC-PDAC alanine crosstalk during tumorigenesis. To confirm that differential localization is also observed in PDAC cell lines, SLC38A2-GFP were transiently transfected into primary PDAC and non-malignant mPSC #1 and canine kidney epithelial (MDCK) cell lines and determined localization patterns by confocal microscopy. In-line with previous results, it was observed that robust plasma membrane localization of SLC38A2-GFP in PDAC cells, whereas non-malignant cells localize SLC38A2 predominantly to intracellular endomembranes (FIG. 4b and FIG. 12b).

To determine whether targeting SLC38A2-mediated alanine uptake was sufficient to inhibit PSC-PDAC alanine crosstalk in vivo, a co-injection strategy similar to previous studies was utilized. PSC-PDAC co-injection significantly promotes tumor initiation and growth; therefore, subcutaneous xenografts injecting control or SLC38A2 knockdown PDAC cells alone or co-injected with PSCs were performed (FIG. 12c). Enhanced tumor initiation in PDAC cells co-injected with PSCs was observed (FIG. 12d). Notably, enhanced tumor initiation by PSC co-injection was completely abolished by knockdown of SLC38A2 (FIG. 4c and FIG. 12e). To further demonstrate the role of SLC38A2-mediated alanine crosstalk in tumor initiation and growth, orthotopic xenograft and allograft studies were conducted using SLC38A2 knockdown or knockout PDAC cells into a more physiologically-relevant pancreas microenvironment containing naïve fibroblasts. Consistent with the subcutaneous xenograft results, a delay in tumor initiation and tumor burden was observed in orthotopic xenograft and syngeneic allograft assays (FIG. 4d, e and FIG. 12f, g).

### Discussion

It was demonstrated herein that pancreatic cancer and stellate cells evolve a niche within the tumor microenvironment to exchange alanine through differential expression of SLC38A2 and SLC1A4. These data illustrate differential alanine transporter expression is required for the maintenance of alanine exchange within the PDAC tumor niche. Transport and utilization of glucose requires both transport and the activity of hexokinase to (1) prevent exchange through phosphorylation, and (2) decrease intracellular glucose concentrations to drive more transport. In contrast, alanine and other amino acids are not enzymatically modified for intracellular sequestration, and rather cells have evolved a complex network of transporters to promote net influx and maintain intracellular concentrations necessary for intermediary metabolism. Cancer cells hijack specific transporters in this network to enhance influx of nutrients required to fuel their metabolic demands.

Methods.

Cell Culture:

The cell lines PANC1, PANC3.27, BxPC3, HPAC, MiaPaCa2, PaTu-8988T, PANC10.05, MDCK, and PaTu-8902 were obtained from ATCC or the DSMZ. hPSC #1 and mPSC #1 were obtained as previously described. Primary murine PDAC cell lines (HY19636, MPDAC4, and HY15566) were isolated from KPC tumors using established protocols. Cells were maintained in DMEM (Corning) supplemented with 10% FBS (Atlanta Biologicals S11550H, Lot No. C18030) and 1% Pen/Strep (Gibco). Cultures were routinely verified to be negative for Mycoplasma by PCR. Cell lines were authenticated by fingerprinting, and low passage cultures were carefully maintained in a central lab cell bank.

Proliferation Assays:

Proliferation curves were obtained by plating cells at variable densities depending on growth rate into 24 well plates (HY19636 at 2-3,000 cells/well, PANC1 and MiaPaCa2 at 5,000 cells/well, and hPSC #1 and mPSC #1 at 6,000 cells/well). Cells were fixed in 10% formalin (ThermoFisher) for 10 minutes and stained with a 0.1% crystal violet solution (Sigma) containing 10% ethanol for 30 minutes. After plates were dry, dye was extracted with 10% acetic acid (Sigma) and absorbance was measured at 595 nm using a FLUOstar Omega plate reader (BMG Labtech). Absorbance measurements were background corrected and proliferation curves were determined by normalizing to day zero absorbance measurements.

Clonogenic assays were performed by seeding a single cell suspension at a density of 1,000 cells/plate into a 6 cm dish in full DMEM with 10% FBS. After 14 days (PANC1) or 7-10 days (MiaPaCa2, HY19636) cells were fixed and stained in a 6% glutaraldehyde and 0.5% crystal violet solution for 1 hour and allowed to dry prior to imaging and counting.

For low amino acid growth assays, cells were trypsinized and resuspended in austere DMEM (25 mM glucose, 10% [AA]_{DMEM}, 10% FBS) and plated directly into clear bottom 96-well plates (Corning) containing 12 technical replicates for experimental condition. Custom DMEM deficient in amino acids, glucose, sodium pyruvate, and sodium bicarbonate was obtained from US Biological (D9800) and prepared with 25 mM glucose, sodium bicarbonate, and 10% dialyzed FBS at pH 7.4. DMEM (no AA, 25 mM Glucose, 10% dialyzed FBS) was mixed with full DMEM to achieve DMEM (10% [AA]_{DMEM}, 25 mM Glucose, 10% dialyzed FBS). Wells contained 100 μL of media supplemented with 2× concentrations of alanine to reach final experimental condition of 1 mM after dilution with 100 μL of cell suspension. Cell growth after 48 hours was assessed by CellTiter-Glo 2 (Promega) and normalized to a time zero measurement made after cells were allowed to attach overnight.

Stable-Isotope Tracing, Metabolite Profiling, and Flux Analysis:

For ¹³C- and ¹⁵N-labeled alanine tracing experiments, cells were plated into 6-well plates at 250,000 cells/well and allowed to attach overnight. Cells were cultured for 24 hours in DMEM supplemented with 1 mM labeled alanine and 10% dialyzed FBS (Gibso 26400044) followed by metabolite extraction. For metabolite extraction, cells were first washed with ice cold 0.9% NaCl to remove media contamination followed by addition of 500 μL methanol (−20° C.) and 200 μL HPLC grade water (4° C.). Cells were scraped and pipeted into glass vials containing 500 μL chloroform (4° C.) and vortexed at 4° C. for 10-15 minutes. Aqueous and inorganic layers were separated by cold centrifugation for 15 minutes. 300 μL of the aqueous layer containing polar metabolites was transferred to sample vials (Agilent 5190-2243) and evaporated using a SpeedVac (Savant Thermo SPD111V). Dried samples were stored at −20° C. and re-evaporated for 5-10 minutes prior to derivatization and GC-MS analysis. Fatty acid labeling from ¹³C₃-alanine was measured by transferring 400 μL of the inorganic layer to a glass vial and evaporating under nitrogen flow in a needle evaporator prior to transesterification and GC-MS analysis. To quantify contribution to proteogenic amino acid pools, the insoluble interphase layer containing proteins was washed three times with HPLC-grade acetone and allowed to dry overnight with gentle nitrogen flow in a needle evaporator. The resulting protein pellet was hydrolyzed in 2N HCl at 95° C. for two hours with occasional vortexing and dried overnight under nitrogen flow using a needle evaporator prior to derivatization and GC-MS analysis. For amino acid and metabolite profiling, approximately 0.5-1×10⁶ cells were extracted using methanol:water:chloroform containing 2.5 nmol of uniformly (¹³Cₓ,¹⁵Nₓ)-labeled amino acids including L-alanine, L-lysine, L-hisitidine, L-arginine, L-tyrosine, L-phenylalanine, L-methionine, L-glutamic acid, L-aspartic acid, L-leucine, L-isoleucine, L-valine, L-threonine, L-proline, L-serine, and glycine; 4 nmol of ¹³C₅-labeled L-glutamine; 0.5 nmol ¹³C₃-labeled pyruvate; 5 nmol ¹³C₃-labeled lactate; 1.25 nmol ¹³C₆,¹⁵N₂-labeled cysteine, and 1 μg of norvaline. The abundance of each metabolite was quantified by the following equation:

$$AA = \frac{{x}_{standard} - \% M {0}_{X}}{100 - \% M {0}_{X}}$$

Where X_{standard} is the molar amount for each added standard (e.g., 2.5 nmol for alanine, 5 nmol for lactate) and % M0_{X} is the relative abundance of unlabeled (M+0) species ‘X’ corrected for natural isotope abundance. Multiple surrogate wells were pooled and counted to normalize metabolite abundances by cell number.

For alanine exchange experiments, PSCs were plated at 100,000 cells/well into 6-well plates and allowed to attach overnight. Media containing 1 mM ¹³C₃-alanine and 10% dialyzed FBS was then added to cells. Every 24 hours, conditioned media was collected and cold centrifuged at 1,000×g for 10 minutes to remove cell debris and flash frozen at −80° C. prior to extraction. To quantify alanine labeling and absolute abundance, 5 μL of fresh or conditioned media was extracted in 250 μL 80% methanol containing 2.5 nmol of [¹³C₃,¹⁵N] alanine internal standard followed by evaporation and GC-MS analysis.

To quantify extracellular uptake and secretion fluxes, the molar change in extracellular metabolites was determined by calculated the delta between condition and fresh media and normalizing to the cell density over the time course of the experiment using the following equation:

$${flux}_{AA} = \frac{{AA}_{f} - {AA}_{o}}{{\int }_{0}^{f} {X}_{o} {e}^{kt}}$$

where AA_{f} and AAₒ are the final and initial molar amounts of each amino acid, respectively, quantified using labeled internal standards, Xo is the initial cell density, k is the exponential growth rate (hr⁻¹), and t is the media conditioning time. To quantify alanine exchange flux, the percent dilution of exogenous ¹³C₃-alanine was multiplied by the molar quantity of alanine in the media over the conditioning period normalized to cell density over time.

Polar and Non-Polar Derivatization and GC-MS Analysis:

Polar metabolites were derivatized to form methoxime ester tert-butyl dimethyl silyl derivatives and lipids were transesterified to form fatty acid methyl esters as previously described. Derivatized samples were analyzed by GC-MS using a DB-35MS or DB-5MS column (30 m×0.25 mm i.d.×0.25 μm) installed in an Agilent 5977B gas chromatograph (GC) interfaced with an Agilent 5977B mass spectrometer (MS). The GC temperature was held at 100° C. after injection, ramped to 255° C. at 7.5° C./min, ramped to 320° C. at 15° C./min, held at 320° C. for 3 minutes, and post-run held at 320° C. for 2 addition minutes. The MS detector was operated in scan mode over a range of 100-650 m/z. Mass isotopomer distributions were determined by integrating metabolite ion fragments and corrected for natural isotope abundance using in-house algorithms.

Chemicals: U-¹³C₃-labeled alanine (CLM-2184-H), ¹⁵N-labeled alanine (NLM-454), U-¹³C₅-labeled glutamine (CLM-1822-H), U-¹³C₃-labeled lactate (CLM-1579), U-¹³C₃-labeled pyruvate (CLM-2440), and U-¹³Cₓ,¹⁵Nₓ-labeled amino acid standard mix (MSK-A2) were acquired from Cambridge Isotope Laboratories. D-glucose (Sigma), L-alanine (Sigma), L-alanine tert-butyl ester (Alfa Aesar), methoxyamine hydrochloride (Sigma), and MTBSTA+1% TBDMSCl (Sigma).

Antibodies and Western Blot:

Proteins were extracted using RIPA buffer containing fresh protease (Roche 11697498) and phosphatase (Roche 4906837) inhibitor cocktails on ice for 30 minutes. Where indicated, lysates were deglycosylated using PNGaseF (New England Biolabs P0704L) according to modified manufacturer protocol with all steps conducted at 37° C. Lysates were not boiled prior to separation on SDS-PAGE gels as heating above 50° C. caused complete loss of signal for SLC38A2. Membranes were blocked in either 5% nonfat milk or bovine serum albumin dissolved in TBS-t according to antibody manufacturer recommendations. Primary antibodies were incubated overnight at 4° C. with gentle agitation using the following antibodies and dilutions: anti-SLC38A2 (1:500; MBL, BMP081), anti-SLC1A4 (1:1000; Cell Signaling Technologies, 8442), anti-SLC1A5 (1:1000; Cell Signaling Technologies, 8057S), anti-N/K-ATPase (1:5000; Abcam, ab76020), anti-Actin (1:10,000; Sigma, A4700), anti-pS51-eIF2α (1:1000; Cell Signaling Technologies, 3398S), anti-total eIF2α (1:1000; Cell Signaling Technologies, 2103S), and anti-LC3B (1:1000; Novus, NB100-2220). After TBS-t washing, membranes were incubated with peroxidase-conjugated secondary antibodies—anti-rabbit (1:1000-1:5000; Cell Signaling Technologies, 7045S) or anti-mouse (1:2500-10,000; Cell Signaling Technologies, 7076S)—for 1 hour at room temperature and imaged by chemiluminescence (Bio-rad 1705061) using a ChemiDoc (Bio-Rad).

Lentiviral shRNA and sgRNA Targets:

All shRNA vectors were obtained from the Sigma MISSION TRC shRNA library in glycerol stock form. The target sequence and TRC number for each shRNA are as follows: shGFP: GCAAGCTGACCCTGAAGTTCAT (SEQ ID NO:1); shSLC38A2 #1: GGAGAAGATACTGTGGCAA (SEQ ID NO:2) (TRCN0000020243); shSLC38A2 #4: GAATACCAAGAGTTGTTTCTA (SEQ ID NO:3) (TRCN0000020241), shSLC1A4 #4: TGTACACCAGGGATCTGTTTG (SEQ ID NO:4) (TRCN0000418203), shSlc1a4 #3: CTTCACCAATTTGCTCGTCAT (SEQ ID NO:5) (TRCN0000326081). These shRNAs were selected after screening 5-7 shRNAs by qPCR and western blot. All sgRNAs were designed using the Broad sgRNA Designer (Broad Institute), cloned into pLentiCRISPRv2 (Addgene, plasmid #52961), and sequence verified prior to transfection. The target sequences are as follows: sgTom: GCCACGAGTTCGAGATCGA (SEQ ID NO:6), sgSLC38A2 #1: TAATCTGAGCAATGCGATTG (SEQ ID NO:7), sgSLC38A2 #3: TCTTATGCCATGGCTAATAC (SEQ ID NO:8). Lentivirus were produced by transfecting 293T cells with pLKO or pLentiCRISPRv2 constructs, pMD2.G (Addgene, plasmid #12259), and psPAX2 (Addgene, plasmid #12260) using standard Lipofectamine 3000 (ThermoFisher) protocol. All experiments using shRNA and sgRNA were conducted using pools of cells after selection to limit bias from clonal selection.

Immunohistochemistry:

Tumors resected from mice were fixed in five volumes of formalin at 4° C. for two days with gentle agitation and two formalin changes. Tumors were washed overnight with five volumes of 70% ethanol at 4° C. with gentle agitation and two ethanol changes before imaging, processing, embedding, and sectioning. Immunohistochemistry was performed on 5 μm sections. Tissues were deparaffinized and rehydrated, and antigen retrieval was performed in a steamer for 20 minutes in 10 mM pH 6.0 citrate buffer. Slides were incubated in 3% hydrogen peroxide and 50% methanol for 30 minutes and blocked in 5% goat serum and 1% bovine serum albumin in TBS-t for 30 minutes at room temperature. Primary antibody (anti-SLC38A2 1:1000; MBL BMP081) diluted in blocking buffer was added to the sections and incubated overnight at 4° C. Sections were washed with TBS-t and incubated with biotin-conjugated secondary antibody (anti-rabbit 1:200; Vector Labs BA-1000) for 30 minutes at room temperature and Acidin-Biotin Complexes (Vector Labs PK-4000) for 30 minutes. Slides were developed by 3,3-diaminobenzidine (Vector Labs SK-4100) for 3-5 minutes and quenched with water followed by hematoxylin staining for 3-5 minutes. Sections were dehydrated and mounted in permount mounting medium (Fisher Scientific SP15-100) and allowed to dry overnight before slide imaging.

Proteomics Data Analysis:

Quantitative proteomics data were generously supplied by Joseph D. Mancias. Principal Component Analysis (PCA) and differential expression analysis were conducted on log₂ fold changes using Matlab (MathWorks). Metabolism proteins were queried from a published list of approximately 3,000 proteins. Analysis of transporter proteins included all gene products with ‘SLC’ in the gene symbol and excluded ABC transporters.

Microscopy Imaging:

Live cell imaging of SLC38A2-GFP was performed on cells transiently transfected using lipofectamine 3000 in 35 mm plates that incorporate a No. 1.5 cover-slip-covered well (Mattek Corp) with an inverted Zeiss 800 laser scanning confocal microscope (Oberkochen). MitoTracker™ Red CMXRos was used to stain mitochondria. Cells were incubated in media containing MitoTracker for 15 minutes, after which the media was aspirated and replaces with fresh media.

In Vivo Xenograft and Allograft Experiments and Ultrasound Tumor Monitoring:

For subcutaneous co-injection tumor initiation studies, PANC1 cells were infected with lentiviral shRNAs targeting SLC38A2 or GFP as a control shRNA and selected with puromycin (2 μg/mL) for three days. 200,000 PANC1 shGFP or shSLC38A2 cells were resuspended in 100 μL HBSS with or without 1×10⁶ hPSC #1 cells and subcutaneously injected into bilateral lower flanks of 7-8 week old NCr nude mice (Taconic). A similar protocol was followed for co-injection tumor initiation studies using hPSC #1 cells injected with lentiviral shRNAs targeting SLC1A4 or GFP co-injected with parental PANC1 cells. Tumor initiation was monitored two to three times per week by caliper measurement. Tumor initiation was considered if length and width were measured to be ≥1 mm each.

For orthotopic xenograft experiments, PANC1 cells were infected with lentiviral shRNAs or sgRNAs targeting SLC38A2 or GFP/Tomato as a control and selected with puromycin (2 μg/mL) for three days. For syngeneic orthotopic allograft studies, HY19636 cells were infected with lentiviral sgRNAs targeting SLC38A2 or Tomato and selected with puromycin (2 μg/mL) for three days. 500,000 PANC1 cells or 10,000 HY19636 cells were resuspended in 10 μl HBSS and 10 μl growth factor-reduced matrigel (Corning 356231) per injection and kept on ice. Female 7-8 week old NCr nude or C57BL/6J mice were used for xenograft and allograft experiments, respectively. An incision was made near the spleen which was gently removed from the peritoneal space to expose the pancreas. The 20 μL cell:matrigel suspension was slowly injected into the tail of the exposed pancreas using either a Hamilton or insulin syringe (BD 324702). After injection, the needle was held in place by tweezers briefly to allow the matrigel to polymerize before gently removing the needle and re-introducing the spleen and pancreas into the animal. The peritoneum was sutured and the wound was closed with surgical staples. Buprenorphine was administered by intraperitoneal injection and immediately after surgery and every 12 hours for 48 hours.

After injection, all mice were allowed to recover from surgery for five days before screening for tumor initiation by non-invasive 3-D ultrasound imaging (VisualSonics Vevo 770) twice a week under anesthesia using 1-3% isoflurane via nose cone. Tumor initiation was considered if volume ≥1 mm³ and sustained or increased in volume over the course of the experiment. Euthanasia and tumor resection was performed at the conclusion of the experiment and tumors were confirmed by histology.

All mouse experiments with human and mouse PDAC cell lines were approved by the NYU Institutional Animal Care and Use Committee (IACUC) under protocol numbers IA16-00507 and IA16-01331.

Molecular Cloning:

To clone sgRNA-resistant SLC38A2-WT and SLC38A2-N82A cDNA, codons recognized by the target sequence were silently mutated to prevent sgRNA recognition by fragment PCR of SLC38A2 cDNA (Dharmacon MGC, clone ID 3874551). Fragments containing sgRNA-resistant sequence and AAC→GCT mutation (N82A) were isolated by PCR and assembled by Gibson assembly (New England Biolabs E2621L). The assembled fragments were PCR amplified and recombined into pDONR221 (Invitrogen 12536017) using Gateway assembly (Invitrogen 11789100). Inserts were recombined into either pLentiCMVBlast (Addgene, plasmid #17451) or pInducer20 (Addgene, plasmid #44012) using Gateway assembly (Invitrogen 11791100). To clone SLC38A2-GFP, SLC38A2 cDNA was amplified from pDONR221-SLC38A2-WT by PCR to include restriction sites for XhoI (5′) and EcoRI (3′). The PCR product for SLC38A2 and the pEGFP-N1 vector were digested with XhoI and EcoRI and ligated into pEGFP-N1 (Clonetech) using the Rapid DNA Dephos and Ligation Kit (Roche 4898117). Colonies were sequenced to confirm correct insert sequence and, if necessary, if tetO repeats were fully intact.

Statistical Analysis:

Statistical analysis was performed using Prism (GraphPad).

### Example 2

The following example provides a description of the methods of the present disclosure.

Most of the prior art focus of amino acid needs in cancer have been on glutamine.

Identification of SLC38A2 (aka SNAT2) in current disclosure was done via complex proteomics to look at patterns of thousands of proteins, identifying 1380 proteins of interest which identified several transporters, 3 upregulated on the cancer end of which 1 was SLC38A2.

α-(Methylamino) isobutyric acid (MeAIB) is a competitive inhibitor of the neutral amino acid transport A system which includes many transporters. MeAIB has been used to inhibit amino acid transport activity in A transporters (including SLC38A2/SNAT2).

Fluvoxamine is an anti-depressant, used herein, which acts as a selective serotonin reuptake inhibitor. Anti-cancer activity by disrupting actin in glioma has been previously reported, activation of caspase pathway in liver cancer cell lines, and in leukemia/lymphoma via unknown mechanisms. However, SSRIs inhibiting alanine transport in cancer or inhibiting SLC38A2 is unknown.

Homology Modeling Using I-TASSER.

The primary amino acid sequence for isoform 1 of SLC38A2 (RefSeq NP_061849.2) were acquired from NCBI and modeled using I-TASSER using the bacterial leucine transporter LeuTₐₐ from Aquifex aeolicus (PDB: 3TT1) as a template. The following validated structural constraints were also included: disulfide contact between Cys245 and Cys281 (UniProt) and 3 Å distance between residues Asn82 and Thr384 representing sodium interaction.

Drug Docking.

The putative alanine binding pocket was identified by modeling the solvent accessible surface in PyMol using solvent radius of 1.4 Å. FDA approved drugs were screened as noncovalent ligands for SLC38A2 by molecular docking using AutoDock Vina. Docking was performed within a 15 Å×15 Å×15 Å located 2 Å from the sodium atom in the predicted solvent accessible cavity.

In Vitro Hit Validation.

Fluvoxamine (FVX), fluoxetine (FLX), paroxetine (PXT), sertindole (SND), and blonanserin (BNS) were acquired from Cayman Chemicals. Desipramine (DPM), α-(Methylamino) isobutyric acid (MeAIB), and L-alanine were acquired from Sigma. Stocks were prepared at 10 mM or 7.5 mM (BNS) in DMSO. Cells were treated with vehicle (DMSO) or varying concentrations of drug for indicated time. Conditioned media was collected and centrifuged at 1,000×g to remove cell debris followed by extraction of metabolites. For metabolite extraction, cells were first washed with ice cold 0.9% NaCl to remove media contamination followed by addition of 500 μL methanol (−20° C.) and 200 μL HPLC grade water (4° C.). Cells were scraped and pipeted into glass vials containing 500 μL chloroform (4° C.) and vortexed at 4° C. for 10-15 minutes. Aqueous and inorganic layers were separated by cold centrifugation for 15 minutes. 300 μL of the aqueous layer containing polar metabolites was transferred to sample vials (Agilent 5190-2243) and evaporated using a SpeedVac (Savant Thermo SPD111V). Dried samples were stored at −20° C. and re-evaporated for 5-10 minutes prior to derivatization and GC-MS analysis. For amino acid and metabolite profiling, approximately 0.5-1×10⁶ cells were extracted using methanol:water:chloroform containing 2.5 nmol of uniformly (¹³Cₓ,¹⁵Nₓ)-labeled amino acids including L-alanine, L-lysine, L-hisitidine, L-arginine, L-tyrosine, L-phenylalanine, L-methionine, L-glutamic acid, L-aspartic acid, L-leucine, L-isoleucine, L-valine, L-threonine, L-proline, L-serine, and glycine; 4 nmol of ¹³C₅-labeled L-glutamine; 0.5 nmol ¹³C₃-labeled pyruvate; 5 nmol ¹³C₃-labeled lactate; 1.25 nmol ¹³C₆,¹⁵N₂-labeled cystine, and 1 μg of norvaline.

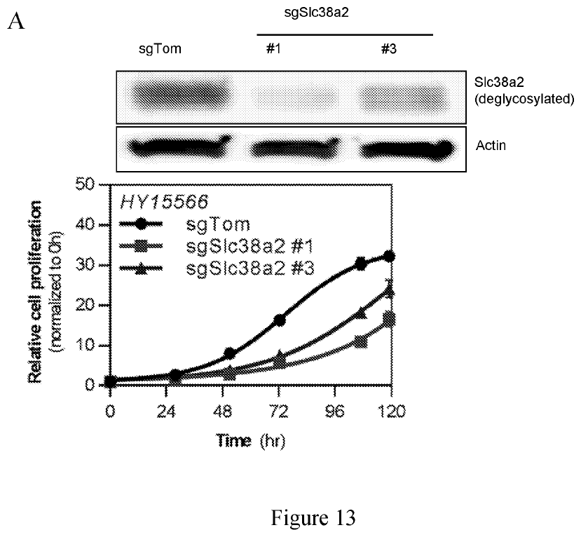

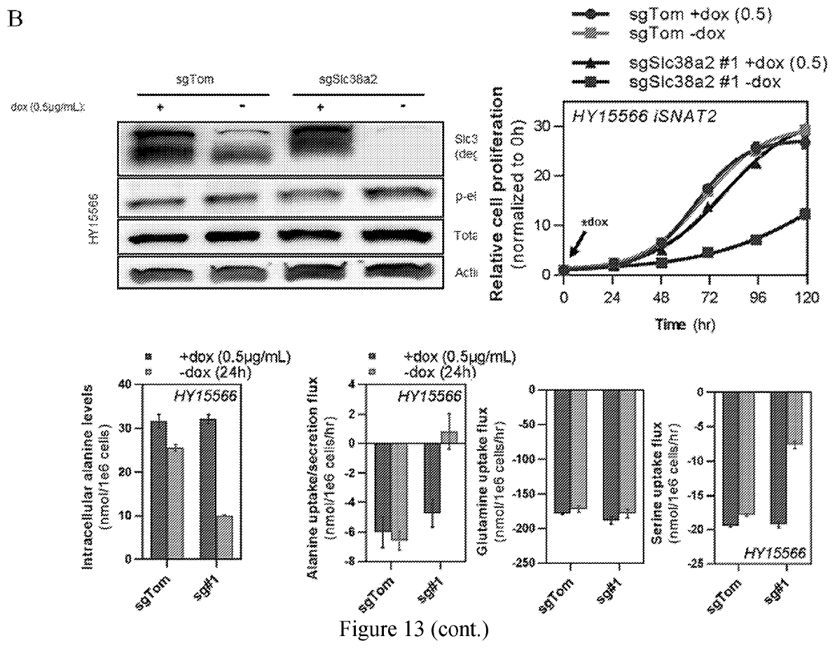

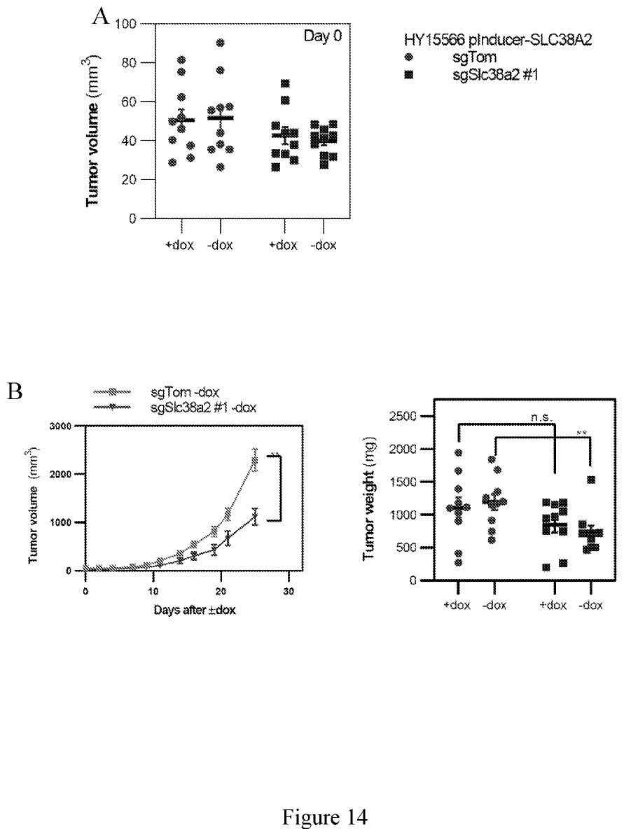

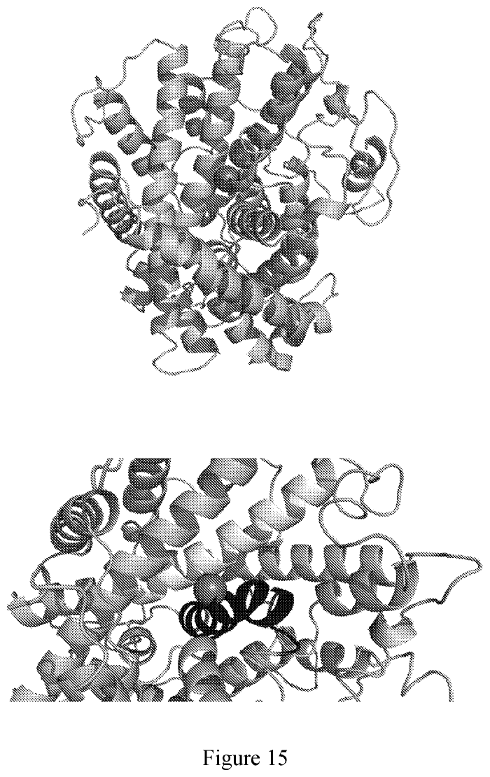

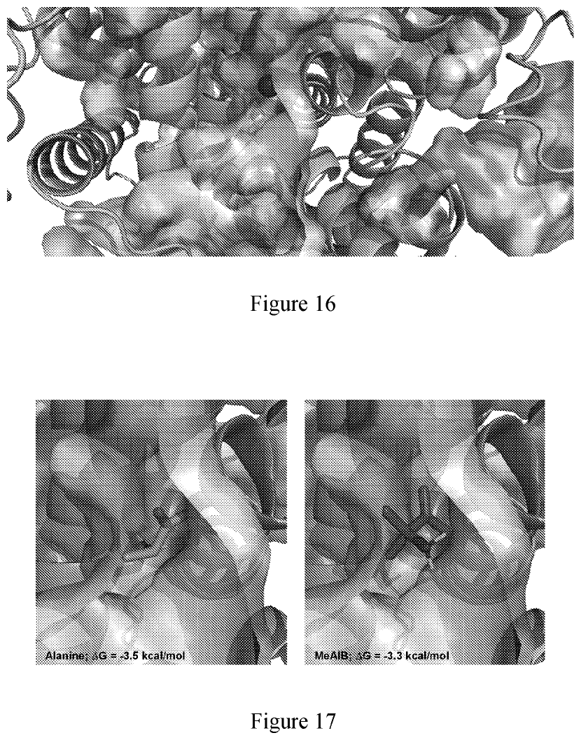

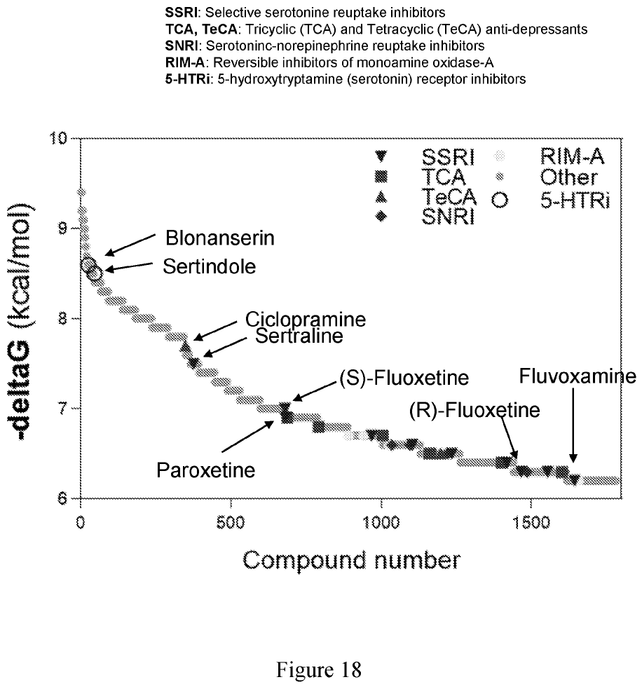

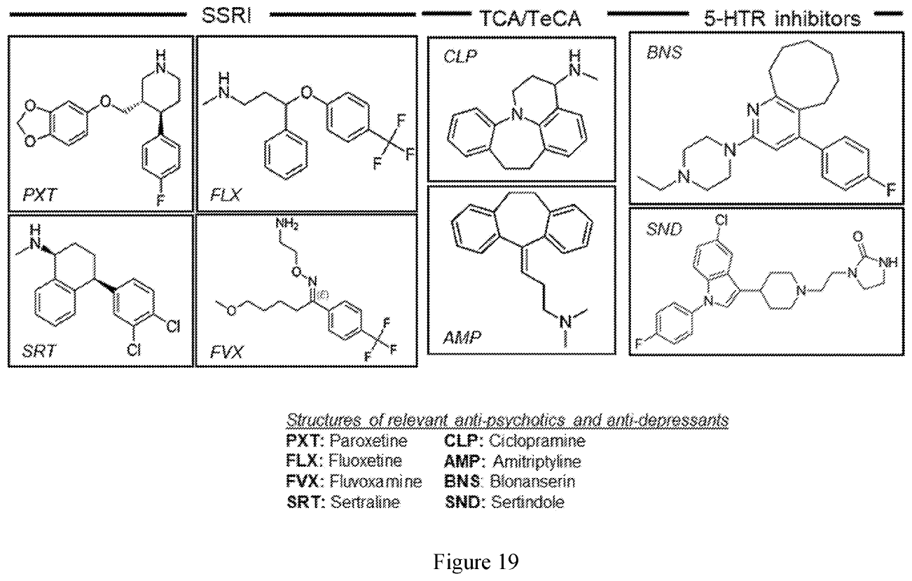

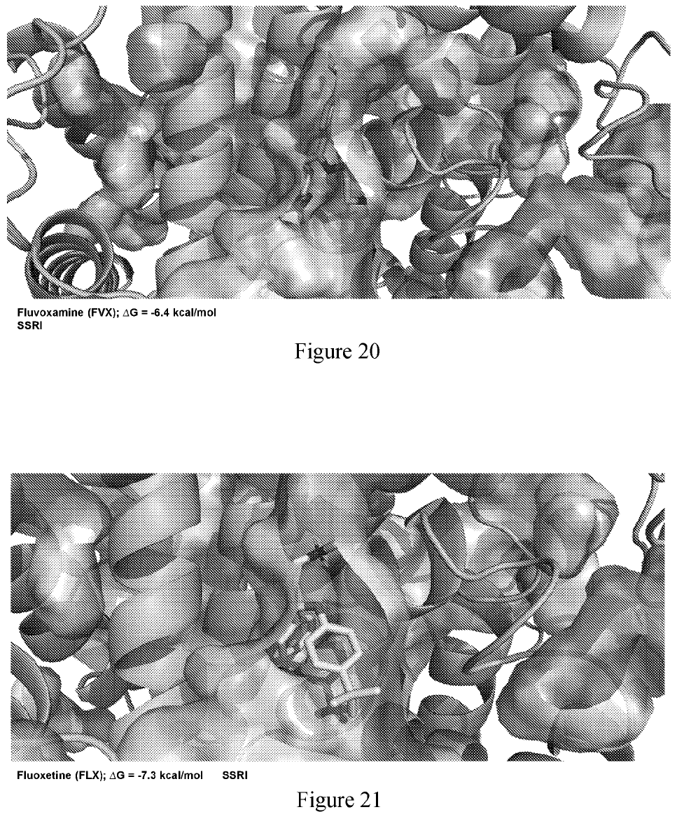

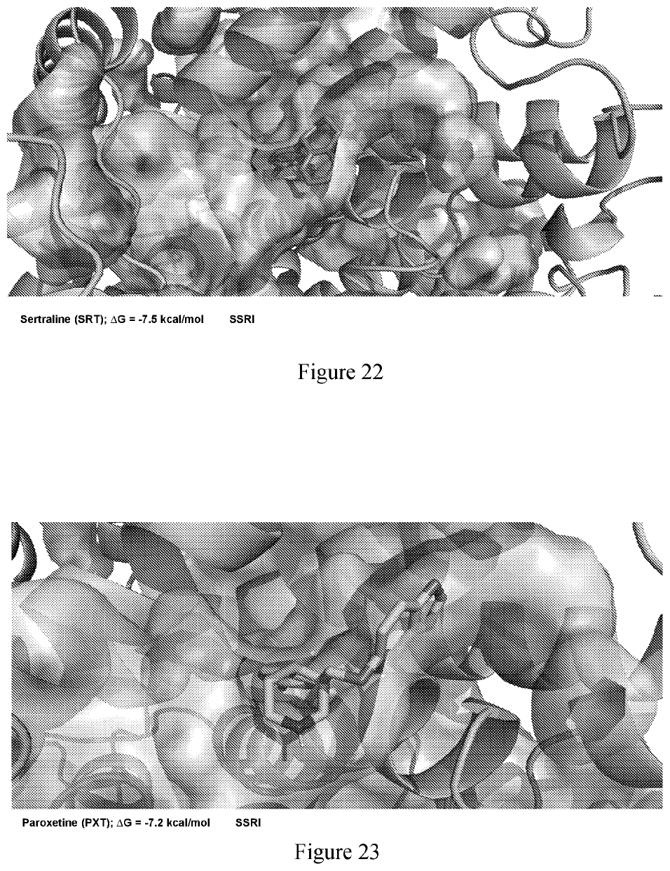

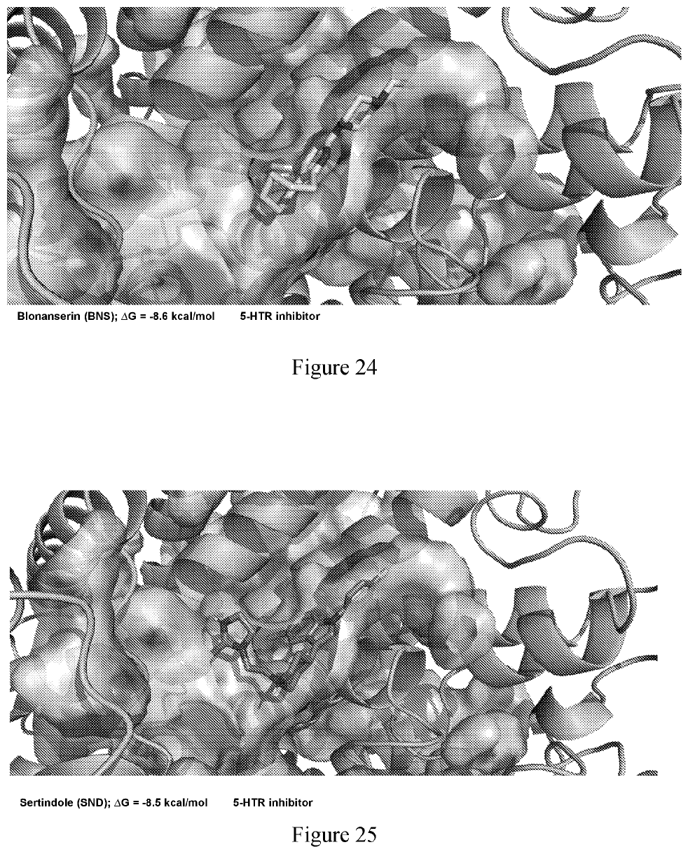

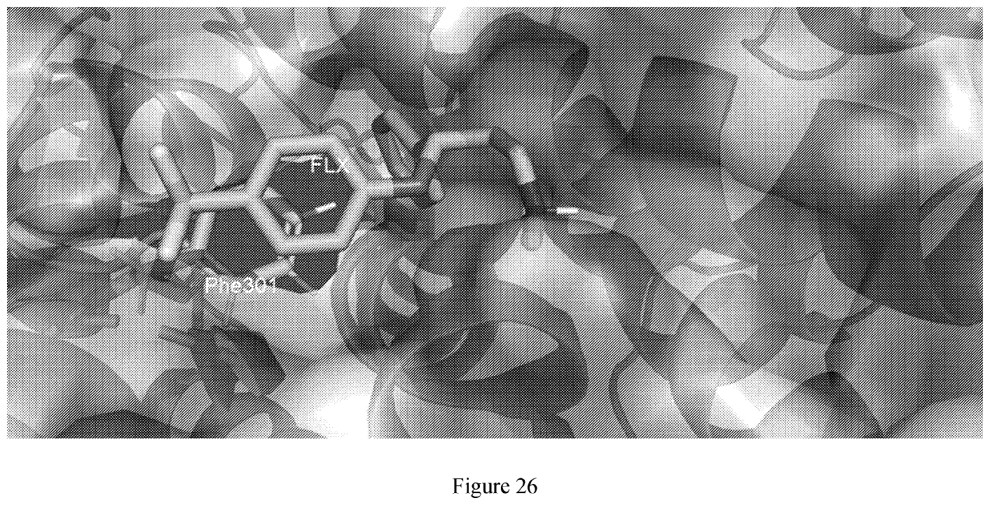

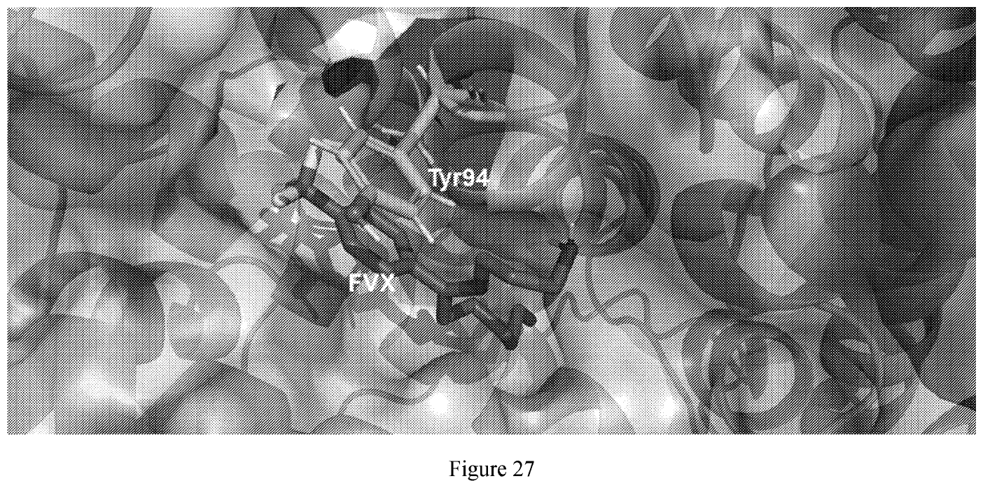

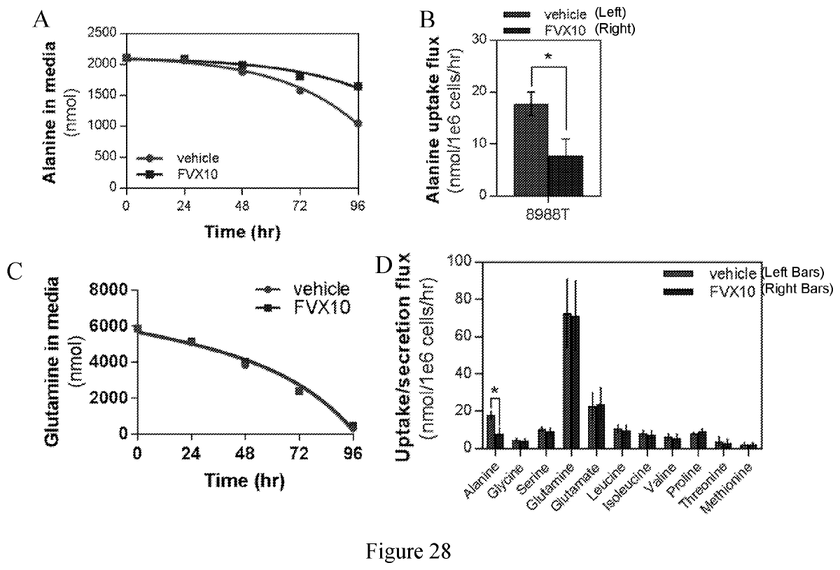

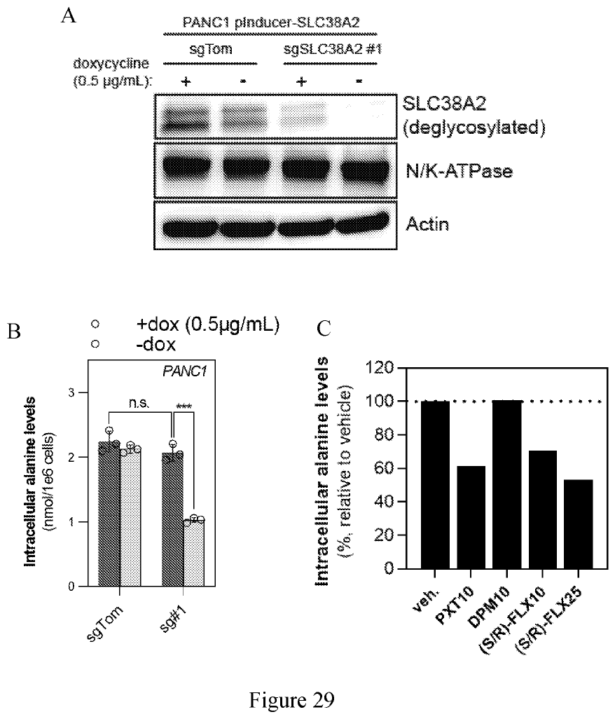

Results for these experiments are shown in FIGS. 13-29.

### Example 3

The following example provides a description of the methods of the present disclosure.

Provided is a method for a high-throughput fluorescent assay that may be used as a screening tool for targeting SLC38A2/alanine uptake. This tool may be desirable for SAR-type studies.

Generation of pLenti-blast-CMV-HyPerDAAO—To clone a lentiviral construct containing constitutively expressed HyPerDAAO under control of the CMV promoter, HyPerDAAO cDNA (Addgene, plasmid #119164) was amplified and isolated by PCR. The amplified fragment containing attB1 and attB2 sites at N- and C-terminal ends was recombined into pDONR221 (Invitrogen 12536017) using Gateway assembly (Invitrogen 11789100). HyPerDAAO was recombined into pLenti-blast-CMV (Addgene, plasmid #17451) using Gateway assembly (Invitrogen 11791100). Lentivirus were produced by transfecting 293T cells with pLenti-blast-CMV-HyPerDAAO, pMD2.G (Addgene, plasmid #12259), and psPAX2 (Addgene, plasmid #12260) using standard Lipofectamine 3000 (ThermoFisher) protocol. Cells were infected and selected with blasticidin (10 μg/mL) for ˜7-10 days.

Fluorescent detection of alanine uptake—MiaPaCa2 cells expressing HyPerDAAO were plated at roughly 75% confluency in black, clear bottom 96-well plates in custom DMEM (US Biological D9800) deficient in phenol red and supplemented with 10% dialyzed FBS (Gibco 26400044) a day prior to the assay. Wells were washing two times with two volumes of HBSS to remove serum immediately before adding 1-10 mM of D-Alanine (Sigma) and/or inhibitors: 2 mM of alpha-(Methylamino)isobutyric acid (MeAIB) or 1-20 μM of fluvoxamine (FVX), fluoxetine (FLX), blonanserin (BNS), paroxetine (PXT), or sertraline (SRT). Plates were immediately inserted into a SpectraMax M5 (Molecular Devices) heated at 37° C. Kinetic fluorescence was measured by sequential excitation at 420 nm and 480 nm and emission was measured at 530 nm every 0.1-1 minute for ˜60 minutes. After subtracting background fluorescence (wells containing no cells and HBSS), the ratio of oxidized HyPer (RFU₄₈₀ₙₘ) to non-oxidized HyPer (RFU₄₂₀ₙₘ) was calculated for each condition. Wells containing 10 mM L-Alanine (Sigma) which does not react with D-Amino acid oxidase (DAAO) was used as a negative control. Compounds that inhibited the increase in RFU480 nm/RFU420 nm were validated by treating cells with 1 mM L-alanine and vehicle or 1-20 μM compound for 1-3 hours and measuring intracellular metabolite levels by GC-MS.

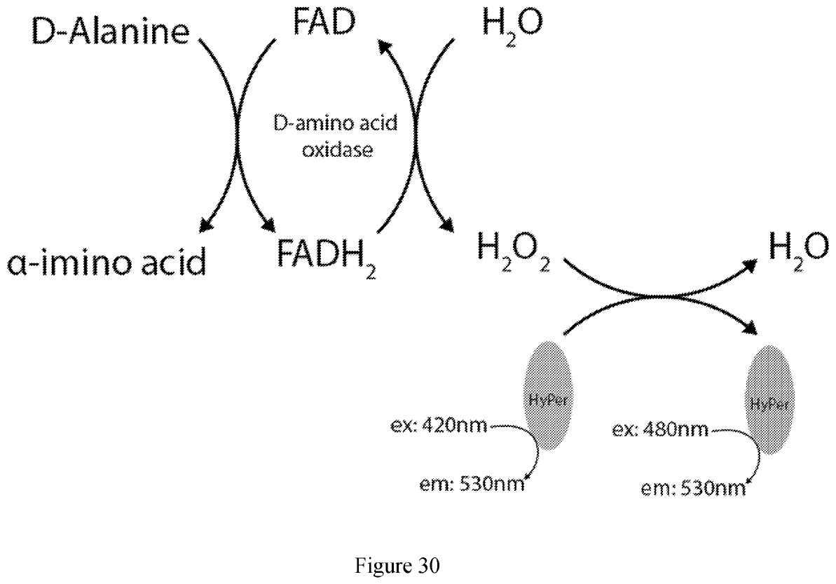

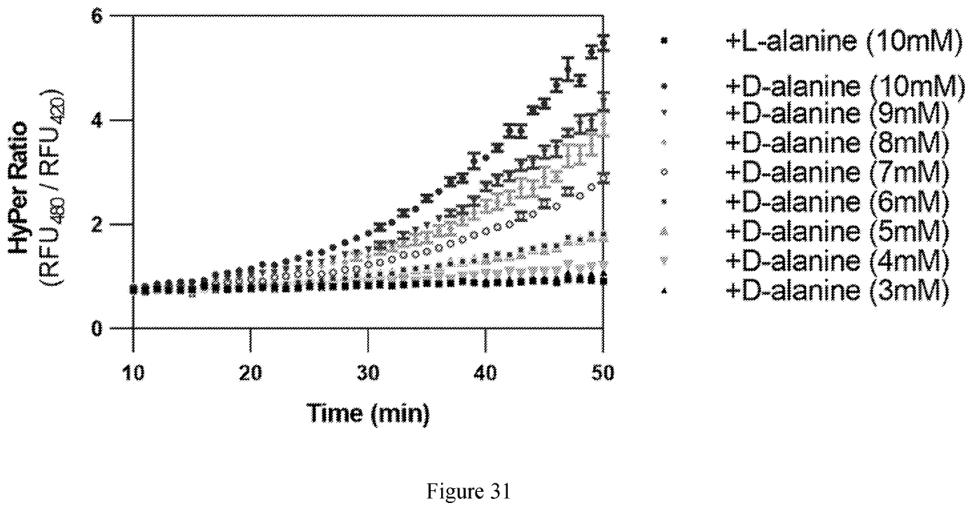

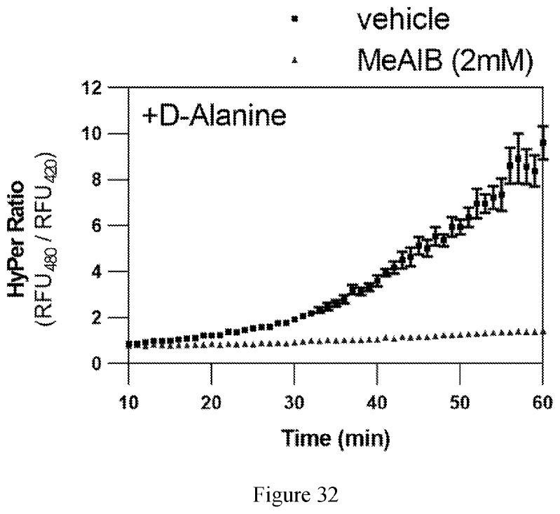

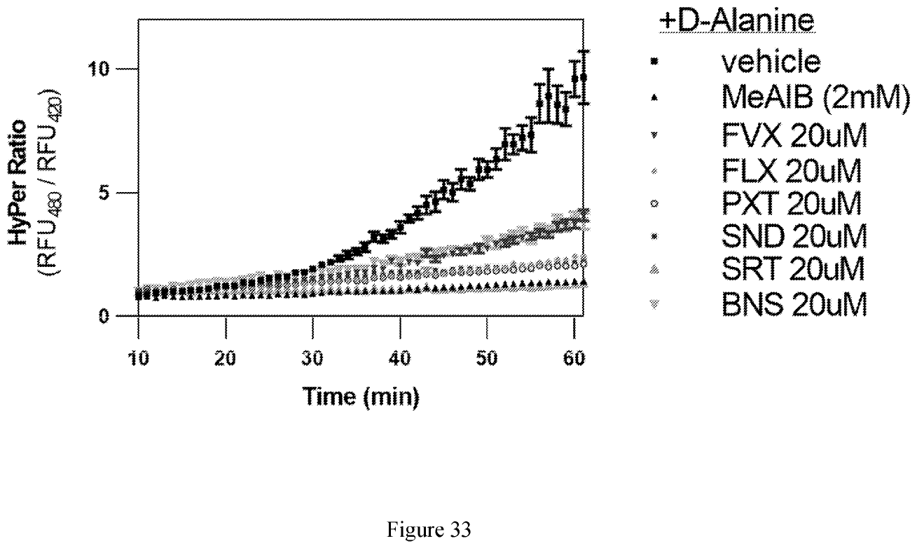

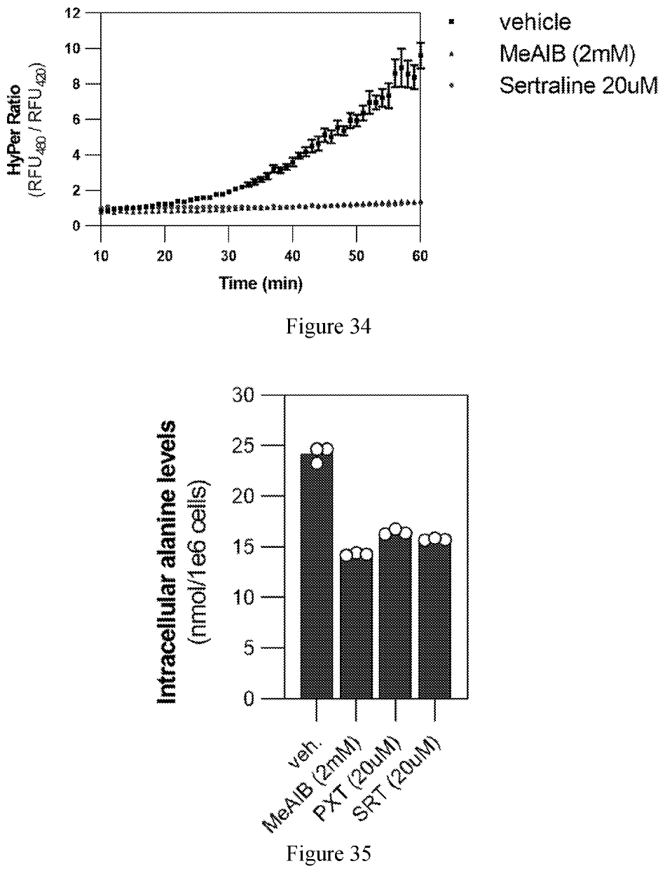

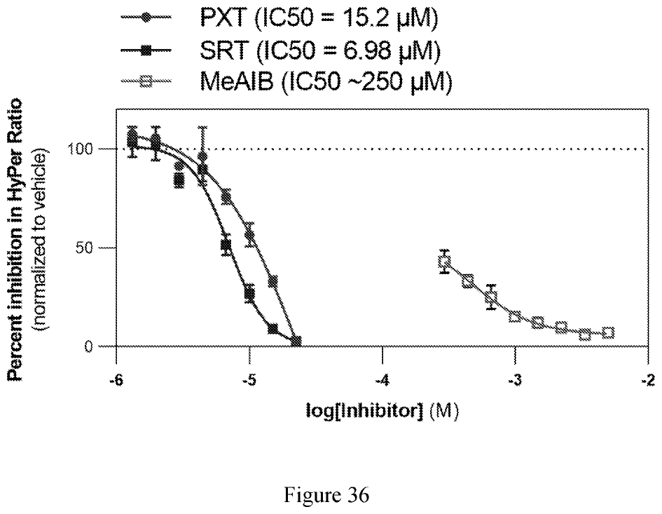

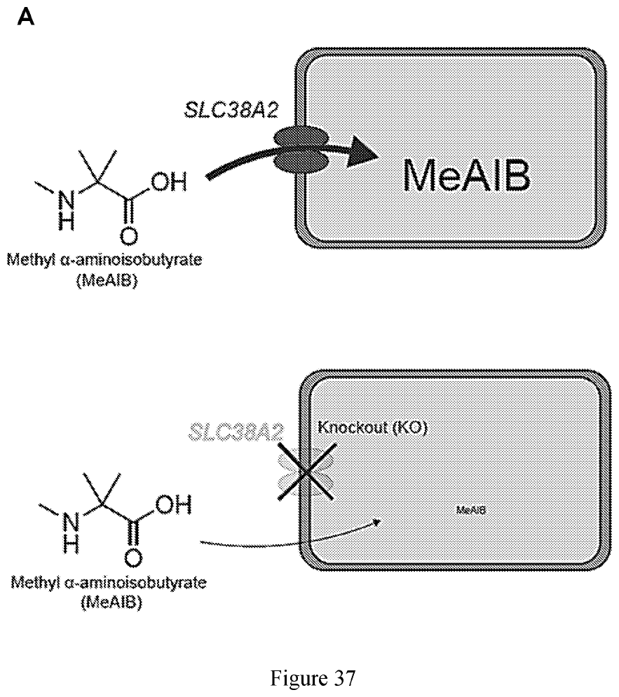

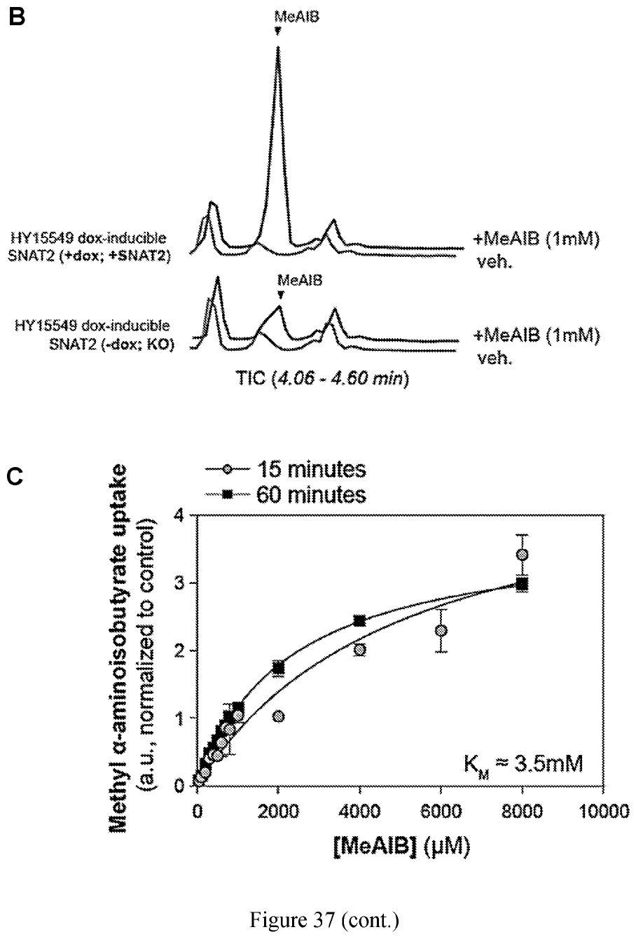

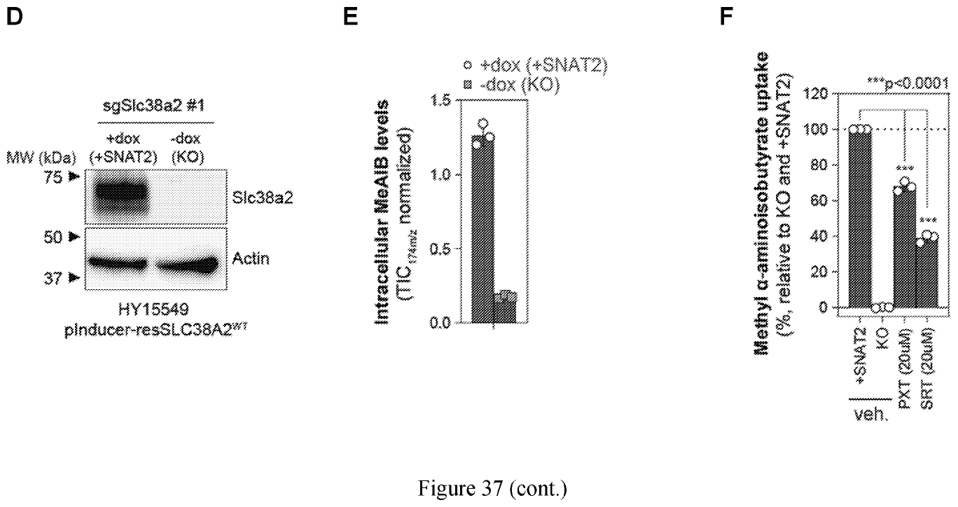

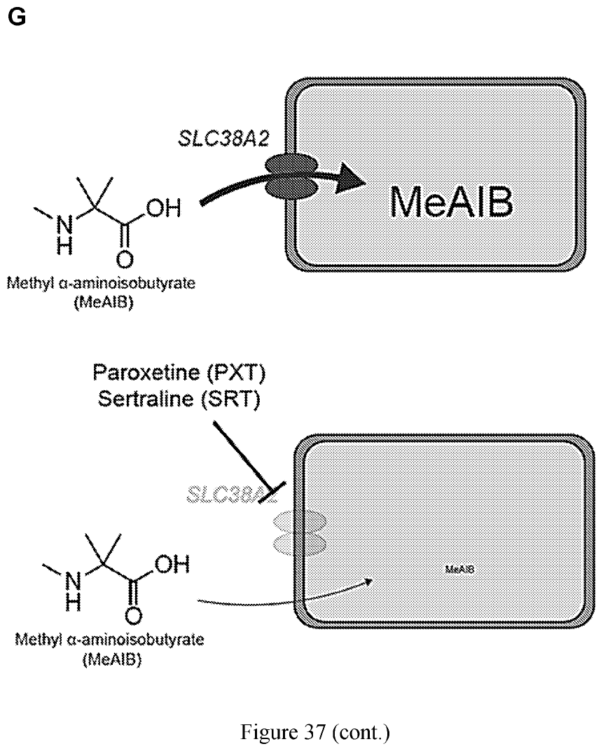

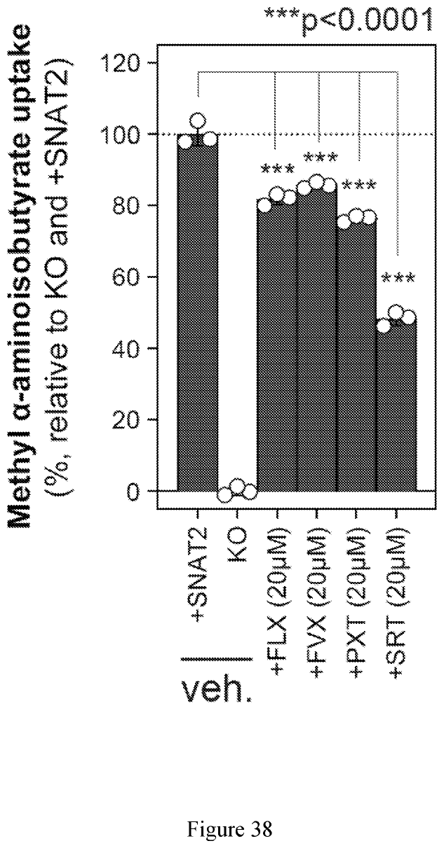

Results from these experiments is described in FIGS. 30-36.

Although the present disclosure has been described with respect to one or more particular examples, it will be understood that other examples of the present disclosure may be made without departing from the scope of the present disclosure.

1. A method of inhibiting SLC38A2 in a pancreatic cell and/or inhibiting alanine uptake by a pancreatic cell and/or inhibiting growth of pancreatic cells, comprising contacting the cell with an inhibitor of the expression or function of SLC38A2, wherein SLC38A2 is inhibited and/or alanine uptake by the pancreatic cell is inhibited and/or the growth of pancreatic cells is inhibited.

2. The method of claim 1, wherein the pancreatic cell(s) is/are pancreatic cancer cell(s).

3. The method of claim 2, wherein the pancreatic cancer cell(s) is/are pancreatic ductal adenocarcinoma cells.

4. The method of claim 1, wherein the SLC38A2 inhibitor is an antidepressant.

5. The method of claim 4, wherein the antidepressant is a serotonin reuptake inhibitor (SSRI), a tricyclic antidepressant (TCA), a tetracyclic antidepressant (TeCA), serotonin norepinephrine reuptake inhibitor (SNRI), a reversible inhibitor of monoamine oxidase-A (RIM-A), a 5-hydroxytryptamine receptor inhibitor (5-HTRi), or a combination thereof.

6. The method of claim 5, wherein the antidepressant is an SSRI chosen from fluvoxamine, fluoxetine, paroxetine, sertraline, and the like.

7. The method of claim 5, wherein the antidepressant is the TCA amitriptyline.

8. The method of claim 5, wherein the antidepressant is the TeCA ciclopramine.

9. The method of claim 4, wherein the dosage of the antidepressant is 1 nM to 100 mM.

10. The method of claim 1, wherein the SLC38A2 inhibitor is an antibody, antigen binding fragment thereof or a modification thereof, wherein the SLC38A2 inhibitor is directed to an epitope of SLC38A2 protein.

11. The method of claim 1, wherein the SLC38A2 inhibitor is an interfering RNA (RNAi) molecule or a dsRNA.

12. The method of claim 11, wherein the RNAi molecule is shRNA or siRNA.

13. The method of claim 1, wherein SLC38A2 is inhibited by disruption of a sequence encoding SLC38A2.

14. The method of claim 13, wherein the disruption is via CRISPR.

15. The method of claim 1, wherein the method is performed on an individual in need of treatment.

16. The method of claim 15, wherein the individual has been diagnosed with pancreatic cancer.

17. The method of claim 16, wherein the pancreatic cancer is pancreatic ductal adenocarcinoma.

18. The method of claim 17, wherein the method is used in combination with resection of the pancreatic tumor.

19. A method of identifying whether a pancreatic tumor is cancerous or non-cancerous, comprising:
obtaining a sample of pancreatic cells from the pancreatic tumor; and
identifying the location of the SLC38A2 protein in the cells;
wherein localization of the SLC38A2 protein in the plasma membrane is indicative of a cancerous tumor and intracellular localization of SLC38A2 in non-plasma membrane domains is indicative of a non-cancerous tumor.

20. The method of claim 19, wherein localization of SLC38A2 is identified by using an antibody, an antigen binding fragment thereof, or a modification thereof, wherein the antibody, the antigen binding fragment thereof, or the modification thereof is directed to an epitope of the SLC38A2 protein.

21. The method of claim 20, wherein the antibody of a fragment or modification thereof is detectably labeled.

22. The method of claim 19, further comprising treating an individual having the pancreatic tumor that is cancerous.

23. The method of claim 19, wherein the identifying step comprises imaging.

24. A method of identifying an inhibitor of SLC38A2, comprising:
determining cellular localization of SLC38A2 protein in a pancreatic cell in the presence and absence of a candidate inhibitor,
wherein a decrease in localization of SLC38A2 protein in the plasma membrane compartment in the presence of the candidate inhibitor compared to a control is indicative of a suitable inhibitor of SLC38A2.

25. The method of claim 24, wherein the determining step comprises imaging.

26. The method of claim 24, wherein the control does not comprise an inhibitor or the candidate inhibitor.

27. A method of identifying an inhibitor of SLC38A2, comprising:
determining alanine uptake in a pancreatic cell in the presence or absence of a candidate inhibitor,
wherein a decrease in alanine uptake in the presence of the candidate inhibitor compared to a control is indicative of a suitable inhibitor of SLC38A2.

28. The method of claim 27, wherein the control does not comprise an inhibitor or the candidate inhibitor.

29. A method for identifying a compound that inhibits alanine uptake in pancreatic cells, comprising:
performing a first measurement of alanine concentration in a cellular media comprising pancreatic cells;
contacting the pancreatic cells with the compound;
performing a second measurement of alanine concentration in the cellular media comprising pancreatic cells; and
determining if there is a change in the alanine concentration of the cellular media,
wherein an increase of alanine concentration in the cellular media correlates to an inhibition of alanine uptake and the compound is identified as inhibiting alanine uptake via the increase of alanine concentration in the cellular media.

30. The method of claim 29, wherein the pancreatic cell(s) is/are pancreatic cancer cell(s).

31. The method of claim 30, wherein the pancreatic cancer cell(s) is/are pancreatic ductal adenocarcinoma cell(s).

32. The method of claim 29, wherein a binding energy of a candidate inhibitor to SLC38A2 is determined via in silico molecular docking analysis.

33. The method of claim 29, wherein a binding energy of −1 to −10 kcal/mol indicates a suitable candidate inhibitor.

34. A method of inhibiting SLC1A4 in a pancreatic cell, comprising contacting the cell with an inhibitor of the expression or function of SLC1A4.


SEQUENCE LISTING (8 sequences)

SEQ ID NO: 1 - DNA, 22 bp, Artificial Sequence
  Primer
gcaagctgaccctgaagttcat

SEQ ID NO: 2 - DNA, 19 bp, Artificial Sequence
  Primer
ggagaagatactgtggcaa

SEQ ID NO: 3 - DNA, 21 bp, Artificial Sequence
  Primer
gaataccaagagttgtttcta

SEQ ID NO: 4 - DNA, 21 bp, Artificial Sequence
  Primer
tgtacaccagggatctgtttg

SEQ ID NO: 5 - DNA, 21 bp, Artificial Sequence
  Primer
cttcaccaatttgctcgtcat

SEQ ID NO: 6 - DNA, 19 bp, Artificial Sequence
  Primer
gccacgagttcgagatcga

SEQ ID NO: 7 - DNA, 20 bp, Artificial Sequence
  Primer
taatctgagcaatgcgattg

SEQ ID NO: 8 - DNA, 20 bp, Artificial Sequence
  Primer
tcttatgccatggctaatac